(19) United States
(12) Patent Application Publication
(10) Pub. No.: US 2008/0004274 A1
(43) Pub. Date: Jan. 3, 2008

(54) Novel biaromatic compounds that modulate PPAR type receptors and cosmetic/pharmaceutical compositions comprised thereof

(75) Inventors: Philippe Diaz, Houston, TX (US); Corinne Millois Barbuis, Saint-Raphael (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 11/812,048

(22) Filed: Jun. 14, 2007

Related U.S. Application Data
(63) Continuation of application No. PCT/EP05/14197, filed on Dec. 7, 2005.
(60) Provisional application No. 60/638,134, filed on Dec. 23, 2004.

(30) Foreign Application Priority Data
Dec. 14, 2004 (FR) 0413276

(51) Int. Cl. A61K 31/404; A61K 31/5377; A61P 17/00; C07D 209/40; C07D 413/12

(52) U.S. Cl. 514/235.2; 514/418; 544/144; 548/486

(57) ABSTRACT

Novel biaromatic compounds having the general formula (i) below: 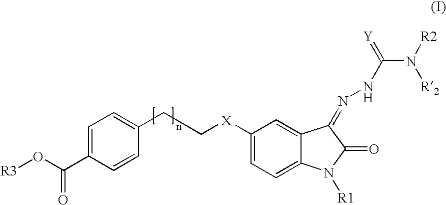 and cosmetic/pharmaceutical compositions comprised thereof are useful in human or veterinary medicine (in dermatology and also in the fields of cardiovascular diseases, of immune diseases and/or of diseases related to the metabolism of lipids) or, alternatively, in cosmetic compositions.

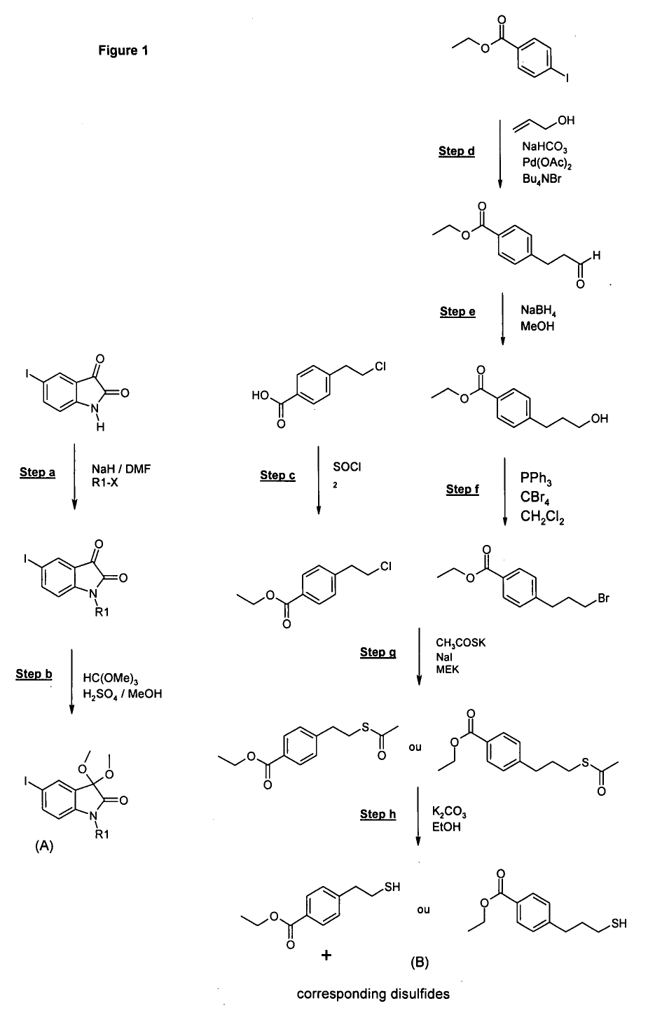

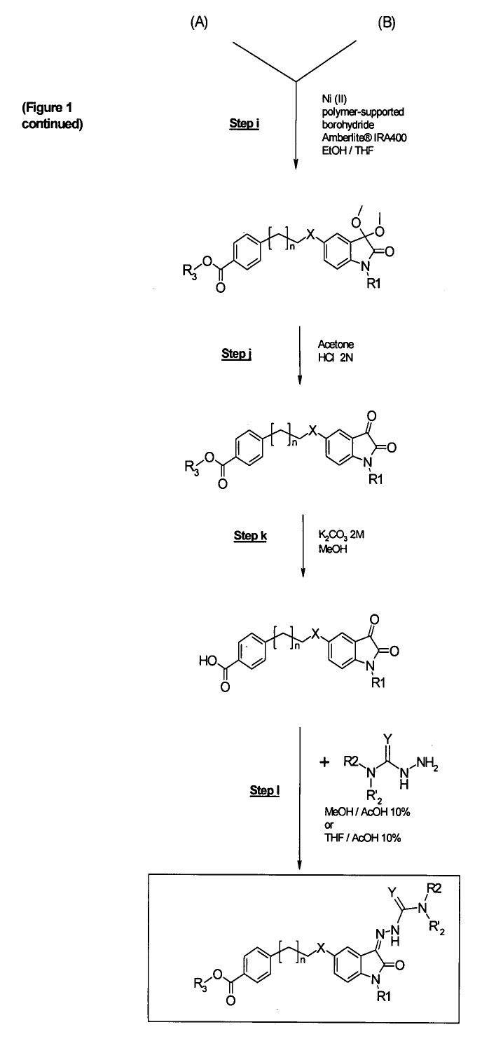

### CROSS-REFERENCE TO PRIORITY/PCT/PROVISIONAL APPLICATIONS

This application claims priority under 35 U.S.C. §119 of FR 04/13276, filed Dec. 14, 2004, and of Provisional Application No. 60/638,134, filed Dec. 23, 2004, and is a continuation of PCT/EP 2005/014197 filed Dec. 7, 2005 and designating the United States, published in the English language as WO 2006/063863 A2 on Jun. 22, 2006, each hereby expressly incorporated by reference in its entirety and each assigned to the assignee hereof.

### BACKGROUND OF THE INVENTION

1. Technical Field of the Invention

The present invention relates, as novel and useful industrial products, to a novel class of compounds that modulate the Peroxisome Proliferator-Activated Receptor type receptors (PPARs). This invention also relates to the process for preparing same and their formulation into pharmaceutical compositions for use in human or veterinary medicine, or, alternatively, into cosmetic compositions.

2. Description of Background and/or Related and/or Prior Art

The activity of receptors of PPAR type has been the subject of numerous studies. See, for example, the publication entitled “Differential Expression of Peroxisome Proliferator-Activated Receptor Subtypes During the Differentiation of Human Keratinocytes”, Michel Rivier et al., J. Invest. Dermatol., 111, (1998), 1116-1121, in which is listed a large number of bibliographic references relating to PPAR type receptors. See also the file entitled “The PPARs: From orphan receptors to Drug Discovery”, Timothy M. Willson, Peter J. Brown, Daniel D. Sternbach, and Brad R. Henke, J. Med. Chem., 43, (2000), 527-550.

The PPAR receptors activate transcription by binding to DNA sequence elements known as the peroxisome proliferator response elements (PPRE), in the form of a heterodimer with the retinoid X receptors (known as RXRs).

Three subtypes of human PPAR have been identified and described: PPARα, PPARγ and PPARδ (or NUC1). PPARα is mainly expressed in the liver, whereas PPARδ is ubiquitous.

It is described in WO 98/32444 that PPARα selective compounds play a role in the barrier function and the differentiation of the stratum corneum.

Of the three subtypes, PPARγ is the one that has been the most extensively studied. All the references suggest a critical role of PPARγ in the regulation of differentiation of adipocytes, where it is strongly expressed. It also plays a key role in systemic lipid homeostasis.

It has especially been described in WO 96/33724 that PPARγ-selective compounds, such as a prostaglandin-J2 or -D2, are potential active agents for the treatment of obesity and diabetes.

### SUMMARY OF THE INVENTION

The present invention features a novel class of compounds that modulate PPARs.

Thus, the present invention features novel biaromatic compounds having the general formula (I) below: 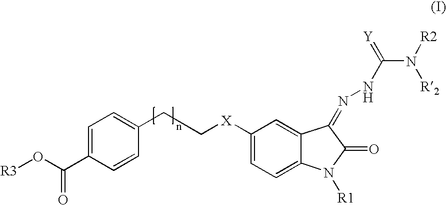
in which:

R₁ is an alkyl radical having from 1 to 10 carbon atoms and preferably from 1 to 7 carbon atoms, an aralkyl radical or an aryl radical;

R2 is a hydrogen atom, a linear or branched alkyl radical having from 1 to 7 carbon atoms, a substituted or unsubstituted aryl radical, a substituted or unsubstituted phenylsulfonyl radical, a substituted or unsubstituted heteroaryl radical, an aralkyl radical or a heterocyclic radical;

R′2 is a hydrogen atom;

with the proviso that R2 and R′2 can together form a heterocycle;

R3 is a hydrogen atom, an alkyl radical having from 1 to 3 carbon atoms, a polyether, an aryl radical, an aralkyl radical, a heteroaryl radical, a monohydroxyalkyl radical or a polyhydroxyalkyl radical;

X is S, CH₂, N or O;

Y is an oxygen or sulfur atom;

n is 1 or2;

and the possible geometrical isomers, which are pure or as a mixture, in all proportions, of the said compounds of formula (I), and also the salts thereof.

### BRIEF DESCRIPTION OF THE DRAWING

The FIGURE of Drawing shows a variety of reaction schemes for the ultimate preparation of the biaromatic compounds according to the invention.

### DETAILED DESCRIPTION OF BEST MODE AND SPECIFIC/PREFERRED EMBODIMENTS OF THE INVENTION

For the compounds of formula (I) presented above, the term “geometrical isomer” means syn/anti isomerism. More particularly, the double bond bonding the nitrogen to the ring, which is present in various compounds of general formula (I), may be of syn or anti configuration. These geometrical isomers, which may or may not be pure, alone or as a mixture, form an integral part of the compounds of formula (I).

When the compounds according to the invention are in the form of a salt, they are preferably a salt of an alkali metal or alkaline-earth metal, or, alternatively, a zinc salt or salts of an organic amine.

According to the present invention, the alkyl radicals having from 1 to 10 carbon atoms, or from 1 to 7 carbon atoms, are linear or branched radicals containing, respectively, from 1 to 10 or from 1 to 7 carbon atoms. Preferably, the alkyl radicals having from 1 to 7 carbon atoms are methyl, ethyl, n-propyl, n-butyl, tert-butyl, n-pentyl, n-hexyl or n-heptyl radicals.

The term “aralkyl radical” means a benzyl or phenethyl radical.

The term “aryl radical” means a phenyl radical, which may be mono- or disubstituted with one or more atoms or radicals selected from a halogen atom, a CF₃ radical and a methyl radical.

The term “substituted phenylsulfonyl radical” means a phenylsulfonyl radical substituted with a methyl group, preferably in the para position.

The term “heteroaryl” means an aryl radical interrupted with one or more hetero atoms, such as a thiophenyl, thiazolyl or imidazolyl radical, optionally substituted with at least one halogen, an alkyl radical having from 1 to 12 carbon atoms, an alkoxy radical having from 1 to 7 carbon atoms, an aryl radical, a nitro function, a polyether radical, a heteroaryl radical, a benzoyl radical, an alkyl ester group, a carboxylic acid, a hydroxyl group optionally protected with an acetyl or benzoyl group, or an amino function optionally protected with an acetyl or benzoyl group or optionally substituted with at least one alkyl having from 1 to 12 carbon atoms.

The term “heterocycle” preferably means a morpholino or imidazolidine-2,4-dione radical.

The term “polyether radical” means a polyether radical having from 1 to 6 carbon atoms interrupted with at least one oxygen atom, such as methoxymethoxy, ethoxymethoxy or methoxyethoxymethoxy radicals.

The term “monohydroxyalkyl radical” means a radical having from 1 to 6 carbon atoms and preferably having from 2 to 3 carbon atoms, especially a 2-hydroxyethyl, 2-hydroxypropyl or 3-hydroxypropyl radical.

The term “polyhydroxyalkyl radical” means a radical having from 3 to 6 carbon atoms and from 2 to 5 hydroxyl groups, such as 2,3-dihydroxypropyl, 2,3,4-trihydroxybutyl or 2,3,4,5-tetrahydroxypentyl radicals.

The term “halogen atom” preferably means a fluorine, chlorine or bromine atom.

Among the compounds of formula (I) above falling within the scope of the present invention, especially representative are the following compounds (alone or as a mixture):
1. 4-{2-[3-((anilinocarbonyl)hydrazono)-2-oxo-1-pentyl-2,3-dihydro-1H-indol-5-ylsulfanyl]ethyl}benzoic acid;
2. 4-{2-[3-((anilinocarbonyl)hydrazono)-2-oxo-1-pentyl-2,3-dihydro-1H-indol-5-ylsulfanyl]ethyl}benzoic acid;
3. 4-{2-[3-((anilinocarbonyl)hydrazono)-2-oxo-1-methyl-2,3-dihydro-1H-indol-5-ylsulfanyl]ethyl}benzoic acid;
4. 4-{2-[3-((anilinocarbonyl)hydrazono)-2-oxo-1-pentyl-2,3-dihydro-1H-indol-5-ylsulfanyl]ethyl}benzoic acid;
5. 4-[((3Z)-3-{[(benzylamino)carbonothioyl]hydrazono}-2-oxo-1-methyl-2,3-dihydro-1H-indol-5-ylsulfanyl]ethyl}benzoic acid;
6. 4-{2-[3-(benzylaminocarbonothioylhydrazono)-2-oxo-1-pentyl-2,3-dihydro-1H-indol-5-ylsulfanyl]ethyl}benzoic acid;
7. 4-{2-[3-(benzylaminocarbonothioylhydrazono)-2-oxo-1-benzyl-2,3-dihydro-1H-indol-5-ylsulfanyl]ethyl}benzoic acid;
8. 4-{2-[3-((anilinocarbonyl)hydrazono)-2-oxo-1-benzyl-2,3-dihydro-1H-indol-5-ylsulfanyl]ethyl}benzoic acid;
9. 4-{2-[3-(anilinocarbonothioylhydrazono)-2-oxo-1-pentyl-2,3-dihydro-1H-indol-5-ylsulfanyl]ethyl}benzoic acid;
10. 4-{2-[3-(ethylaminocarbonothioylhydrazono)-2-oxo-1-pentyl-2,3-dihydro-1H-indol-5-ylsulfanyl]ethyl}benzoic acid;
11. methyl 4-{2-[3-((4-fluoroanilino)carbonylhydrazono)-2-oxo-1-phenethyl-2,3-dihydro-1H-indol-5-ylsulfanyl]ethyl}benzoate;
12. methyl 4-{2-[3-((anilinocarbonyl)hydrazono)-2-oxo-1-butyl-2,3-dihydro-1H-indol-5-ylsulfanyl]ethyl}benzoate;
13. methyl 4-{2-[3-((anilinocarbonyl)hydrazono)-2-oxo-1-heptyl-2,3-dihydro-1H-indol-5-ylsulfanyl]ethyl}benzoate;
14. 4-{2-[3-((anilinocarbonyl)hydrazono)-2-oxo-1-butyl-2,3-dihydro-1H-indol-5-ylsulfanyl]ethyl}benzoic acid;
15. 4-{2-[3-((anilinocarbonyl)hydrazono)-2-oxo-1-heptyl-2,3-dihydro-1H-indol-5-ylsulfanyl]ethyl}benzoic acid;
16. 4-{2-[3-((anilinocarbonyl)hydrazono)-2-oxo-1-phenethyl-2,3-dihydro-1H-indol-5-ylsulfanyl]ethyl}benzoic acid;
17. methyl 4-{2-[3-((3-fluoroanilinocarbonyl)hydrazono)-2-oxo-1-phenethyl-2,3-dihydro-1H-indol-5-ylsulfanyl]ethyl}benzoate;
18. 4-{2-[3-((3-fluoroanilinocarbonyl)hydrazono)-2-oxo-1-butyl-2,3-dihydro-1H-indol-5-yl-sulfanyl]ethyl}benzoic acid;
19. methyl 4-{2-[3-((3-trifluoromethylanilinocarbonyl)hydrazono)-2-oxo-1-phenethyl-2,3-dihydro-1H-indol-5-ylsulfanyl]ethyl}benzoate;
20. 4-{2-[3-((3-trifluoromethylanilinocarbonyl)hydrazono)-2-oxo-1-heptyl-2,3-dihydro-1H-indol-5-ylsulfanyl]ethyl}benzoic acid;
21. methyl 4-{2-[3((2-chloroanilinocarbonyl)hydrazono)-2-oxo-1-phenethyl-2,3-dihydro-1H-indol-5-ylsulfanyl]ethyl}benzoate;
22. 4-{2-[3((2-chloroanilinocarbonyl)hydrazono)-2-oxo-1-heptyl-2,3-dihydro-1H-indol-5-yl-sulfanyl]ethyl}benzoic acid;
23. 4-{2-[3((2-chloroanilinocarbonyl)hydrazono)-2-oxo-1-phenethyl-2,3-dihydro-1H-indol-5-ylsulfanyl]ethyl}benzoic acid;
24. 4-{2-[3((4-fluoroanilinocarbonyl)hydrazono)-2-oxo-1-heptyl-2,3-dihydro-1H-indol-5-yl-sulfanyl]ethyl}benzoic acid;
25. 4-{2-[3((4-fluoroanilinocarbonyl)hydrazono)-2-oxo-1-phenethyl-2,3-dihydro-1H-indol-5-ylsulfanyl]ethyl}benzoic acid;
26. 4-{2-[3((4-methylbenzenesulfonamido)carbonylhydrazono)-2-oxo-1-phenethyl-2,3-dihydro-1H-indol-5-ylsulfanyl]ethyl}benzoic acid;
27. methyl 4-{2-[3((3,4-dichloroanilinocarbonyl)hydrazono)-2-oxo-1-1-phenethyl-2,3-dihydro-1H-indol-5-ylsulfanyl]ethyl}benzoate;
28. methyl 4-(2-{1-butyl-3-[(morpholine-4-carbonyl)hydrazono]-2-oxo-2,3-dihydro-1H-indol-5-ylsulfanyl}ethyl)benzoate;
29. methyl 4-(2-{1-heptyl-3-[(morpholine-4-carbonyl)hydrazono]-2-oxo-2,3-dihydro-1H-indol-5-ylsulfanyl}ethyl)benzoate;
30. methyl 4-(2-{3-[(morpholine-4-carbonyl)hydrazono]-2-oxo-1-phenethyl-2,3-dihydro-1H-indol-5-ylsulfanyl}ethyl)benzoate;
31. 4-(2-{1-butyl-3-[(morpholine4-carbonyl)hydrazono]-2-oxo-2,3-dihydro-1H-indol-5-yl-sulfanyl}ethyl)benzoic acid;
32. 4-(2-{1-heptyl-3-[(morpholine-4-carbonyl)hydrazono]-2-oxo-2,3-dihydro-1H-indol-5-ylsulfanyl}ethyl)benzoic acid;
33. 4-(2-{3-[(morpholine-4-carbonyl)hydrazono]-2-oxo-1-phenethyl-2,3-dihydro-1H-indol-5-ylsulfanyl}ethyl)benzoic acid;
34. methyl 4-{2-[3-((2-chloro-4-trifluoromethylanilinocarbonyl)hydrazono)-2-oxo-1-phen-ethyl-2,3-dihydro-1H-indol-5-ylsulfanyl]ethyl}benzoate;
35. 4-{2-[3((2-chloro-4-trifluoromethylanilinocarbonyl)hydrazono)-2-oxo-1-phenethyl-2,3-dihydro-1H-indol-5-ylsulfanyl]ethyl}benzoic acid;
36. methyl 4-{2-[3((tert-butylamino)carbonylhydrazono)-2-oxo-1-phenethyl-2,3-dihydro-1H-indol-5-ylsulfanyl]ethyl}benzoate;
37. ethyl 4-{2-[3(((anilinocarbonyl)hydrazono)-2-oxo-1-pentyl-2,3-dihydro-1H-indol-5-ylsulfanyl]ethyl}benzoate;
38. 4-{2-[3-((3-fluoroanilinocarbonyl)hydrazono)-2-oxo-1-heptyl-2,3-dihydro-1H-indol-5-ylsulfanyl]ethyl}benzoic acid;
39. 4-{2-[3((aminocarbonyl)hydrazono)-2-oxo-1-pentyl-2,3-dihydro-1H-indol-5-ylsulfanyl]ethyl}benzoic acid;
40. 4-[3-(3-((3-trifluoromethylanilinocarbonyl)hydrazono)-2-oxo-1-propyl-2,3-dihydro-1H-indol-5-ylsulfanyl)propyl]benzoic acid;
41. 4-[3-(3-((3-chloroanilinocarbonyl)hydrazono)-2-oxo-1-hexyl-2,3-dihydro-1H-indol-5-ylsulfanyl)propyl]benzoic acid;
42. 4-[3-(3-((2-chloroanilinocarbonyl)hydrazono)-2-oxo-1-propyl-2,3-dihydro-1H-indol-5-ylsulfanyl)propyl]benzoic acid;
43. 4-[3-(3-((4-fluoroanilinocarbonyl)hydrazono)-2-oxo-1-propyl-2,3-dihydro-1H-indol-5-ylsulfanyl)propyl]benzoic acid;
44. 4-{2-[3-((3-trifluoromethylanilinocarbonyl)hydrazono)-2-oxo-1-propyl-2,3-dihydro-1H-indol-5-ylsulfanyl]ethyl}benzoic acid;
45. 4-{2-[3-((2-chloro-3-trifluoromethylanilinocarbonyl)hydrazono)-2-oxo-1-propyl-2,3-dihydro-1H-indol-5-ylsulfanyl]ethyl}benzoic acid;
46. 4-[3-(3-((3-chloroanilinocarbonyl)hydrazono)-2-oxo-1-pentyl-2,3-dihydro-1H-indol-5-ylsulfanyl)propyl]benzoic acid; 47. 4-{2-[3((aminocarbonyl)hydrazono)-2-oxo-1-propyl-2,3-dihydro-1H-indol-5-ylsulfanyl]ethyl}benzoic acid;
48. 4-{3-[3((aminocarbonyl)hydrazono)-2-oxo-1-propyl-2,3-dihydro-1H-indol-5-ylsulfanyl]propyl}benzoic acid;
49. 4-[3-(3-((2-chloro-4-trifluoroanilinocarbonyl)hydrazono)-2-oxo-1-propyl-2,3-dihydro-1H-indol-5-ylsulfanyl)propyl]benzoic acid;
50. 4-{3-[3((aminocarbonyl)hydrazono)-2-oxo-1-hexyl-2,3-dihydro-1H-indol-5-ylsulfanyl]propyl}benzoic acid;
51. 4-{2-[3((thiophen-2-ylamine)carbonylhydrazono)-2-oxo-1-pentyl-2,3-dihydro-1H-indol-5-ylsulfanyl]ethyl}benzoic acid;
52. 4-{2-[3-((2-chloroanilinocarbonyl)hydrazono)-2-oxo-1-propyl-2,3-dihydro-1H-indol-5-ylsulfanyl]ethyl}benzoic acid;
53. 4-[3-(3-((3-chloroanilinocarbonyl)hydrazono)-2-oxo-1-propyl-2,3-dihydro-1H-indol-5-ylsulfanyl)propyl]benzoic acid;
54. 4-{2-[3-((3-chlororanilinocarbonyl)hydrazono)-2-oxo-1-propyl-2,3-dihydro-1H-indol-5-ylsulfanyl]ethyl}benzoic acid;
55. 4-{2-[3-((3,4-dichloroanilinocarbonyl)hydrazono)-2-oxo-1-pentyl-2,3-dihydro-1H-indol-5-ylsulfanyl]ethyl}benzoic acid;
56. 4-{2-[3-((3,4-dichloroanilinocarbonyl)hydrazono)-2-oxo-1-hexyl-2,3-dihydro-1H-indol-5-ylsulfanyl]ethyl}benzoic acid;
57. 4-{3-[3((aminocarbonyl)hydrazono)-2-oxo-1-pentyl-2,3-dihydro-1H-indol-5-ylsulfanyl]propyl}benzoic acid;
58. 4-(2-{3-[(morpholine-4-carbonyl)hydrazono]-2-oxo-1-propyl-2,3-dihydro-1H-indol-5-ylsulfanyl}ethyl)benzoic acid;
59. 4-[3-(3-((2-chloro-4-trifluoroanilinocarbonyl)hydrazono)-2-oxo-1-hexyl-2,3-dihydro-1H-indol-5-ylsulfanyl)propyl]benzoic acid;
60. 4-[3-(3-((3-fluoroanilinocarbonyl)hydrazono)-2-oxo-1-pentyl-2,3-dihydro-1H-indol-5-ylsulfanyl)propyl]benzoic acid;
61. 4-[3-(3-((3-fluoroanilinocarbonyl)hydrazono)-2-oxo-1-hexyl-2,3-dihydro-1H-indol-5-ylsulfanyl)propyl]benzoic acid;
62. 4-[3-(3-((anilinocarbonyl)hydrazono)-2-oxo-1-hexyl-2,3-dihydro-1H-indol-5-ylsulfanyl)propyl]benzoic acid;
63. 4-[3-(3-((3-fluoroanilinocarbonyl)hydrazono)-2-oxo-1-hexyl-2,3-dihydro-1H-indol-5-ylsulfanyl)ethyl]benzoic acid;
64. 4-(3-{1-hexyl-3-[(morpholine4-carbonyl)hydrazono]-2-oxo-2,3-dihydro-1H-indol-5-ylsulfanyl}propyl)benzoic acid;
65. 4-[3-(3-((anilinocarbonyl)hydrazono)-2-oxo-1-pentyl-2,3-dihydro-1H-indol-5-ylsulfanyl)propyl]benzoic acid;
66. 4-(3-{3-[(morpholine-4-carbonyl)hydrazono]-2-oxo-1-propyl-2,3-dihydro-1H-indol-5-ylsulfanyl}propyl)benzoic acid;
67. 4-(3-{3-[(morpholine4-carbonyl)hydrazono]-2-oxo-1-pentyl-2,3-dihydro-1H-indol-5-ylsulfanyl}propyl)benzoic acid;
68. 4-{2-[3-((4-fluoroanilinocarbonyl)hydrazono)-2-oxo-1-propyl-2,3-dihydro-1H-indol-5-ylsulfanyl]ethyl}benzoic acid;
69. 4-[3-(3-((3-fluoroanilinocarbonyl)hydrazono)-2-oxo-1-propyl-2,3-dihydro-1H-indol-5-ylsulfanyl)propyl]benzoic acid;
70. 4-{2-[3-((anilinocarbonyl)hydrazono)-2-oxo-1-hexyl-2,3-dihydro-1H-indol-5-ylsulfanyl]ethyl}benzoic acid;
71. 4-[3-(3-((4-fluoroanilinocarbonyl)hydrazono)-2-oxo-1-pentyl-2,3-dihydro-1H-indol-5-ylsulfanyl)propyl]benzoic acid;
72. 4-(2-{3-[(morpholine-4-carbonyl)hydrazono]-2-oxo-1-pentyl-2,3-dihydro-1H-indol-5-ylsulfanyl}ethyl)benzoic acid;
73. 4-(2-{1-hexyl-3-[(morpholine-4-carbonyl)hydrazono]-2-oxo-2,3-dihydro-1H-indol-5-ylsulfanyl}ethyl)benzoic acid;
74. 4-[3-(3-((4-fluoroanilinocarbonyl)hydrazono)-2-oxo-1-hexyl-2,3-dihydro-1H-indol-5-ylsulfanyl)propyl]benzoic acid;
75. 4-[3-(3-((3-trifluoromethylanilinocarbonyl)hydrazono)-2-oxo-1-pentyl-2,3-dihydro-1H-indol-5-ylsulfanyl)propyl]benzoic acid;
76. 4-[3-(3-((3-trifluoromethylanilinocarbonyl)hydrazono)-2-oxo-1-hexyl-2,3-dihydro-1H-indol-5-ylsulfanyl)propyl]benzoic acid;
77. 4-{2-[3-((3-fluoroanilinocarbonyl)hydrazono)-2-oxo-1-pentyl-2,3-dihydro-1H-indol-5-ylsulfanyl]ethyl}benzoic acid;
78. 4-{2-[3-((3,4-dichloroanilinocarbonyl)hydrazono)-2-oxo-1-propyl-2,3-dihydro-1H-indol-5-ylsulfanyl]ethyl}benzoic acid;
79. 4-{2-[3-((anilinocarbonyl)hydrazono)-2-oxo-1-propyl-2,3-dihydro-1H-indol-5-ylsulfanyl]ethyl}benzoic acid;
80. 4-{2-[3((1,3-thiazol-2-amine)carbonylhydrazono)-2-oxo-1-pentyl-2,3-dihydro-1H-indol-5-ylsulfanyl]ethyl}benzoic acid;
81. 4-{2-[3-((3-trifluoromethylanilino)carbonylhydrazono)-2-oxo-1-hexyl-2,3-dihydro-1H-indol-5-ylsulfanyl]ethyl}benzoic acid;
82. 4-{2-[3-((4-fluoroanilino)carbonylhydrazono)-2-oxo-1-hexyl-2,3-dihydro-1H-indol-5-ylsulfanyl]ethyl}benzoic acid;
83. 4-{2-[3-((2-chloroanilino)carbonylhydrazono)-2-oxo-1-heptyl-2,3-dihydro-1H-indol-5-ylsulfanyl]ethyl}benzoic acid;
84. ethyl 4-{2-[3-((2-chloroanilinocarbonyl)hydrazono)-2-oxo-1-heptyl-2,3-dihydro-1H-indol-5-ylsulfanyl]ethyl}benzoate;
85. 4-{2-[3((1H-imidazol-2-amine)carbonylhydrazono)-2-oxo-1-pentyl-2,3-dihydro-1H-indol-5-ylsulfanyl]ethyl}benzoic acid;
86. 4-{2-[3((thiophen-2-ylamine)carbonylhydrazono)-2-oxo-1-heptyl-2,3-dihydro-1H-indol-5-ylsulfanyl]ethyl}benzoic acid;
87. 4-{2-[3((1,3-thiazol-2-amine)carbonylhydrazono)-2-oxo-1-heptyl-2,3-dihydro-1H-indol-5-ylsulfanyl]ethyl}benzoic acid;
88. 4-{2-[3((1H-imidazol-2-amine)carbonylhydrazono)-2-oxo-1-heptyl-2,3-dihydro-1H-indol-5-ylsulfanyl]ethyl}benzoic acid;
89. 4-{3-[3((thiophen-2-ylamine)carbonylhydrazono)-2-oxo-1-pentyl-2,3-dihydro-1H-indol-5-ylsulfanyl]propyl}benzoic acid;
90. 4-{3-[3((1,3-thiazol-2-amine)carbonylhydrazono)-2-oxo-1-pentyl-2,3-dihydro-1H-indol-5-ylsulfanyl]propyl}benzoic acid;
91. 4-{3-[3((1H-imidazol-2-amine)carbonylhydrazono)-2-oxo-1-pentyl-2,3-dihydro-1H-indol-5-ylsulfanyl]propyl}benzoic acid;
92. 4-{3-[3((thiophen-2-ylamine)carbonylhydrazono)-2-oxo-1-heptyl-2,3-dihydro-1H-indol-5-ylsulfanyl]propyl}benzoic acid;
93. 4-{3-[3((1,3-thiazol-2-amine)carbonylhydrazono)-2-oxo-1-heptyl-2,3-dihydro-1H-indol-5-ylsulfanyl]propyl}benzoic acid;
94. 4-{3-[3((1H-imidazol-2-amine)carbonylhydrazono)-2-oxo-1-heptyl-2,3-dihydro-1H-indol-5-ylsulfanyl]propyl}benzoic acid.

According to the present invention, the compounds of formula (I) that are more particularly preferred are those that satisfy at least one of the following conditions:

R1 is selected from an alkyl radical selected from methyl, propyl, butyl, pentyl, hexyl and heptyl radicals, a benzyl radical or a phenethyl radical;

R2 is selected from a hydrogen atom, an alkyl radical selected from ethyl and tert-butyl radicals, an unsubstituted phenyl radical, a phenyl radical mono- or disubstituted with a fluorine or chlorine atom or a CF₃ radical, a benzyl radical, a phenylsulfonyl radical substituted with a methyl radical, or an unsubstituted heteroaryl radical selected from thiophenyl, thiazolyl and imidazolyl radicals;

R′2 is a hydrogen atom;

with the proviso that R2 and R′2 can together form a morpholino radical,

R3 is selected from a hydrogen atom and a methyl or ethyl radical.

A general description of the preparation of the compounds of general formula (I) of the FIGURE of Drawing attached hereto is given below.

The reaction scheme described in the FIGURE of Drawing is a general scheme for obtaining the compounds according to the invention.

The compounds of general formula (I) may be obtained by coupling a thiol of formula (B) with the modified “5-iodoisatin” of formula (A). These starting compounds (A) and (B) are obtained in the following manner:

the amine function of the “5-iodoisatin” is alkylated (step a) and its ketone function is then protected (step b) to give the modified “5-iodoisatin” of formula (A);

the compounds of formula (B) are obtained via two different routes, depending on whether the final compounds of general formula (I) have a value n equal to 1 or 2. Thus, a) when n=1 the acid function of 4-chloroethylbenzoic acid is protected via esterification (step c);

b) when n=2: ethyl 4-iodobenzoate undergoes a substitution with an aldehyde (step d) and then a reduction (step e), followed by a halogenation (step f).

The compounds thus obtained in steps c and f undergo a thioacetylation (step g), followed by a partial hydrolysis of the thiol ester (step h) to finally obtain the ethyl thioalkylbenzoates of formula (B).

The coupling of an ethyl thioalkylbenzoate of formula (B) with the “5-iodoisatin” (A) is performed by using a metal catalyst, for instance nickel or palladium derivatives in the presence of a hydride donor, for instance sodium borohydride optionally in supported form (step i). The following step is a deprotection of the ketone (step j). The derivatives obtained are then optionally saponified via the action of a base, for instance K₂CO₃ as a 2 M solution in water in the presence of ethanol, to give the corresponding acids (step k).

The ester or acid obtained may then be subjected to the action of a semicarbazide or a thiosemicarbazide in a solvent such as ethanol in the presence of 10% acetic acid, to give the corresponding carbonylhydrazono or carbonylthioylhydrazono of general formula (I) (step I).

The compounds according to the invention have modulatory properties on PPAR type receptors. This activity on the PPARα, δ and γ receptors is measured in a transactivation test and quantified by means of the dissociation constant Kdapp (apparent), as described in Example 86.

The preferred compounds of the present invention have a dissociation constant Kdapp of less than or equal to 1000 nM and advantageously less than or equal to 500 nM for at least one of the PPAR subtypes.

The present invention also features the administration of the compounds of formula (I) as described above, as medicaments.

The present invention also features formulation of the compounds of formula (I) into compositions for regulating and/or restoring skin lipid metabolism.

The compounds according to the invention are particularly useful in the following fields of treatment:

1) for treating dermatological complaints, conditions or afflictions associated with a keratinization disorder relating to cell differentiation and proliferation, especially for treating common acne, comedones, polymorphs, acne rosacea, nodulocystic acne, acne conglobata, senile acne, and secondary acnes such as solar acne, medication-related acne or occupational acne;

2) for treating other types of keratinization disorders, especially ichthyosis, ichthyosiform conditions, Darier's disease, palmoplantar keratoderma, leukoplakia and leukoplakiform conditions, and cutaneous or mucous (buccal) lichen;

3) for treating other dermatological complaints, conditins or afflictions having an inflammatory immunoallergic component, with or without cell proliferation disorder, and especially all forms of psoriasis, whether cutaneous, mucous or ungual, and even psoriatic rheumatism, or cutaneous atopy, such as eczema, or respiratory atopy, or, alternatively, gingival hypertrophy;

4) for treating all dermal or epidermal proliferations, whether benign or malignant, and whether of viral origin or otherwise, such as common warts, flat warts and verruciform epidermodysplasia, oral or florid papillomatoses, T lymphoma, and proliferations that may be induced by ultraviolet radiation, especially in the case of basal cell and spinal cell epithelioma, and also any precancerous skin lesion such as keratoacanthomas;

5) for treating other dermatological disorders, conditions or afflictions such as immune dermatoses, such as lupus erythematosus, immune bullous diseases and collagen diseases, such as scleroderma;

6) in the treatment of dermatological or general complaints or conditions having an immunological component;

7) in the treatment of skin disorders caused by exposure to UV radiation, and also for repairing or combating aging of the skin, whether photoinduced or chronological aging, or for reducing actinic pigmentations and keratosis, or any pathology associated with chronological or actinic aging, such as xerosis;

8) for combating sebaceous function disorders, such as the hyperseborrhoea of acne or simple seborrhoea or seborrhoeic dermatitis;

9) for preventing or treating cicatrization disorders, or for preventing or repairing stretchmarks;

10) in the treatment of pigmentation disorders, such as hyperpigmentation, melasma, hypopigmentation or vitiligo;

11) in the treatment of lipid metabolism complaints or conditions, such as obesity, hyperlipidaemia, non-insulin-dependent diabetes or syndrome X;

12) in the treatment of inflammatory complaints or conditions such as arthritis;

13) in the treatment or prevention of cancerous or precancerous conditions;

14) in the prevention or treatment of alopecia of various origins, especially alopecia caused by chemotherapy or radiation;

15) in the treatment of disorders of the immune system, such as asthma, type I sugar diabetes, multiple sclerosis or other selective dysfunctions of the immune system; or

16) in the treatment of complaints or conditions of the cardiovascular system, such as arteriosclerosis or hypertension.

The present invention also features pharmaceutical or cosmetic compositions, preferably dermatological compositions, comprising, formulated into a physiologically acceptable medium, at least one compound of formula (I) as defined above. The term “physiologically acceptable medium” means a medium that is compatible with the skin, the integuments, mucous membranes and tissues.

The compositions according to the invention may be administered enterally, parenterally, topically or ocularly. The pharmaceutical composition is preferably packaged in a form that is suitable for topical application.

Via the enteral route, the composition, more particularly the pharmaceutical composition, may be in the form of tablets, gel capsules, dragees, syrups, suspensions, solutions, powders, granules, emulsions, lipid or polymeric microspheres, nanospheres or vesicles allowing a controlled release. Via the parenteral route, the composition may be in the form of solutions or suspensions for infusion or for injection.

The compounds according to the invention are generally administered at a daily dose of about 0.001 mg/kg to 100 mg/kg of body weight, in 1 to 3 dosage intakes.

The compounds are administered systemically, at a concentration generally of from 0.001% to 10% by weight and preferably from 0.01% to 1% by weight, relative to the weight of the composition.

Via the topical route, the pharmaceutical composition according to the invention is more particularly useful for treating the skin and mucous membranes and may be in the form of ointments, creams, milks, pomades, powders, impregnated pads, syndets, solutions, gels, sprays, mousses, suspensions, lotions, sticks, shampoos or washing bases. It may also be in the form of suspensions of lipid or polymeric microspheres, nanospheres or vesicles or polymer patches and hydrogels allowing a controlled release. This topical composition may be in anhydrous form, in aqueous form or in the form of an emulsion.

The compounds are administered topically at a concentration generally of from 0.001% to 10% by weight and preferably from 0.01% to 1% by weight, relative to the total weight of the composition.

The compounds of formula (I) according to the invention also find an application in cosmetics, in particular in body and hair hygiene and more particularly for regulating and/or restoring skin lipid metabolism.

A subject of the invention is thus also the cosmetic use of a composition comprising, in a physiologically acceptable support, at least one of the compounds of formula (I) for body or hair hygiene.

The cosmetic compositions according to the invention containing, in a cosmetically acceptable support, at least one compound of formula (I) or an optical or geometrical isomer thereof or a salt thereof, may especially be in the form of a cream, a milk, a lotion, a gel, suspensions of lipid or polymeric microspheres, nanospheres or vesicles, impregnated pads, solutions, sprays, mousses, sticks, shampoos or washing bases.

The concentration of compound of formula (I) in the cosmetic composition is from 0.001% to 3% by weight relative to the total weight of the composition.

The present invention also features a cosmetic regime or regimen for enhancing the skin, which entails applying to the skin a composition comprising at least one compound of formula (I).

The pharmaceutical and cosmetic compositions as described above may also contain inert or even pharmacodynamically active additives as regards the pharmaceutical compositions, or combinations of these additives, and especially:

wetting agents;

flavor enhancers;

preservatives such as para-hydroxybenzoic acid esters;

stabilizers;

humidity regulators;

pH regulators;

osmotic pressure modifiers;

emulsifiers;

UV-A and UV-B screening agents;

antioxidants, such as α-tocopherol, butylhydroxyanisole or butylhydroxytoluene, superoxide dismutase, ubiquinol or certain metal-chelating agents;

depigmenting agents such as hydroquinone, azelaic acid, caffeic acid or kojic acid;

emollients;

moisturizers, for instance glycerol, PEG 400, thiamorpholinone and derivatives thereof, or urea;

anti-seborrhoeic or anti-acne agents, such as S-carboxymethylcysteine, S-benzylcysteamine, salts thereof or derivatives thereof, or benzoyl peroxide;

antibiotics, for instance erythromycin and its esters, neomycin, clindamycin and its esters, and tetracyclines;

anti-fungal agents such as ketoconazole or polymethylene-4,5-isothiazolidones-3;

agents for promoting regrowth of the hair, for instance Minoxidil (2,4-diamino-6-piperidinopyrimidine 3-oxide) and its derivatives, Diazoxide (7-chloro-3-methyl-1,2,4-benzothiadiazine 1,1-dioxide) and Phenytoin (5,4-diphenylimidazolidine-2,4-dione);

non-steroidal anti-inflammatory agents;

carotenoids, and especially β-carotene;

anti-psoriatic agents such as anthralin and its derivatives;

eicosa-5,8,11,14-tetraynoic acid and eicosa-5,8,11-triynoic acid, and esters and amides thereof;

retinoids, i.e., RAR or RXR receptor ligands, which may be natural or synthetic;

corticosteroids or oestrogens;

α-hydroxy acids and α-keto acids or derivatives thereof, such as lactic acid, malic acid, citric acid, glycolic acid, mandelic acid, tartaric acid, glyceric acid or ascorbic acid, and also the salts, amides or esters thereof, or β-hydroxy acids or derivatives thereof, such as salicylic acid and the salts, amides or esters thereof;

ion-channel blockers such as potassium-channel blockers;

or, alternatively, more particularly for the pharmaceutical compositions, in combination with medicaments known to interfere with the immune system (for example cyclosporin, FK 506, glucocorticoids, monoclonal antibodies, cytokines or growth factors, etc.).

Of course, one skilled in the art will take care to select the optional compound(s) to be added to these compositions such that the advantageous properties intrinsically associated with the present invention are not, or are not substantially, adversely affected by the envisaged addition.

In order to further illustrate the present invention and the advantages thereof, the following examples of specific active compounds are given, as are the results of the biological activities of such compounds and specific formulations thereof, it being understood that same are intended only as illustrative and in nowise limitative. In said examples to follow, all parts and percentages are given by weight, unless otherwise indicated.

### EXAMPLE 1

### Synthesis of 4-{2-[3Z-((anilinocarbonyl)hydrazono)-2-oxo-1-pentyl-2,3-dihydro-1H-indol-5-ylsulfanyl]ethyl}benzoic acid (syn)

### a) Preparation of 5-iodo-1-pentyl-1H-indole-2,3-dione

4.86 g (0.121 mol) of 60% sodium hydride are added portionwise to a mixture of 30.03 g (0.11 mol) of 5-iodoisatin in 300 ml of dimethylformamide. The reaction medium is stirred at room temperature for 2 hours. 18.3 g (1.121 mol) of 1-bromopentane dissolved in 20 ml of dimethylformamide are then added dropwise. The reaction medium is stirred at room temperature overnight. The reaction medium is then poured into saturated ammonium chloride solution and extracted with ethyl acetate. The organic phases are combined, washed with water, dried over magnesium sulfate and concentrated on a rotary evaporator under vacuum. The solid is taken up in a heptane/ethyl acetate mixture, filtered and dried. 34.57 g (92%) of the expected compound are collected, in the form of a vermilion-red powder.

### b) Preparation of 5-iodo-3,3-dimethoxy-1-pentyl-1,3-dihydro-indol-2-one

32.10 ml (0.59 mol) of concentrated sulfuric acid are added dropwise to a mixture of 33.57 g (0.098 mol) of 5-Iodo-1-pentyl-1H-indole-2,3-dione in 960 ml of methanol/trimethoxymethane (1:1). The reaction medium is then stirred at room temperature overnight. The reaction medium is poured into ice-water solution and neutralized to pH 8 with sodium hydrogen carbonate. The desired product is extracted with ethyl ether. The organic phases are combined, washed with water, dried over magnesium sulfate and concentrated on a rotary evaporator under vacuum. The paste obtained (39.16 g) is purified by chromatography on a column of silica (dichloromethane). After evaporating off the solvent, the expected compound (36.75 g; 96%), is isolated in the form of an orange paste.

### c) Preparation of ethyl 4-(2-chloroethyl)benzoate

5.8 ml (0.08 mol) of thionyl chloride are added dropwise to a mixture of 11.28 g (0.061 mol) of 4-(2-chloroethyl)benzoic acid in 36 ml of ethanol heated to 50° C. The reaction medium is then refluxed for 6 hours. The reaction medium is poured into ice-water solution and neutralized with sodium hydrogen carbonate. The desired product is extracted with ethyl ether. The organic phases are combined, washed with water, dried over magnesium sulfate and concentrated on a rotary evaporator under vacuum. 12.70 g (98%) of the expected compound are collected, in the form of a yellow oil.

### g) Preparation of ethyl 4-(2-acetylsulfanylethyl)benzoate

A mixture of 12.24 g (0.057 mol) of ethyl 4-(2-chloroethyl)benzoate, 13.15 g (0.115 mol) of potassium thioacetate and 78 mg (0.6% by mass) of sodium iodide in 250 ml of methyl ethyl ketone is heated at reflux for 5 hours. On cooling to room temperature, the reaction medium is then poured into saturated solution ammonium chloride and extracted with ethyl ether. The organic phases are combined, washed with water, dried over magnesium sulfate and concentrated on a rotary evaporator under vacuum. 14.45 g (100%) of the expected compound are obtained in the form of a brown oil.

### h) Preparation of ethyl 4-(2-mercaptoethyl)benzoate

A mixture of 14.7 g (0.058 mol) of ethyl 4-(2-acetylsulfanylethyl)benzoate and 10.42 g (0.075 mol) of potassium carbonate in 220 ml of ethanol is stirred at room temperature overnight. The reaction medium is poured into ice-cold 2N hydrochloric acid solution and extracted with ethyl ether. The organic phases are combined, washed with water, dried over magnesium sulfate and concentrated on a rotary evaporator under vacuum. The brown oil obtained (12.2 g) is purified by chromatography on a column of silica (90/10 heptane/ethyl acetate). After evaporating off the solvents, the expected compound (3.87 g; 32%) is isolated in the form of a brown liquid along with 7.40 g (61%) of corresponding disulfide, isolated in the form of a brown oil.

### i) Preparation of ethyl 4-[2-(3,3-dimethoxy-2-oxo-1-pentyl-2,3-dihydro-1H-indol-5-ylsulfanyl)ethyl]benzoate

A solution of 2.21 g (10 mmol) of ethyl 4-(2-mercaptoethyl)benzoate obtained in Example 1 h in 10 ml of tetrahydrofuran is added to a mixture of 8.40 g (21 mmol) of polymer-supported borohydride resin Amberlite® IRA400 (2.5 mmol/g) (Aldrich: 32864-2), 90 mg of bis(bipyridine)nickel (II) bromide (Organometallics 1985, 4, 657-661) and 2.73 g (7 mmol) of 5-iodo-3,3-dimethoxy-1-pentyl-1,3-dihydroindol-2-one in 70 ml of ethanol. The mixture is heated at 70° C. for 5 hours. The reaction medium is filtered and the filtrate is concentrated on a rotary evaporator under vacuum. The product obtained (3.83 g) is purified by chromatography on a column of silica (95/5 dichloromethane/ethyl acetate). After evaporating off the solvents, the expected compound (2.73 g; 83%) is isolated in the form of a green oil.

### j) Preparation of ethyl 4-[2-(2,3-dioxo-1-pentyl-2,3-dihydro-1H-indol-5-ylsulfanyl)ethyl]benzoate

39 ml (78 mmol) of 2N hydrochloric acid are added dropwise to a mixture of 2.51 g (5.3 mmol) of ethyl 4-[2-(3,3-dimethoxy-2-oxo-1-pentyl-2,3-dihydro-1H-indol-5-ylsulfanyl)ethyl]benzoate in 82 ml of acetone heated to 40° C. The reaction medium is stirred at 40° C. for 24 hours. The reaction medium is poured into a mixture of water and ethyl acetate, and the phases are then separated by settling. The desired product is extracted with ethyl acetate and the organic phases are combined, washed with water, dried over magnesium sulfate and concentrated on a rotary evaporator under vacuum. The product obtained (2.74 g) is purified by chromatography on a column of silica (80/20 heptane/ethyl acetate). After evaporating off the solvents, the expected compound (1.60 g; 71%) is isolated in the form of a flaky blood-red solid.

### k) Synthesis of 4-[2-(2,3-dioxo-1-pentyl-2,3-dihydro-1H-indol-5-ylsulfanyl)ethyl]benzoic acid

A mixture of 1.28 g (3.0 mmol) of ethyl 4-[2-(2,3-dioxo-1-pentyl-2,3-dihydro-1H-indol-5-ylsulfanyl)ethyl]benzoate in 50 ml of methanol and 23 ml (46 mmol) of a 2 M solution of potassium carbonate in water is heated at 60-65° C. for 3 hours and then concentrated on a rotary evaporator under vacuum. Water is added to the residue obtained. The solution is acidified by addition of concentrated hydrochloric acid. The desired product is extracted with ethyl acetate and the organic phases are combined, washed with water, dried over magnesium sulfate and concentrated on a rotary evaporator under vacuum. The product is purified by chromatography on a column of silica (30/70 heptane/ethyl acetate). After evaporating off the solvents, the expected compound (875 mg; 73%) is isolated in the form of a dark orange powder.

### l) Synthesis of 4-{2-[3Z-((anilinocarbonyl)hydrazono)-2-oxo-1-pentyl-2,3-dihydro-1H-indol-5-ylsulfanyl]ethyl}benzoic acid

A solution of 125 mg (0.75 mmol) of 4-phenylsemicarbazide in 10 ml of 10% acetic acid in ethanol is added to a mixture of 300 mg (0.75 mmol) of 4-[2-(2,3-dioxo-1-pentyl-2,3-dihydro-1H-indol-5-ylsulfanyl)ethyl]benzoic acid in 10 ml of 10% acetic acid in tetrahydrofuran. The reaction medium is stirred gently in the absence of light overnight. The reaction mixture is filtered (see Example 2). The filtrate obtained is evaporated to give the expected product (97 mg), which is purified by preparative HPLC (acetonitrile/water). After evaporating off the solvents, the expected compound (40 mg; 10%) is isolated in the form of an orange powder. (m.p.=160-162° C.)

¹H NMR (CDCl₃, 400 MHz): 0.90 (t, 3H), 1.35 (m, 4H), 1.69 (m, 2H), 2.98 (t, 2H), 3.18 (t, 2H), 3.75 (t, 2H), 6.86 (d, Ar, 1H), 7.12 (t, Ar, 1H), 7.24 (d, Ar, 2H), 7.37 (t, Ar, 2H), 7.43 (d, Ar, 1H), 7.61 (d, Ar, 2H), 7.70 (s, Ar, 1H), 7.97 (d, Ar, 2H), 8.44 (s, 1H), 12.12 (s, 1H).

### EXAMPLE 2

### Synthesis of 4-{2-[3E-((anilinocarbonyl)hydrazono)-2-oxo-1-pentyl-2,3-dihydro-1H-indol-5-ylsulfanyl]ethyl}benzoic acid (anti)

Steps a), b), c), g), h), i), j) and k) are the same as those of Example 1.

### l) Synthesis of 4-{2-[3E-((anilinocarbonyl)hydrazono)-2-oxo-1-pentyl-2,3-dihydro-1H-indol-5-ylsulfanyl]ethyl}benzoic acid

A solution of 125 mg (0.75 mmol) of 4-phenylsemicarbazide in 10 ml of 10% acetic acid in ethanol is added to a mixture of 300 mg (0.75 mmol) of 4-[2-(2,3-dioxo-1-pentyl-2,3-dihydro-1H-indol-5-ylsulfanyl)ethyl]benzoic acid in 10 ml of 10% acetic acid in tetrahydrofuran. The reaction medium is stirred gently overnight in the absence of light. The precipitate obtained is filtered and dried. 338 mg (85%) of product are obtained in the form of a pale orange solid. (m.p.=164-166° C.)

¹H NMR (CDCl₃, 400 MHz): 0.90 (t, 3H), 1.35 (m, 4H), 1.69 (m, 2H), 2.98 (t, 2H), 3.22 (t, 2H), 3.76 (t, 2H), 6.86 (d, 1H), 7.10 (t, Ar, 1H), 7.25 (d, Ar, 2H), 7.33 (t, Ar, 2H), 7.46 (d, Ar, 1H), 7.61 (d, Ar, 2H), 7.95 (d, Ar, 2H), 8.22 (s, Ar, 1H), 8.88 (s, 1H), 10.37 (s, 1H).

### EXAMPLE 3

### Synthesis of 4-{2-[3-((anilinocarbonyl)hydrazono)-2-oxo-1-methyl-2,3-dihydro-1H-indol-5-ylsulfanyl]ethyl}benzoic acid

### a) Preparation of 5-iodo-1-methyl-1H-indole-2,3-dione

1.77 g (0.044 mol) of 60% sodium hydride are added portionwise to a mixture of 11.0 g (0.04 mol) of 5-iodoisatin in 110 ml of dimethylformamide. The reaction medium is stirred at room temperature for 2 hours. 2.75 ml (0.044 mol) of iodomethane are then added dropwise. The reaction medium is stirred at room temperature overnight. The reaction medium is then poured into ice-water solution, neutralized to pH 8 with sodium hydrogen carbonate and extracted with ethyl ether. The organic phases are combined, washed with water, dried over magnesium sulfate and concentrated on a rotary evaporator under vacuum. The solid is taken up in a heptane/dichloromethane mixture, filtered and dried. 10.50 g (91%) of the expected compound are collected in the form of a blood-red powder. (m.p.=160-162° C.)

### b) Preparation of 5-iodo-3,3-dimethoxy-1-methyl-1,3-dihydroindol-2-one

In a manner similar to that of Example 1(b), by reacting 8.35 ml (0.156 mol) of concentrated sulfuric acid and 7.48 g (0.026 mol) of 5-iodo-1-methyl-1H-indole-2,3-dione in 260 ml of methanol/trimethoxymethane (1:1), 6.80 g (85%) of the expected derivative are obtained in the form of an apricot-colored powder. (m.p.=101-103° C.).

Steps c), g) and h) are the same as those of Example 1.

### i) Preparation of ethyl 4-[2-(3,3-dimethoxy-2-oxo-1-methyl-2,3-dihydro-1H-indol-5-ylsulfanyl)ethyl]benzoate

A solution of 2.83 g (6.75 mmol) of ethyl 4-(2-mercaptoethyl)benzoate obtained in Example 1h in disulfide form in 10 ml of tetrahydrofuran is added to a mixture of 10.80 g (27 mmol) of polymer-supported borohydride resin Amberlite® IRA400 (2.5 mmol/g) (Aldrich: 32864-2), 112 mg of bis(bipyridine)nickel (II) bromide (Organometallics 1985, 4, 657-661) and 2.80 g (9 mmol) of 5-iodo-3,3-dimethoxy-1-methyl-1,3-dihydroindol-2-one in 90 ml of ethanol. The mixture is heated at 70° C. overnight. The reaction medium is filtered and the filtrate is concentrated on a rotary evaporator under vacuum. The product obtained (3.47 g) is purified by chromatography on a column of silica (80/20 heptane/ethyl acetate). After evaporating off the solvents, the expected compound (1.98 g; 53%) is isolated in the form of a pale green oil.

### j) Preparation of ethyl 4-[2-(2,3-dioxo-1-methyl-2,3-dihydro-1H-indol-5-ylsulfanyl)ethyl]benzoate

34.5 ml (69 mmol) of 2N hydrochloric acid are added dropwise to a mixture of 1.96 g (4.7 mmol) of ethyl 4-[2-(3,3-dimethoxy-2-oxo-1-methyl-2,3-dihydro-1H-indol-5-ylsulfanyl)ethyl]benzoate in 60 ml of acetone heated to 40° C. The reaction medium is stirred at 40° C. for 14 hours. The reaction medium is poured into a mixture of water and ethyl acetate, and the phases are then separated by settling. The desired product is extracted with ethyl acetate and the organic phases are combined, washed with water, dried over magnesium sulfate and concentrated on a rotary evaporator under vacuum. The product obtained (1.75 g) is purified by chromatography on a column of silica (65/35 heptane/ethyl acetate). After evaporating off the solvents, the expected compound (1.27 g; 73%) is isolated in the form of a Bordeaux-red powder.

### k) Preparation of 4-[2-(2,3-dioxo-1-methyl-2,3-dihydro-1H-indol-5-ylsulfanyl)ethyl]benzoic acid

A mixture of 1.27 g (3.44 mmol) of ethyl 4-[2-(2,3-dioxo-1-methyl-2,3-dihydro-1H-indol-5-ylsulfanyl)ethyl]benzoate in 50 ml of methanol and 26 ml (52 mmol) of a 2M solution of potassium carbonate in water is heated at 55° C. for 2 hours 30 minutes and then concentrated on a rotary evaporator under vacuum. Water is added to the residue obtained. The solution is acidified by addition of concentrated hydrochloric acid. The desired product is extracted with ethyl acetate and the organic phases are combined, washed with water, dried over magnesium sulfate and concentrated on a rotary evaporator under vacuum. The solid is taken up in ethyl ether, filtered and dried. 1.086 g (92%) of the expected compound are collected in the form of an orange powder.

### l) Synthesis of 4-{2-[3-((anilinocarbonyl)hydrazono)-2-oxo-1-methyl-2,3-dihydro-1H-indol-5-ylsulfanyl]ethyl}benzoic acid

This step was performed by parallel chemistry in a 96-well plate.

0.5 ml (0.040 mmol) of a solution of 102 mg of 4-phenylsemicarbazide in 8.5 ml of 10% acetic acid in ethanol is added to 0.5 ml (0.036 mmol) of a mixture of 86 mg of 4-[2-(2,3-dioxo-1-methyl-2,3-dihydro-1H-indol-5-ylsulfanyl)ethyl]benzoic acid in 7 ml of 10% acetic acid in tetrahydrofuran. The reaction medium is stirred at room temperature overnight. The precipitate is filtered off (S) and, after evaporating off the filtrate, the expected compound (8 mg) is isolated in the form of an orange solid.

### EXAMPLE 4

### Synthesis of 4-{2-[3-((anilinocarbonyl)hydrazono)-2-oxo-1-pentyl-2,3-dihydro-1H-indol-5-ylsulfanyl]ethyl}benzoic acid

Steps a), b), c), g) and h) are the same as those of Example 1.

### i) Preparation of ethyl 4-[2-(3,3-dimethoxy-2-oxo-1-pentyl-2,3-dihydro-1H-indol-5-ylsulfanyl)ethyl]benzoate

In a manner similar to that of Example 1(i), by reacting 2.84 g (13.5 mmol) of ethyl 4-(2-mercaptoethyl)benzoate obtained in Example 1h in 10 ml of tetrahydrofuran, 10.8 g (27 mmol) of polymer-supported borohydride resin Amberlite® IRA400 (2.5 mmol/g) (Aldrich: 32864-2), 112 mg of bis(bipyridine)nickel (II) bromide (Organometallics 1985, 4, 657-661) and 3.50 g (9 mmol) of 5-iodo-3,3-dimethoxy-1-pentyl-1,3-dihydroindol-2-one in 90 ml of ethanol, 3.78 g (89%) of the expected derivative are obtained in the form of an ochre-colored oil.

### j) Preparation of ethyl 4-[2-(2,3-dioxo-1-pentyl-2,3-dihydro-1H-indol-5-ylsulfanyl)ethyl]benzoate

In a manner similar to that of Example 1(j), by reacting 55 ml (110 mmol) of 2N hydrochloric acid with a mixture of 3.51 g (7.44 mmol) of ethyl 4-[2-(3,3-dimethoxy-2-oxo-1-pentyl-2,3-dihydro-1H-indol-5-ylsulfanyl)ethyl]benzoate in 115 ml of acetone, 2.58 g (81%) of the expected derivative are obtained in the form of a flaky blood-red solid.

### k) Preparation of 4-[2-(2,3-dioxo-1-pentyl-2,3-dihydro-1H-indol-5-ylsulfanyl)ethyl]benzoic acid

In a manner similar to that of Example 1(k), by reacting 2.59 g (6.09 mmol) of ethyl 4-[2-(2,3-dioxo-1-pentyl-2,3-dihydro-1H-indol-5-ylsulfanyl)ethyl]benzoate in 105 ml of methanol and 46 ml (92 mmol) of a 2M solution of potassium carbonate in water, 1.00 9 (41%) of the expected derivative are obtained in the form of a brown powder.

### l) Synthesis of 4-{2-[3-((anilinocarbonyl)hydrazono)-2-oxo-1-pentyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 102 mg of 4-phenylsemicarbazide in 8.5 ml of 10% acetic acid in ethanol and 0.5 ml (0.036 mmol) of a mixture of 100 mg of 4-[2-(2,3-dioxo-1-pentyl-2,3-dihydro-1H-indol-5-ylsulfanyl)ethyl]benzoic acid in 3.5 ml of 10% acetic acid in tetrahydrofuran, 13 mg of the expected derivative are obtained in the form of an orange solid.

### EXAMPLE 5

### Synthesis of 4-[((3Z)-3-{[(benzylamino)carbonothioyl]hydrazono}-2-oxo-1-methyl-2,3-dihydro-1H-indol-5-ylsulfanyl]ethyl}benzoic acid

Steps a), b), c), g), h), i), j) and k) are the same as those of Example 3.

### l) Synthesis of 4-[((3Z)-3-{[(benzylamino)carbonothioyl]hydrazono}-2-oxo-1-methyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 122 mg of 4-benzylthiosemicarbazide in 8.5 ml of 10% acetic acid in ethanol and 0.5 ml (0.036 mmol) of a mixture of 86 mg of 4-[2-(2,3-dioxo-1-methyl-2,3-dihydro-1H-indol-5-ylsulfanyl)ethyl]benzoic acid in 7 ml of 10% acetic acid in tetrahydrofuran, 17 mg of the expected derivative are obtained in the form of an orange solid.

### EXAMPLE 6

### Synthesis of 4-{2-[3-(benzylaminocarbonothioylhydrazono)-2-oxo-1-pentyl-2,3-dihydro-1H-indol-5-ylsulfanyl]ethyl}benzoic acid

Steps a), b), c), g), h), i), j) and k) are the same as those of Example 4.

### l) Synthesis of 4-{2-[3-(benzylaminocarbonothioylhydrazono)-2-oxo-1-pentyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 122 mg of 4-benzylthiosemicarbazide in 8.5 ml of 10% acetic acid in ethanol and 0.5 ml (0.036 mmol) of a mixture of 100 mg of 4-[2-(2,3-dioxo-1-pentyl-2,3-dihydro-1H-indol-5-ylsulfanyl)ethyl]benzoic acid in 3.5 ml of 10% acetic acid in tetrahydrofuran, and after crystallization from ethyl ether, 17 mg of the expected derivative are obtained in the form of an orange solid.

### EXAMPLE 7

### Synthesis of 4-{2-[3-(benzylaminocarbonothioylhydrazono)-2-oxo-1-benzyl-2,3-dihydro-1H-indol-5-ylsulfanyl]ethyl}benzoic acid

### a) Preparation of 5-iodo-1-benzyl-1H-indole-2,3-dione

In a manner similar to that of Example 1(a), by reacting 4.86 g (0.121 mol) of 60% sodium hydride, 30.03 g (0.11 mol) of 5-iodoisatin in 300 ml of dimethylformamide and 20.7 g (1.121 mol) of benzyl bromide dissolved in 20 ml of dimethylformamide, 34.60 g (89%) of the expected derivative are obtained in the form of a vermilion-red powder.

### b) Preparation of 5-iodo-3,3-dimethoxy-1-benzyl-1,3-dihydroindol-2-one

In a manner similar to that of Example 1(b), by reacting 31.10 ml (0.57 mol) of concentrated sulfuric acid and 34.58 g (0.095 mol) of 5-iodo-1-benzyl-1H-indole-2,3-dione in 940 ml of methanol/trimethoxymethane (1:1), 38.48 g (99%) of the expected derivative are obtained in the form of an orange paste that subsequently crystallizes.

Steps c), g), h) are the same as those of Example 1.

### i) Preparation of ethyl 4-[2-(3,3-dimethoxy-2-oxo-1-benzyl-2,3-dihydro-1H-indol-5-ylsulfanyl)ethyl]benzoate

In a manner similar to that of Example 1(i), by reacting 2.83 g (6.75 mmol) of ethyl 4-(2-mercaptoethyl)benzoate obtained in Example 1h in disulfide form in 10 ml of tetrahydrofuran, 10.8 g (27 mmol) of polymer-supported borohydride resin Amberlite® IRA400 (2.5 mmol/g) (Aldrich: 32864-2), 112 mg of bis(bipyridine)nickel (II) bromide (Organometallics 1985, 4, 657-661) and 3.68 g (9 mmol) of 5-iodo-3,3-dimethoxy-1-benzyl-1,3-dihydroindol-2-one in 90 ml of ethanol, 2.92 g (66%) of the expected derivative are obtained in the form of a yellow oil.

### j) Preparation of ethyl 4-[2-(2,3-dioxo-1-benzyl-2,3-dihydro-1H-indol-5-ylsulfanyl)ethyl]benzoate

In a manner similar to that of Example 10), by reacting 56 ml (112 mmol) of 2N hydrochloric acid and 2.52 g (5.13 mmol) of ethyl 4-[2-(3,3-dimethoxy-2-oxo-1-benzyl-2,3-dihydro-1H-indol-5-ylsulfanyl)ethyl]benzoate in 80 ml of acetone, 1.31 g (57%) of the expected derivative are obtained in the form of purple beads.

### k) Preparation of 4-[2-(2,3-dioxo-1-benzyl-2,3-dihydro-1H-indol-5-ylsulfanyl)ethyl]benzoic acid

In a manner similar to that of Example 1(k), by reacting 1.32 g (2.96 mmol) of ethyl 4-[2-(2,3-dioxo-1-benzyl-2,3-dihydro-1H-indol-5-ylsulfanyl)ethyl]benzoate in 50 ml of methanol and 22 ml (44 mmol) of a 2 M solution of potassium carbonate in water, 1.18 g (95%) of the expected derivative are obtained in the form of an orange powder.

### l) Synthesis of 4-{2-[3-(benzylaminocarbonothioylhydrazono)-2-oxo-1-benzyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 36 mg of 4-benzylthiosemicarbazide in 2.5 ml of 10% acetic acid in ethanol and 0.5 ml (0.036 mmol) of a mixture of 195 mg of 4-[2-(2,3-dioxo-1-benzyl-2,3-dihydro-1H-indol-5-ylsulfanyl)ethyl]benzoic acid in 6.5 ml of 10% acetic acid in tetrahydrofuran, 20 mg of the expected derivative are obtained in the form of an orange solid.

### EXAMPLE 8

### Synthesis of 4-{2-[3-((anilinocarbonyl)hydrazono)-2-oxo-1-benzyl-2,3-dihydro-1H-indol-5-ylsulfanyl]ethyl}benzoic acid

Steps a), b), c), g), h), i), j) and k) are the same as those of Example 7.

### l) Synthesis of 4-{2-[3-((anilinocarbonyl)hydrazono)-2-oxo-1-benzyl-2,3-dihydro-1H-indol-5-ylsulfanyl]ethyl}benzoic acid

This step was performed by parallel chemistry in a 96-well plate.

In a manner similar to that of Example 7(l), by reacting 0.5 ml (0.040 mmol) of a solution of 30 mg of 4-phenylsemicarbazide in 2.5 ml of 10% acetic acid in ethanol and 0.5 ml (0.036 mmol) of a mixture of 195 mg of 4-[2-(2,3-dioxo-1-benzyl-2,3-dihydro-1H-indol-5-ylsulfanyl)ethyl]benzoic acid in 6.5 ml of 10% acetic acid in tetrahydrofuran, 14 mg of the expected derivative are obtained in the form of a golden-yellow solid.

### EXAMPLE 9

### Synthesis of 4-{2-[3-(anilinocarbonothioylhydrazono)-2-oxo-1-pentyl-2,3-dihydro-1H-indol-5-ylsulfanyl]ethyl}benzoic acid

Steps a), b), c), g), h), i), j) and k) are the same as those of Example 4.

### l) Synthesis of 4-{2-[3-(anilinocarbonothioylhydrazono)-2-oxo-1-pentyl-2,3-dihydro-1H-indol-5-ylsulfanyl]ethyl}benzoic acid

This step was performed by parallel chemistry.

In a manner similar to that of Example 3(l), by reacting 0.5 ml (0.040 mmol) of a solution of 6.6 mg of 4-phenyl-3-thiosemicarbazide in 0.5 ml of 10% acetic acid in ethanol and 0.5 ml (0.036 mmol) of a mixture of 31.5 mg of 4-[2-(2,3-dioxo-1-pentyl-2,3-dihydro-1H-indol-5-ylsulfanyl)ethyl]benzoic acid in 1.1 ml of 10% acetic acid in tetrahydrofuran, 17 mg of the expected derivative are obtained in the form of a dark orange solid.

### EXAMPLE 10

### Synthesis of 4-{2-[3-(ethylaminocarbonothioylhydrazono)-2-oxo-1-pentyl-2,3-dihydro-1H-indol-5-ylsulfanyl]ethyl}benzoic acid

Steps a), b), c), g), h), i), j) and k) are the same as those of Example 4.

### l) Synthesis of 4-{2-[3-(ethylaminocarbonothioylhydrazono)-2-oxo-1-pentyl-2,3-dihydro-1H-indol-5-ylsulfanyl]ethyl}benzoic acid

This step was performed by parallel chemistry.

In a manner similar to that of Example 3(l), by reacting 0.5 ml (0.040 mmol) of a solution of 4.7 mg of 4-ethyl-3-thiosemicarbazide in 0.5 ml of 10% acetic acid in ethanol and 0.5 ml (0.036 mmol) of a mixture of 31.5 mg of 4-[2-(2,3-dioxo-1-pentyl-2,3-dihydro-1H-indol-5-ylsulfanyl)ethyl]benzoic acid in 1.1 ml of 10% acetic acid in tetrahydrofuran, 17 mg of the expected derivative are obtained in the form of an orange solid.

### EXAMPLE 11

### Synthesis of methyl 4-{2-[3-((4-fluoroanilino)carbonylhydrazono)-2-oxo-1-phenethyl-2,3-dihydro-1H-indol-5-ylsulfanyl]ethyl}benzoate

### a) Preparation of 5-iodo-3,3-dimethoxy-1,3-dihydroindol-2-one

18.5 ml (0.34 mol) of concentrated sulfuric acid are added dropwise to a mixture of 16.5 g (0.06 mol) of 5-iodoisatin in 600 ml of methanol/trimethoxymethane (1:1). The reaction medium is then stirred at room temperature overnight. The reaction medium is poured into ice-water solution and neutralized to pH 8 with sodium hydrogen carbonate. The desired product is extracted with ethyl acetate. The organic phases are combined, washed with water, dried over magnesium sulfate and concentrated on a rotary evaporator under vacuum. The paste obtained is crystallized and washed with ethyl ether. After evaporating off the solvent, the expected compound (8.72 g; 50%) is isolated in the form of a pale orange solid.

### b) Preparation of 5-iodo-3,3-dimethoxy-1-phenethyl-1,3-dihydroindol-2-one

332 mg (8.3 mmol) of 60% sodium hydride are added portionwise to a mixture of 2.20 g (7.5 mmol) of 5-iodo-3,3-dimethoxy-1,3-dihydroindol-2-one in 25 ml of dimethylformamide. The reaction medium is stirred at room temperature for 1 hour 30 minutes. 1.55 ml (11.3 mmol) of 2-bromoethylbenzene dissolved in 3 ml of dimethylformamide are then added dropwise. The reaction medium is stirred at room temperature for 72 hours. The reaction medium is then poured into saturated ammonium chloride solution and extracted with ethyl acetate. The organic phases are combined, washed with water, dried over magnesium sulfate and concentrated on a rotary evaporator under vacuum. The product obtained (3.58 g) is purified by chromatography on a column of silica (dichloromethane). After evaporating off the solvent, the expected compound (2.07 g; 65%) is isolated in the form of a dark orange oil.

Steps c), g), h) are the same as those of Example 1.

### i) Preparation of methyl 4-[2-(3,3-dimethoxy-2-oxo-1-phenethyl-2,3-dihydro-1H-indol-5-ylsulfanyl)ethyl]benzoate

In a manner similar to that of Example 1(i), by reacting 2.83 g (6.75 mmol) of methyl 4-(2-mercaptoethyl)benzoate in 3 ml of tetrahydrofuran, 2.45 g (6.1 mmol) of polymer-supported borohydride resin Amberlite® IRA400 (2.5 mmol/g) (Aldrich: 32864-2), 26 mg of bis(bipyridine)nickel (II) bromide (Organometallics 1985, 4, 657-661) and 863 mg (2.0 mmol) of 5-iodo-3,3-dimethoxy-1-phenethyl-1,3-dihydroindol-2-one in 20 ml of ethanol, 800 mg (80%) of the expected derivative are obtained in the form of a yellowish oil.

### j) Preparation of methyl 4-[2-(2,3-dioxo-1-phenethyl-2,3-dihydro-1H-indol-5-ylsulfanyl)ethyl]benzoate

In a manner similar to that of Example 1(j), by reacting 9.5 ml (19 mmol) of 2N hydrochloric acid and 769 mg (1.56 mmol) of methyl 4-[2-(3,3-dimethoxy-2-oxo-1-phenethyl-2,3-dihydro-1H-indol-5-ylsulfanyl)ethyl]benzoate in 24 ml of acetone, 530 mg (76%) of the expected derivative are obtained in the form of a blood-red paste.

### l) Synthesis of methyl 4-{2-[3-((4-fluoroanilino)carbonylhydrazono)-2-oxo-1-phenethyl-2,3-dihydro-1H-indol-5-ylsulfanyl]ethyl}benzoate

A mixture of 8.1 mg (0.04 mmol) of 4-(4-fluorophenyl)semicarbazide hydrochloride and 16 mg (0.036 mmol) of methyl 4-[2-(2,3-dioxo-1-phenethyl-2,3-dihydro-1H-indol-5-ylsulfanyl)ethyl]benzoate in 1 ml of 10% acetic acid in methanol is stirred overnight. The precipitate obtained is filtered and dried, and 15.1 mg (70%) of the expected derivative are isolated in the form of a golden-yellow solid.

### EXAMPLE 12

### Synthesis of methyl 4-{2-[3-((anilinocarbonyl)hydrazono)-2-oxo-1-butyl-2,3-dihydro-1H-indol-5-ylsulfanyl]ethyl}benzoate

### a) Preparation of 5-iodo-3,3-dimethoxy-1,3-dihydroindol-2-one

18.5 ml (0.34 mol) of concentrated sulfuric acid are added dropwise to a mixture of 16.5 g (0.06 mol) of 5-iodoisatin in 600 ml of methanol/trimethoxymethane (1:1). The reaction medium is then stirred at room temperature overnight. The reaction medium is poured into ice-water solution and neutralized to pH 8 with sodium hydrogen carbonate. The desired product is extracted with ethyl acetate. The organic phases are combined, washed with water, dried over magnesium sulfate and concentrated on a rotary evaporator under vacuum. The paste obtained is crystallized and washed with ethyl ether. After evaporating off the solvent, the expected compound (8.72 g; 50%) is isolated in the form of a pale orange solid.

### b) Preparation of 5-iodo-3,3-dimethoxy-1-butyl-1,3-dihydroindol-2-one

332 mg (8.3 mmol) of 60% sodium hydride are added portionwise to a mixture of 2.20 g (7.5 mmol) of 5-iodo-3,3-dimethoxy-1,3-dihydroindol-2-one in 25 ml of dimethylformamide. The reaction medium is stirred at room temperature for 1 hour 30 minutes. 0.89 ml (8.3 mmol) of 1-bromobutane dissolved in 2 ml of dimethylformamide are then added dropwise. The reaction medium is stirred at room temperature overnight. The reaction medium is then poured into saturated ammonium chloride solution and extracted with ethyl acetate. The organic phases are combined, washed with water, dried over magnesium sulfate and concentrated on a rotary evaporator under vacuum. The product obtained (2.24 g) is purified by chromatography on a column of silica (dichloromethane). After evaporating off the solvent, the expected compound (1.64 g; 58%) is isolated in the form of a dark orange oil.

Steps c), g) and h) are the same as those of Example 11.

### i) Preparation of methyl 4-[2-(3,3-dimethoxy-2-oxo-1-butyl-2,3-dihydro-1H-indol-5-ylsulfanyl)ethyl]benzoate

In a manner similar to that of Example 1(i), by reacting 600 mg (3.06 mmol) of methyl 4-(2-mercaptoethyl)benzoate in 3 ml of tetrahydrofuran, 2.45 g (6.1 mmol) of polymer-supported borohydride resin Amberlite® IRA400 (2.5 mmol/g) (Aldrich: 32864-2), 26 mg of bis(bipyridine)nickel (II) bromide (Organometallics 1985, 4, 657-661) and 765 mg (2.0 mmol) of 5-iodo-3,3-dimethoxy-1-phenethyl-1,3-dihydroindol-2-one in 20 ml of ethanol, 391 mg (43%) of the expected derivative are obtained in the form of a yellow oil.

### j) Preparation of methyl 4-[2-(2,3-dioxo-1-butyl-2,3-dihydro-1H-indol-5-ylsulfanyl)ethyl]benzoate

In a manner similar to that of Example 1(j), by reacting 5.5 ml (11 mmol) of 2N hydrochloric acid and 371 mg (0.84 mmol) of methyl 4-[2-(3,3-dimethoxy-2-oxo-1-butyl-2,3-dihydro-1H-indol-5-ylsulfanyl)ethyl]benzoate in 13 ml of acetone, 240 mg (72%) of the expected derivative are obtained in the form of a blood-red paste.

### l) Synthesis of methyl 4-{2-[3-((anilinocarbonyl)hydrazono)-2-oxo-1-butyl-2,3-dihydro-1H-indol-5-ylsulfanyl]ethyl}benzoate

This step was performed by parallel chemistry in a 96-well plate.

In a manner similar to that of Example 3(l), by reacting 0.5 ml (0.040 mmol) of a solution of 39 mg of 4-phenylsemicarbazide in 3.25 ml of 10% acetic acid in methanol and 0.5 ml (0.036 mmol) of a solution of 64 mg of methyl 4-[2-(2,3-dioxo-1-butyl-2,3-dihydro-1H-indol-5-ylsulfanyl)ethyl]benzoate in 2.25 ml of 10% acetic acid in methanol, 14 mg of the expected derivative are obtained.

### EXAMPLE 13

### Synthesis of methyl 4-{2-[3-((anilinocarbonyl)hydrazono)-2-oxo-1-heptyl-2,3-dihydro-1H-indol-5-ylsulfanyl]ethyl}benzoate

Step a) is performed in a manner similar to that of Example 12.

### b) Preparation of 5-iodo-3,3-dimethoxy-1-heptyl-1,3-dihydroindol-2-one

In a manner similar to that of Example 12(b), by reacting 332 mg (8.3 mmol) of 60% sodium hydride, 2.20 g (7.5 mmol) of 5-iodo-3,3-dimethoxy-1,3-dihydroindol-2-one in 25 ml of dimethylformamide and 1.30 ml (8.3 mmol) of 1-bromoheptane dissolved in 3 ml of dimethylformamide, 1.92 g (61%) of the expected derivative are obtained in the form of a dark orange oil.

Steps c), g) and h) are the same as those of Example 11.

### i) Preparation of methyl 4-[2-(3,3-dimethoxy-2-oxo-1-heptyl-2,3-dihydro-1H-indol-5-ylsulfanyl)ethyl]benzoate

In a manner similar to that of Example 1(i), by reacting 600 mg (3.06 mmol) of methyl 4-(2-mercaptoethyl)benzoate in 3 ml of tetrahydrofuran, 2.45 g (6.1 mmol) of polymer-supported borohydride resin Amberlite® IRA400 (2.5 mmol/g) (Aldrich: 32864-2), 26 mg of bis(bipyridine)nickel (II) bromide (Organometallics 1985, 4, 657-661) and 851 mg (2.0 mmol) of 5-iodo-3,3-dimethoxy-1-phenethyl-1,3-dihydroindol-2-one in 20 ml of ethanol, 416 mg (41%) of the expected derivative are obtained in the form of a yellowish oil.

### j) Preparation of methyl 4-[2-(2,3-dioxo-1-heptyl-2,3-dihydro-1H-indol-5-ylsulfanyl)ethyl]benzoate

In a manner similar to that of Example 1(j), by reacting 5.0 ml (10 mmol) of 2N hydrochloric acid and 400 mg (0.80 mmol) of methyl 4-[2-(3,3-dimethoxy-2-oxo-1-heptyl-2,3-dihydro-1H-indol-5-ylsulfanyl)ethyl]benzoate in 12 ml of acetone, 290 mg (82%) of the expected derivative are obtained in the form of a blood-red paste.

### l) Synthesis of methyl 4-{2-[3-((anilinocarbonyl)hydrazono)-2-oxo-1-heptyl-2,3-dihydro-1H-indol-5-ylsulfanyl]ethyl}benzoate

This step was performed by parallel chemistry in a 96-well plate.

In a manner similar to that of Example 3(l), by reacting 0.5 ml (0.040 mmol) of a solution of 39 mg of 4-phenylsemicarbazide in 3.25 ml of 10% acetic acid in methanol and 0.5 ml (0.036 mmol) of a solution of 71 mg of methyl 4-[2-(2,3-dioxo-1-heptyl-2,3-dihydro-1H-indol-5-ylsulfanyl)ethyl]benzoate in 2.25 ml of 1 0% acetic acid in methanol, 16 mg of the expected derivative are obtained.

### EXAMPLE 14

### Synthesis of 4-{2-[3-((anilinocarbonyl)hydrazono)-2-oxo-1-butyl-2,3-dihydro-1H-indol-5-ylsulfanyl]ethyl}benzoic acid

Steps a), b), c), g), h), i) and j) are the same as those of Example 12.

### k) Preparation of 4-[2-(2,3-dioxo-1-butyl-2,3-dihydro-1H-indol-5-ylsulfanyl)ethyl]benzoic acid

In a manner similar to that of Example 1(k), by reacting 150 mg (0.38 mmol) of methyl 4-[2-(2,3-dioxo-1-butyl-2,3-dihydro-1H-indol-5-ylsulfanyl)ethyl]benzoate in 8 ml of methanol and 3 ml (6 mmol) of a 2 M solution of potassium carbonate in water, 120 mg (82%) of the expected derivative are obtained in the form of an orange-beige powder.

### l) Synthesis of 4-{2-[3-((anilinocarbonyl)hydrazono)-2-oxo-1-butyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 39 mg of 4-phenylsemicarbazide in 3.25 ml of 10% acetic acid in methanol and 0.5 ml (0.036 mmol) of a solution of 104 mg of 4-[2-(2,3-dioxo-1-butyl-2,3-dihydro-1H-indol-5-ylsulfanyl)ethyl]benzoic acid in 3.75 ml of 10% acetic acid in methanol, 13 mg of the expected derivative are obtained.

### EXAMPLE 15

### Synthesis of 4-{2-[3-((anilinocarbonyl)hydrazono)-2-oxo-1-heptyl-2,3-dihydro-1H-indol-5-ylsulfanyl]ethyl}benzoic acid

Steps a), b), c), g), h), i) and j) are the same as those of Example 13.

### k) Preparation of 4-[2-(2,3-dioxo-1-heptyl-2,3-dihydro-1H-indol-5-ylsulfanyl)ethyl]benzoic acid

In a manner similar to that of Example 1(k), by reacting 200 mg (0.45 mmol) of methyl 4-[2-(2,3-dioxo-1-heptyl-2,3-dihydro-1H-indol-5-ylsulfanyl)ethyl]benzoate in 10 ml of methanol and 3.3 ml (6.6 mmol) of a 2 M solution of potassium carbonate in water, 175 mg (91%) of the expected derivative are obtained in the form of a dark orange powder.

### l) Synthesis of 4-{2-[3-((anilinocarbonyl)hydrazono)-2-oxo-1-heptyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 39 mg of 4-phenylsemicarbazide in 3.25 ml of 10% acetic acid in methanol and 0.5 ml (0.036 mmol) of a solution of 161 mg of 4-[2-(2,3-dioxo-1-heptyl-2,3-dihydro-1H-indol-5-ylsulfanyl)ethyl]benzoic acid in 5.25 ml of 10% acetic acid in methanol, 17 mg of the expected derivative are obtained.

### EXAMPLE 16

### Synthesis of 4-{2-[3-((anilinocarbonyl)hydrazono)-2-oxo-1-phenethyl-2,3-dihydro-1H-indol-5-ylsulfanyl]ethyl}benzoic acid

Steps a), b), c), g), h), i) and j) are the same as those of Example 11.

### k) Preparation of 4-[2-(2,3-dioxo-1-phenethyl-2,3-dihydro-1H-indol-5-ylsulfanyl)ethyl]benzoic acid

In a manner similar to that of Example 1(k), by reacting 260 mg (0.58 mmol) of methyl 4-[2-(2,3-dioxo-1-phenethyl-2,3-dihydro-1H-indol-5-ylsulfanyl)ethyl]benzoate in 15 ml of methanol and 4.5 ml (9 mmol) of a 2 M solution of potassium carbonate in water, 160 mg (64%) of the expected derivative are obtained in the form of an orange powder.

### l) Synthesis of 4-{2-[3-((anilinocarbonyl)hydrazono)-2-oxo-1-phenethyl-2,3-dihydro-1H-indol-5-ylsulfanyl]ethyl)benzoic acid

This step was performed by parallel chemistry in a 96-well plate.

In a manner similar to that of Example 3(l), by reacting 0.5 ml (0.040 mmol) of a solution of 39 mg of 4-phenylsemicarbazide in 3.25 ml of 10% acetic acid in methanol and 0.5 ml (0.036 mmol) of a solution of 148 mg of 4-[2-(2,3-dioxo-1-phenethyl-2,3-dihydro-1H-indol-5-ylsulfanyl)ethyl]benzoic acid in 4.75 ml of 10% acetic acid in methanol, 15 mg of the expected derivative are obtained.

### EXAMPLE 17

### Synthesis of methyl 4-{2-[3-((3-fluoroanilinocarbonyl)hydrazono)-2-oxo-1-phenethyl-2,3-dihydro-1H-indol-5-ylsulfanyl]ethyl}benzoate

Steps a), b), c), g), h), i) and j) are the same as those of Example 11.

### l) Synthesis of methyl 4-{2-[3-((3-fluoroanilinocarbonyl)hydrazono)-2-oxo-1-phenethyl-2,3-dihydro-1H-indol-5-ylsulfanyl]ethyl}benzoate

This step was performed by parallel chemistry in a 96-well plate.

In a manner similar to that of Example 3(l), by reacting 0.5 ml (0.040 mmol) of a solution of 30 mg of 4-(3-fluorophenyl)semicarbazide in 2.25 ml of 10% acetic acid in methanol and 0.5 ml (0.036 mmol) of a solution of 184.5 mg of methyl 4-[2-(2,3-dioxo-1-phenethyl-2,3-dihydro-1H-indol-5-ylsulfanyl)ethyl]benzoate in 5.75 ml of 10% acetic acid in methanol, 16 mg of the expected derivative are obtained.

### EXAMPLE 18

### Synthesis of 4-{2-[3-((3-fluoroanilinocarbonyl)hydrazono)-2-oxo-1-butyl-2,3-dihydro-1H-indol-5-ylsulfanyl]ethyl}benzoic acid

Steps a), b), c), g), h), i), j) and k) are the same as those of Example 14.

### l) Synthesis of 4-{2-[3-((3-fluoroanilinocarbonyl)hydrazono)-2-oxo-1-butyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 30 mg of 4-(3-fluorophenyl)semicarbazide in 2.25 ml of 10% acetic acid in methanol and 0.5 ml (0.036 mmol) of a solution of 104 mg of 4-[2-(2,3-dioxo-1-butyl-2,3-dihydro-1H-indol-5-ylsulfanyl)ethyl]benzoic acid in 3.75 ml of 10% acetic acid in methanol, 12 mg of the expected derivative are obtained.

### EXAMPLE 19

### Synthesis of methyl 4-{2-[3-((3-trifluoromethylanilinocarbonyl)hydrazono)-2-oxo-1-phenethyl-2,3-dihydro-1H-indol-5-ylsulfanyl]ethyl}benzoate

Steps a), b), c), g), h), i) and j) are the same as those of Example 11.

### l) Synthesis of methyl 4-{2-[3-((3-trifluoromethylanilinocarbonyl)hydrazono)-2-oxo-1-phenethyl-2,3-dihydro-1H-indol-5-ylsulfanyl]ethyl}benzoate

This step was performed by parallel chemistry in a 96-well plate.

In a manner similar to that of Example 3(l), by reacting 0.5 ml (0.040 mmol) of a solution of 25 mg of 4-[3-(trifluoromethyl)phenyl]semicarbazide hydrochloride in 1.25 ml of 10% acetic acid in methanol and 0.5 ml (0.036 mmol) of a solution of 184.5 mg of methyl 4-[2-(2,3-dioxo-1-phenethyl-2,3-dihydro-1H-indol-5-ylsulfanyl)ethyl]benzoate in 5.75 ml of 10% acetic acid in methanol, 12 mg of the expected derivative are obtained.

### EXAMPLE 20

### Synthesis of 4-{2-[3-((3-trifluoromethylanilinocarbonyl)hydrazono)-2-oxo-1-heptyl-2,3-dihydro-1H-indol-5-ylsulfanyl]ethyl}benzoic acid

Steps a), b), c), g), h), i), j) and k) are the same as those of Example 15.

### l) Synthesis of 4-{2-[3-((3-trifluoromethylanilinocarbonyl)hydrazono)-2-oxo-1-heptyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 25 mg of 4-[3-(trifluoromethyl)phenyl]semicarbazide hydrochloride in 1.25 ml of 10% acetic acid in methanol and 0.5 ml (0.036 mmol) of a solution of 161 mg of 4-[2-(2,3-dioxo-1-heptyl-2,3-dihydro-1H-indol-5-ylsulfanyl)ethyl]benzoic acid in 5.25 ml of 10% acetic acid in methanol, 17 mg of the expected derivative are obtained.

### EXAMPLE 21

### Synthesis of methyl 4-{2-[3((2-chloroanilinocarbonyl)hydrazono)-2-oxo-1-phenethyl-2,3-dihydro-1H-indol-5-ylsulfanyl]ethyl}benzoate

Steps a), b), c), g), h), i) and j) are the same as those of Example 11.

### l) Synthesis of methyl 4-{2-[3-((2-chloroanilinocarbonyl)hydrazono)-2-oxo-1-phenethyl-2,3-dihydro-1H-indol-5-ylsulfanyl]ethyl}benzoate

This step was performed by parallel chemistry in a 96-well plate.

In a manner similar to that of Example 3(l), by reacting 0.5 ml (0.040 mmol) of a solution of 31 mg of 4-(2-chlorophenyl)semicarbazide hydrochloride in 1.75 ml of 10% acetic acid in methanol and 0.5 ml (0.036 mmol) of a solution of 184.5 mg of methyl 4-[2-(2,3-dioxo-1-phenethyl-2,3-dihydro-1H-indol-5-ylsulfanyl)ethyl]benzoate in 5.75 ml of 10% acetic acid in methanol, 4.2 mg of the expected derivative are obtained.

### EXAMPLE 22

### Synthesis of 4-{2-[3((2-chloroanilinocarbonyl)hydrazono)-2-oxo-1-heptyl-2,3-dihydro-1H-indol-5-ylsulfanyl]ethyl}benzoic acid

Steps a), b), c), g), h), i), j) and k) are the same as those of Example 15.

### l) Synthesis of 4-{2-[3((2-chloroanilinocarbonyl)hydrazono)-2-oxo-1-heptyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 31 mg of 4-(2-chlorophenyl)semicarbazide hydrochloride in 1.75 ml of 10% acetic acid in methanol and 0.5 ml (0.036 mmol) of a solution of 161 mg of 4-[2-(2,3-dioxo-1-heptyl-2,3-dihydro-1H-indol-5-ylsulfanyl)ethyl]benzoic acid in 5.25 ml of 10% acetic acid in methanol, 17 mg of the expected derivative are obtained.

### EXAMPLE 23

### Synthesis of 4-{2-[3((2-chloroanilinocarbonyl)hydrazono)-2-oxo-1-phenethyl-2,3-dihydro-1H-indol-5-ylsulfanyl]ethyl}benzoic acid

Steps a), b), c), g), h), i), j) and k) are the same as those of Example 16.

### l) Synthesis of 4-{2-[3((2-chloroanilinocarbonyl)hydrazono)-2-oxo-1-phenethyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 31 mg of 4-(2-chlorophenyl)semicarbazide hydrochloride in 1.75 ml of 10% acetic acid in methanol and 0.5 ml (0.036 mmol) of a solution of 148 mg of 4-[2-(2,3-dioxo-1-phenethyl-2,3-dihydro-1H-indol-5-ylsulfanyl)ethyl]benzoic acid in 4.75 ml of 10% acetic acid in methanol, 18 mg of the expected derivative are obtained.

### EXAMPLE 24

### Synthesis of 4-{2-[3((4-fluoroanilinocarbonyl)hydrazono)-2-oxo-1-heptyl-2,3-dihydro-1H-indol-5-ylsulfanyl]ethyl}benzoic acid

Steps a), b), c), g), h), i), j) and k) are the same as those of Example 15.

### l) Synthesis of 4-{2-[3((4-fluoroanilinocarbonyl)hydrazono)-2-oxo-1-heptyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 28.5 mg of 4-(4-fluorophenyl)semicarbazide hydrochloride in 1.75 ml of 10% acetic acid in methanol and 0.5 ml (0.036 mmol) of a solution of 161 mg of 4-[2-(2,3-dioxo-1-heptyl-2,3-dihydro-1H-indol-5-ylsulfanyl)ethyl]benzoic acid in 5.25 ml of 10% acetic acid in methanol, 16 mg of the expected derivative are obtained.

### EXAMPLE 25

### Synthesis of 4-{2-[3((4-fluoroanilinocarbonyl)hydrazono)-2-oxo-1-phenethyl-2,3-dihydro-1H-indol-5-ylsulfanyl]ethyl}benzoic acid

Steps a), b), c), g), h), i), j) and k) are the same as those of Example 16.

### l) Synthesis of 4-{2-[3((4-fluoroanilinocarbonyl)hydrazono)-2-oxo-1-phenethyl-2,3-dihydro-1H-indol-5-ylsulfanyl]ethyl)benzoic acid

This step was performed by parallel chemistry in a 96-well plate.

In a manner similar to that of Example 3(l), by reacting 0.5 ml (0.040 mmol) of a solution of 28.5 mg of 4-(4-fluorophenyl)semicarbazide hydrochloride in 1.75 ml of 10% acetic acid in methanol and 0.5 ml (0.036 mmol) of a solution of 148 mg of 4-[2-(2,3-dioxo-1-phenethyl-2,3-dihydro-1H-indol-5-ylsulfanyl)ethyl]benzoic acid in 4.75 ml of 10% acetic acid in methanol, 19 mg of the expected derivative are obtained.

### EXAMPLE 26

### Synthesis of 4-{2-[3((4-methylbenzenesulfonamido)carbonylhydrazono)-2-oxo-1-phenethyl-2,3-dihydro-1H-indol-5-ylsulfanyl]ethyl}benzoic acid

Steps a), b), c), g), h), i), j) and k) are the same as those of Example 16.

### l) Synthesis of 4-{2-[3((4-methylbenzenesulfonamido)carbonylhydrazono)-2-oxo-1-phenethyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 47.4 mg of 4-(4-methylphenylsulfonyl)semicarbazide hydrochloride in 2.25 ml of 10% acetic acid in methanol and 0.5 ml (0.036 mmol) of a solution of 148 mg of 4-[2-(2,3-dioxo-1-phenethyl-2,3-dihydro-1H-indol-5-ylsulfanyl)ethyl]benzoic acid in 4.75 ml of 10% acetic acid in methanol, 11 mg of the expected derivative are obtained.

### EXAMPLE 27

### Synthesis of methyl 4-{2-[3((3,4-dichloroanilinocarbonyl)hydrazono)-2-oxo-1-1-phenethyl-2,3-dihydro-1H-indol-5-ylsulfanyl]ethyl}benzoate

Steps a), b), c), g), h), i) and j) are the same as those of Example 11.

### l) Synthesis of methyl 4-{2-[3((3,4-dichloroanilinocarbonyl)hydrazono)-2-oxo-1-1-phenethyl-2,3-dihydro-1H-indol-5-ylsulfanyl]ethyl}benzoate

This step was performed by parallel chemistry in a 96-well plate.

In a manner similar to that of Example 3(l), by reacting 0.5 ml (0.040 mmol) of a solution of 15.2 mg of 4-(3,4-dichlorophenyl)semicarbazide hydrochloride in 0.75 ml of 10% acetic acid in methanol and 0.5 ml (0.036 mmol) of a solution of 184.5 mg of methyl 4-[2-(2,3-dioxo-1-phenethyl-2,3-dihydro-1H-indol-5-ylsulfanyl)ethyl]benzoate in 5.75 ml of 10% acetic acid in methanol, 23 mg of the expected derivative are obtained.

### EXAMPLE 28

### Synthesis of methyl 4-(2-{1-butyl-3-[(morpholine-4-carbonyl)hydrazono]-2-oxo-2,3-dihydro-1H-indol-5-ylsulfanyl}ethyl)benzoate

Steps a), b), c), g), h), i) and j) are the same as those of Example 12.

### l) Synthesis of methyl 4-(2-{1-butyl-3-[(morpholine-4-carbonyl)hydrazono]-2-oxo-2,3-dihydro-1H-indol-5-ylsulfanyl}ethyl)benzoate

This step was performed by parallel chemistry in a 96-well plate.

In a manner similar to that of Example 3(l), by reacting 0.5 ml (0.040 mmol) of a solution of 37 mg of morpholine-4-carbohydrazide in 3.25 ml of 10% acetic acid in methanol and 0.5 ml (0.036 mmol) of a solution of 64 mg of methyl 4-[2-(2,3-dioxo-1-butyl-2,3-dihydro-1H-indol-5-ylsulfanyl)ethyl]benzoate in 2.25 ml of 10% acetic acid in methanol, 8 mg of the expected derivative are obtained.

### EXAMPLE 29

### Synthesis of methyl 4-(2-{1-Heptyl-3-[(morpholine-4-carbonyl)hydrazono]-2-oxo-2,3-dihydro-1H-indol-5-ylsulfanyl}ethyl)benzoate

Steps a), b), c), g), h), i) and j) are the same as those of Example 13.

### l) Synthesis of methyl 4-(2-{1-heptyl-3-[(morpholine-4-carbonyl)hydrazono]-2-oxo-2,3-dihydro-1H-indol-5-ylsulfanyl}ethyl)benzoate

This step was performed by parallel chemistry in a 96-well plate.

In a manner similar to that of Example 3(l), by reacting 0.5 ml (0.040 mmol) of a solution of 37 mg of morpholine-4-carbohydrazide in 3.25 ml of 10% acetic acid in methanol and 0.5 ml (0.036 mmol) of a solution of 71 mg of methyl 4-[2-(2,3-dioxo-1-heptyl-2,3-dihydro-1H-indol-5-ylsulfanyl)ethyl]benzoate in 2.25 ml of 10% acetic acid in methanol, 13 mg of the expected derivative are obtained.

### EXAMPLE 30

### Synthesis of methyl 4-(2-{3-[(morpholine-4-carbonyl)hydrazono]-2-oxo-1-phenethyl-2,3-dihydro-1H-indol-5-ylsulfanyl}ethyl)benzoate

Steps a), b), c), g), h), i) and j) are the same as those of Example 11.

### l) Synthesis of methyl 4-(2-{3-[(morpholine-4-carbonyl)hydrazono]-2-oxo-1-phenethyl-2,3-dihydro-1H-indol-5-ylsulfanyl}ethyl)benzoate

This step was performed by parallel chemistry in a 96-well plate.

In a manner similar to that of Example 3(l), by reacting 0.5 ml (0.040 mmol) of a solution of 37 mg of morpholine-4-carbohydrazide in 3.25 ml of 10% acetic acid in methanol and 0.5 ml (0.036 mmol) of a solution of 184.5 mg of methyl 4-[2-(2,3-dioxo-1-phenethyl-2,3-dihydro-1H-indol-5-ylsulfanyl)ethyl]benzoate in 5.75 ml of 10% acetic acid in methanol, 15 mg of the expected derivative are obtained.

### EXAMPLE 31

### Synthesis of 4-(2-{1-butyl-3-[(morpholine-4-carbonyl)hydrazono]-2-oxo-2,3-dihydro-1H-indol-5-ylsulfanyl}ethyl)benzoic acid

Steps a), b), c), g), h), i), j) and k) are the same as those of Example 14.

### l) Synthesis of 4-(2-{1-butyl-3-[(morpholine-4-carbonyl)hydrazono]-2-oxo-2,3-dihydro-1H-indol-5-ylsulfanyl}ethyl)benzoic acid

This step was performed by parallel chemistry in a 96-well plate.

In a manner similar to that of Example 3(l), by reacting 0.5 ml (0.040 mmol) of a solution of 37 mg of morpholine-4-carbohydrazide in 3.25 ml of 10% acetic acid in methanol and 0.5 ml (0.036 mmol) of a solution of 104 mg of 4-[2-(2,3-dioxo-1-butyl-2,3-dihydro-1H-indol-5-ylsulfanyl)ethyl]benzoic acid in 3.75 ml of 10% acetic acid in methanol, 10 mg of the expected derivative are obtained.

### EXAMPLE 32

### Synthesis of 4-(2-{1-heptyl-3-[(morpholine-4-carbonyl)hydrazono]-2-oxo-2,3-dihydro-1H-indol-5-ylsulfanyl}ethyl)benzoic acid

Steps a), b), c), g), h), i), j) and k) are the same as those of Example 15.

### l) Synthesis of 4-(2-{1-heptyl-3-[(morpholine-4-carbonyl)hydrazono]-2-oxo-2,3-dihydro-1H-indol-5-ylsulfanyl}ethyl)benzoic acid

This step was performed by parallel chemistry in a 96-well plate.

In a manner similar to that of Example 3(l), by reacting 0.5 ml (0.040 mmol) of a solution of 37 mg of morpholine-4-carbohydrazide in 3.25 ml of 10% acetic acid in methanol and 0.5 ml (0.036 mmol) of a solution of 161 mg of 4-[2-(2,3-dioxo-1-heptyl-2,3-dihydro-1H-indol-5-ylsulfanyl)ethyl]benzoic acid in 5.25 ml of 10% acetic acid in methanol, 11.5 mg of the expected derivative are obtained.

### EXAMPLE 33

### Synthesis of 4-(2-{3-[(morpholine-4-carbonyl)hydrazono]-2-oxo-1-phenethyl-2,3-dihydro-1H-indol-5-ylsulfanyl}ethyl)benzoic acid

Steps a), b), c), g), h), i), j) and k) are the same as those of Example 16.

### l) Synthesis of 4-(2-{3-[(morpholine-4-carbonyl)hydrazono]-2-oxo-1-phenethyl-2,3-dihydro-1H-indol-5-ylsulfanyl}ethyl)benzoic acid

This step was performed by parallel chemistry in a 96-well plate.

In a manner similar to that of Example 3(l), by reacting 0.5 ml (0.040 mmol) of a solution of 37 mg of morpholine-4-carbohydrazide in 3.25 ml of 10% acetic acid in methanol and 0.5 ml (0.036 mmol) of a solution of 148 mg of 4-[2-(2,3-dioxo-1-phenethyl-2,3-dihydro-1H-indol-5-ylsulfanyl)ethyl]benzoic acid in 4.75 ml of 10% acetic acid in methanol, 8.3 mg of the expected derivative are obtained.

### EXAMPLE 34

### Synthesis of methyl 4-{2-[3-((2-chloro-4-trifluoromethylanilinocarbonyl)hydrazono)-2-oxo-1-phenethyl-2,3-dihydro-1H-indol-5-ylsulfanyl]ethyl}benzoate

Steps a), b), c), g), h), i) and j) are the same as those of Example 11.

### l) Synthesis of methyl 4-{2-[3-((2-chloro-4-trifluoromethylanilinocarbonyl)hydrazono)-2-oxo-1-phenethyl-2,3-dihydro-1H-indol-5-ylsulfanyl]ethyl}benzoate

This step was performed by parallel chemistry in a 96-well plate.

In a manner similar to that of Example 3(l), by reacting 0.5 ml (0.040 mmol) of a solution of 35 mg of N1-[2-chloro-4-(trifluoromethyl)phenyl]hydrazine-1-carboxamide in 1.75 ml of 10% acetic acid in methanol and 0.5 ml (0.036 mmol) of a solution of 184.5 mg of methyl 4-[2-(2,3-dioxo-1-phenethyl-2,3-dihydro-1H-indol-5-ylsulfanyl)ethyl]benzoate in 5.75 ml of 10% acetic acid in methanol, 17 mg of the expected derivative are obtained.

### EXAMPLE 35

### Synthesis of 4-{2-[3((2-chloro-4-trifluoromethylanilinocarbonyl)hydrazono)-2-oxo-1-phenethyl-2,3-dihydro-1H-indol-5-ylsulfanyl]ethyl}benzoic acid

Steps a), b), c), g), h), i), j) and k) are the same as those of Example 16.

### l) Synthesis of 4-{2-[3((2-chloro-4-trifluoromethylanilinocarbonyl)hydrazono)-2-oxo-1-phenethyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 35 mg of N1-[2-chloro-4-(trifluoromethyl)phenyl]hydrazine-1-carboxamide in 1.75 ml of 10% acetic acid in methanol and 0.5 ml (0.036 mmol) of a solution of 148 mg of 4-[2-(2,3-dioxo-1-phenethyl-2,3-dihydro-1H-indol-5-ylsulfanyl)ethyl]benzoic acid in 4.75 ml of 10% acetic acid in methanol, 18 mg of the expected derivative are obtained.

### EXAMPLE 36

### Synthesis of methyl 4-{2-[3((tert-butylamino)carbonylhydrazono)-2-oxo-1-phenethyl-2,3-dihydro-1H-indol-5-ylsulfanyl]ethyl}benzoate

Steps a), b), c), g), h), i) and j) are the same as those of Example 11.

### l) Synthesis of methyl 4-{2-[3((tert-butylamino)carbonylhydrazono)-2-oxo-1-phenethyl-2,3-dihydro-1H-indol-5-ylsulfanyl]ethyl}benzoate

This step was performed by parallel chemistry in a 96-well plate.

In a manner similar to that of Example 3(l), by reacting 0.5 ml (0.040 mmol) of a solution of 131 mg of N1-(tert-butyl)hydrazine-1-carboxamide in 3.25 ml of 10% acetic acid in methanol and 0.5 ml (0.036 mmol) of a solution of 184.5 mg of methyl 4-[2-(2,3-dioxo-1-phenethyl-2,3-dihydro-1H-indol-5-ylsulfanyl)ethyl]benzoate in 5.75 ml of 10% acetic acid in methanol, 10 mg of the expected derivative are obtained.

### EXAMPLE 37

### Synthesis of ethyl 4-{2-[3(((anilinocarbonyl)hydrazono)-2-oxo-1-pentyl-2,3-dihydro-1H-indol-5-ylsulfanyl]ethyl}benzoate

Steps a), b), c), g), h), i) and j) are the same as those of Example 1.

### l) Synthesis of ethyl 4-{2-[3(((anilinocarbonyl)hydrazono)-2-oxo-1-pentyl-2,3-dihydro-1H-indol-5-ylsulfanyl]ethyl}benzoate

In a manner similar to that of Example 3(l), by reacting 0.5 ml (0.040 mmol) of a solution of 20 mg of 4-phenylsemicarbazide in 1.75 ml of 10% acetic acid in ethanol and 12.7 mg (0.036 mmol) of ethyl 4-[2-(2,3-dioxo-1-pentyl-2,3-dihydro-1H-indol-5-ylsulfanyl)ethyl]benzoate in 0.5 ml of 10% acetic acid in ethanol, 20 mg of the expected derivative are obtained.

### EXAMPLE 38

### Synthesis of 4-{2-[3-((3-fluoroanilinocarbonyl)hydrazono)-2-oxo-1-heptyl-2,3-dihydro-1H-indol-5-ylsulfanyl]ethyl}benzoic acid

Steps a), b), c), g), h), i), j) and k) are the same as those of Example 15.

### l) Synthesis of 4-{2-[3-((3-fluoroanilinocarbonyl)hydrazono)-2-oxo-1-heptyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 30 mg of 4-(3-fluorophenyl)semicarbazide in 2.25 ml of 10% acetic acid in methanol and 0.5 ml (0.036 mmol) of a solution of 161 mg of 4-[2-(2,3-dioxo-1-heptyl-2,3-dihydro-1H-indol-5-ylsulfanyl)ethyl]benzoic acid in 5.25 ml of 10% acetic acid in methanol, 16 mg of the expected derivative are obtained.

### EXAMPLE 39

### Synthesis of 4-{2-[3((aminocarbonyl)hydrazono)-2-oxo-1-pentyl-2,3-dihydro-1H-indol-5-ylsulfanyl]ethyl}benzoic acid

Steps a), b), c), g), h), i), j) and k) are the same as those of Example 1.

### l) Synthesis of 4-{2-[3((aminocarbonyl)hydrazono)-2-oxo-1-pentyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 31 mg of semicarbazide hydrochloride in 3.5 ml of 10% acetic acid in ethanol and 1 ml (0.036 mmol) of a solution of 157 mg of 4-[2-(2,3-dioxo-1-pentyl-2,3-dihydro-1H-indol-5-ylsulfanyl)ethyl]benzoic acid in 11 ml of 10% acetic acid in ethanol/tetrahydrofuran (1:1), and after filtering off the precipitate, 11 mg of the expected derivative are obtained in the form of a yellow solid.

### EXAMPLE 40

### Synthesis of 4-[3-(3-((3-trifluoromethylanilinocarbonyl)hydrazono)-2-oxo-1-propyl-2,3-dihydro-1H-indol-5-ylsulfanyl)propyl]benzoic acid

### a) Preparation of 5-iodo-1-propyl-1H-indole-2,3-dione

720 mg (0.018 mol) of 60% sodium hydride are added portionwise to a mixture of 4.10 g (0.015 mol) of 5-iodoisatin in 40 ml of dimethylformamide. The reaction medium is stirred at room temperature for 2 hours. 2.23 g (0.018 mol) of 1-bromopropane dissolved in 4 ml of dimethylformamide are then added dropwise. The reaction medium is stirred at room temperature overnight. The reaction medium is then poured into saturated ammonium chloride solution and extracted with ethyl acetate. The organic phases are combined, washed with water, dried over magnesium sulfate and concentrated on a rotary evaporator under vacuum. The solid is taken up in heptane, filtered and dried. 4.09 g (87%) of the expected compound are obtained in the form of a carmine-red powder.

### b) Preparation of 5-iodo-3,3-dimethoxy-1-propyl-1,3-dihydroindol-2-one

4.2 ml (0.077 mol) of concentrated sulfuric acid are added dropwise to a mixture of 4.07 g (0.0129 mol) of 5-iodo-1-propyl-1H-indole-2,3-dione in 128 ml of methanol/trimethoxymethane (1:1). The reaction medium is then stirred at room temperature overnight. The reaction medium is poured into ice-water solution and neutralized to pH 8 with sodium hydrogen carbonate. The desired product is extracted with ethyl ether. The organic phases are combined, washed with water, dried over magnesium sulfate and concentrated on a rotary evaporator under vacuum. The oil obtained (5.05 g) is purified by chromatography on a column of silica (dichloromethane). After evaporating off the solvent, the expected compound (4.86 g; 100%) is isolated in the form of an orange oil.

### d) Preparation of ethyl 4-(3-oxopropyl)benzoate

A mixture of 150 g (0.54 mol) of ethyl 4-iodobenzoate, 47 g (0.81 mol) of allyl alcohol, 113.4 g (1.35 mol) of potassium hydrogen carbonate, 3.65 g (0.016 mol) of palladium acetate and 174 g (0.54 mol) of tetrabutylammonium bromide in 1875 ml of dimethylformamide is stirred at room temperature for 4 days. The reaction medium is filtered through Celite and then poured into water and extracted with ethyl acetate. The organic phases are combined, washed with water, dried over magnesium sulfate and concentrated on a rotary evaporator under vacuum. The black oil obtained (188 g) is purified by chromatography on a column of silica (dichloromethane). After evaporating off the solvents, the expected compound (94 g; 84%) is isolated in the form of an orange-yellow oil.

### e) Preparation of ethyl 4-(3-hydroxypropyl)benzoate

94 g (0.455 mol) of ethyl 4-(3-oxopropyl)benzoate in 700 ml of methanol are added over 1 hour 40 minutes to a mixture, cooled to 0-5° C., of 26 g (0.683 mol) of 5-iodo-3,3-dimethoxy-1,3-dihydroindol-2-one in 25 ml of dimethylformamide. Water is then poured into the reaction medium, which is extracted with ethyl acetate. The organic phases are combined, washed with water, dried over magnesium sulfate and concentrated on a rotary evaporator under vacuum. The expected compound (91 g; 96%) is isolated in the form of a dark yellow oil.

### f) Preparation of ethyl 4-(3-bromopropyl)benzoate

160 g (0.612 mol) of triphenylphosphine are added to a mixture of 91 g (0.437 mol) of ethyl 4-(3-hydroxypropyl)benzoate in 600 ml of dichloromethane, followed by addition of 203 g (0.612 mol) of carbon tetrabromide. The reaction medium is stirred at room temperature for 30 minutes. Water is then poured into the reaction medium, which is extracted with ethyl acetate. The organic phases are combined and concentrated on a rotary evaporator under vacuum. The precipitate (triphenyl phosphite salt) is filtered off and washed, and the filtrate is concentrated. The orange oil obtained is purified by chromatography on a column of silica (95/5 heptane/ethyl acetate). After evaporating off the solvents, the expected compound (150 g; >100%) is isolated in the form of a yellow oil.

### g) Preparation of ethyl 4-(3-acetylsulfanylpropyl)benzoate

In a manner similar to that of Example 1(g), by reacting 27.12 g (0.10 mol) of ethyl 4-(3-bromopropyl)benzoate, 22.84 g (0.20 mol) of potassium thioacetate and 163 mg (0.6% by mass) of sodium iodide in 550 ml of methyl ethyl ketone, 21.23 g (80%) of the expected compound are collected in the form of a brown oil.

### h) Preparation of ethyl 4-(3-mercaptopropyl)benzoate

In a manner similar to that of Example 1(h), by reacting 21.22 g (0.080 mol) of ethyl 4-(2-acetylsulfanylpropyl)benzoate and 21.0 g (0.152 mol) of potassium carbonate in 220 ml of ethanol, 2.79 g (16%) of the expected compound are obtained in the form of an orange oil, along with 8.53 g (48%) of corresponding disulfide, isolated in the form of an orange oil.

### i) Preparation of ethyl 4-[3-(3,3-dimethoxy-2-oxo-1-propyl-2,3-dihydro-1H-indol-5-ylsulfanyl)propyl]benzoate

In a manner similar to that of Example 1(i), by reacting 1.34 g (6 mmol) of ethyl 4-(3-mercaptopropyl)benzoate in 1 ml of tetrahydrofuran, 4.80 g (12 mmol) of polymer-supported borohydride resin Amberlite® IRA400 (2.5 mmol/g) (Aldrich: 32864-2), 55 mg of bis(bipyridine)nickel (II) bromide (Organometallics 1985, 4, 657-661) and 1.44 g (4 mmol) of 5-iodo-3,3-dimethoxy-1-propyl-1,3-dihydroindol-2-one in 40 ml of ethanol, 1.06 g (58%) of the expected derivative are obtained in the form of an orange oil.

### j) Preparation of ethyl 4-[3-(2,3-dioxo-1-propyl-2,3-dihydro-1H-indol-5-ylsulfanyl)propyl]benzoate

In a manner similar to that of Example 1(j), by reacting 16.5 ml (33 mmol) of 2N hydrochloric acid and 1.06 g (2.3 mmol) of ethyl 4-[2-(3,3-dimethoxy-2-oxo-1-propyl-2,3-dihydro-1H-indol-5-ylsulfanyl)propyl]benzoate in 30 ml of acetone, 620 mg (66%) of the expected derivative are obtained in the form of an orange-red powder.

### k) Preparation of 4-[3-(2,3-dioxo-1-propyl-2,3-dihydro-1H-indol-5-ylsulfanyl)propyl]benzoic acid

In a manner similar to that of Example 1(k), by reacting 300 mg (0.73 mmol) of ethyl 4-[3-(2,3-dioxo-1-propyl-2,3-dihydro-1H-indol-5-ylsulfanyl)propyl]benzoate in 10 ml of methanol and 5.5 ml (11 mmol) of a 2 M solution of potassium carbonate in water, 209 mg (75%) of the expected derivative are obtained in the form of a Bordeaux-red solid. (m.p.=140-142° C.)

### l) Synthesis of 4-[3-(3-((3-trifluoromethylanilinocarbonyl)hydrazono)-2-oxo-1-propyl-2,3-dihydro-1H-indol-5-ylsulfanyl)propyl]benzoic acid

This step was performed by parallel chemistry in a 96-well plate.

In a manner similar to that of Example 3(1), by reacting 0.5 ml (0.040 mmol) of a solution of 71 mg of 4-[3-(trifluoromethyl)phenyl]semicarbazide hydrochloride in 3.5 ml of 10% acetic acid in ethanol and 1 ml (0.036 mmol) of a solution of 166 mg of 4-[3-(2,3-dioxo-1-propyl-2,3-dihydro-1H-indol-5-ylsulfanyl)propyl]benzoic acid in 12 ml of 10% acetic acid in ethanol/tetrahydrofuran (1:1), and after filtering off the precipitate, 13.7 mg of the expected derivative are obtained in the form of a golden-yellow solid.

### EXAMPLE 41

### Synthesis of 4-[3-(3-((3-chloroanilinocarbonyl)hydrazono)-2-oxo-1-hexyl-2,3-dihydro-1H-indol-5-ylsulfanyl)propyl]benzoic acid

### a) Preparation of 5-iodo-1-hexyl-1H-indole-2,3-dione

In a manner similar to that of Example 1(a), by reacting 720 mg (0.018 mol) of 60% sodium hydride, 4.10 g (0.015 mol) of 5-iodoisatin in 40 ml of dimethylformamide and 3.50 g (0.0165 mol) of 1-iodohexane dissolved in 7 ml of dimethylformamide, 4.62 g (86%) of the expected derivative are obtained in the form of a Bordeaux-red powder.

### b) Preparation of 5-iodo-3,3-dimethoxy-1-hexyl-1,3-dihydroindol-2-one

In a manner similar to that of Example 1(b), by reacting 4.2 ml (0.077 mol) of concentrated sulfuric acid and 4.60 g (0.0129 mol) of 5-iodo-1-hexyl-1H-indole-2,3-dione in 128 ml of methanol/trimethoxymethane (1:1), 5.08 g (98%) of the expected derivative are obtained in the form of an orange oil.

Steps d), e), f), g) and h) are the same as those of Example 40.

### i) Preparation of ethyl 4-[3-(3,3-dimethoxy-2-oxo-1-hexyl-2,3-dihydro-1H-indol-5-ylsulfanyl)propyl]benzoate

In a manner similar to that of Example 1(i), by reacting 1.34 g (6 mmol) of ethyl 4-(3-mercaptopropyl)benzoate in 1 ml of tetrahydrofuran, 4.80 g (12 mmol) of polymer-supported borohydride resin Amberlite® IRA400 (2.5 mmol/g) (Aldrich: 32864-2), 55 mg of bis(bipyridine)nickel (II) bromide (Organometallics 1985,4, 657-661) and 1.61 g (4 mmol) of 5-iodo-3,3-dimethoxy-1-hexyl-1,3-dihydroindol-2-one in 40 ml of ethanol, 1.26 g (63%) of the expected derivative are obtained in the form of a yellow oil.

### j) Preparation of ethyl 4-[3-(2,3-dioxo-1-hexyl-2,3-dihydro-1H-indol-5-ylsulfanyl)propyl]benzoate

In a manner similar to that of Example 1(j), by reacting 18.5 ml (37 mmol) of 2N hydrochloric acid and 1.23 g (2.46 mmol) of ethyl 4-[2-(3,3-dimethoxy-2-oxo-1-hexyl-2,3-dihydro-1H-indol-5-ylsulfanyl)propyl]benzoate in 30 ml of acetone, 870 mg (78%) of the expected derivative are obtained in the form of a blood-red powder.

### k) Preparation of 4-[3-(2,3-dioxo-1-hexyl-2,3-dihydro-1H-indol-5-ylsulfanyl)propyl]benzoic acid

In a manner similar to that of Example 1(k), by reacting 450 mg (1.0 mmol) of ethyl 4-[3-(2,3-dioxo-1-hexyl-2,3-dihydro-1H-indol-5-ylsulfanyl)propyl]benzoate in 15 ml of methanol and 7.5 ml (15 mmol) of a 2 M solution of potassium carbonate in water, 294 mg (69%) of the expected derivative are obtained in the form of a prune-colored solid. (m.p.=150-152° C.)

### l) Synthesis of 4-[3-(3-((3-chloroanilinocarbonyl)hydrazono)-2-oxo-1-hexyl-2,3-dihydro-1H-indol-5-ylsulfanyl)propyl]benzoic acid

This step was performed by parallel chemistry in a 96-well plate.

In a manner similar to that of Example 3(l), by reacting 0.5 ml (0.040 mmol) of a solution of 62 mg of 4-(3-chlorophenyl)semicarbazide hydrochloride in 3.5 ml of 10% acetic acid in ethanol and 1 ml (0.036 mmol) of a solution of 184 mg of 4-[3-(2,3-dioxo-1-hexyl-2,3-dihydro-1H-indol-5-ylsulfanyl)propyl]benzoic acid in 12 ml of 10% acetic acid in ethanol/tetrahydrofuran (1:1), and after filtering off the precipitate, 13.8 mg of the expected derivative are obtained in the form of an orange solid.

### EXAMPLE 42

### Synthesis of 4-[3-(3-((2-chloroanilinocarbonyl)hydrazono)-2-oxo-1-propyl-2,3-dihydro-1H-indol-5-ylsulfanyl)propyl]benzoic acid

Steps a), b), d), e), f), g), h), i), j) and k) are the same as those of Example 40.

### l) Synthesis of 4-[3-(3-((2-chloroanilinocarbonyl)hydrazono)-2-oxo-1-propyl-2,3-dihydro-1H-indol-5-ylsulfanyl)propyl]benzoic acid

This step was performed by parallel chemistry in a 96-well plate.

In a manner similar to that of Example 3(l), by reacting 0.5 ml (0.040 mmol) of a solution of 62 mg of 4-(2-chlorophenyl)semicarbazide hydrochloride in 3.5 ml of 10% acetic acid in ethanol and 1 ml (0.036 mmol) of a solution of 166 mg of 4-[3-(2,3-dioxo-1-propyl-2,3-dihydro-1H-indol-5-ylsulfanyl)propyl]benzoic acid in 12 ml of 10% acetic acid in ethanol/tetrahydrofuran (1:1), and after filtering off the precipitate, 12.8 mg of the expected derivative are obtained in the form of an orange solid.

### EXAMPLE 43

### Synthesis of 4-[3-(3-((4-fluoroanilinocarbonyl)hydrazono)-2-oxo-1-propyl-2,3-dihydro-1H-indol-5-ylsulfanyl)propyl]benzoic acid

Steps a), b), d), e), f), g), h), i), j) and k) are the same as those of Example 40.

### l) Synthesis of 4-[3-(3-((4-fluoroanilinocarbonyl)hydrazono)-2-oxo-1-propyl-2,3-dihydro-1H-indol-5-ylsulfanyl)propyl]benzoic acid

This step was performed by parallel chemistry in a 96-well plate.

In a manner similar to that of Example 3(l), by reacting 0.5 ml (0.040 mmol) of a solution of 57 mg of 4-(4-fluorophenyl)semicarbazide hydrochloride in 3.5 ml of 10% acetic acid in ethanol and 1 ml (0.036 mmol) of a solution of 166 mg of 4-[3-(2,3-dioxo-1-propyl-2,3-dihydro-1H-indol-5-ylsulfanyl)propyl]benzoic acid in 12 ml of 10% acetic acid in ethanol/tetrahydrofuran (1:1), and after filtering off the precipitate, 12.3 mg of the expected derivative are obtained in the form of a golden-yellow solid.

### EXAMPLE 44

### Synthesis of 4-{2-[3-((3-trifluoromethylanilinocarbonyl)hydrazono)-2-oxo-1-propyl-2,3-dihydro-1H-indol-5-ylsulfanyl]ethyl}benzoic acid

Steps a) and b) are the same as those of Example 40.

Steps c), g), h) are the same as those of Example 1.

### i) Preparation of ethyl 4-[3-(3,3-dimethoxy-2-oxo-1-propyl-2,3-dihydro-1H-indol-5-ylsulfanyl)ethyl]benzoate

In a manner similar to that of Example 1(i), by reacting 1.41 g (6.7 mmol) of ethyl 4-(3-mercaptoethyl)benzoate in 0.7 ml of tetrahydrofuran, 5.30 g (13.2 mmol) of polymer-supported borohydride resin Amberlite® IRA400 (2.5 mmol/g) (Aldrich: 32864-2), 55 mg of bis(bipyridine)nickel (II) bromide (Organometallics 1985, 4, 657-661) and 1.59 g (4.4 mmol) of 5-iodo-3,3-dimethoxy-1-propyl-1,3-dihydroindol-2-one in 45 ml of ethanol, 1.42 g (52%) of the expected derivative are obtained in the form of a yellow oil.

### j) Preparation of ethyl 4-[3-(2,3-dioxo-1-propyl-2,3-dihydro-1H-indol-5-ylsulfanyl)ethyl]benzoate

In a manner similar to that of Example 1(j), by reacting 19.5 ml (39 mmol) of 2N hydrochloric acid and 1.38 g (2.24 mmol) of ethyl 4-[2-(3,3-dimethoxy-2-oxo-1-propyl-2,3-dihydro-1H-indol-5-ylsulfanyl)ethyl]benzoate in 40 ml of acetone, 900 mg (73%) of the expected derivative are obtained in the form of a Bordeaux-red powder.

### k) Preparation of 4-[3-(2,3-dioxo-1-propyl-2,3-dihydro-1H-indol-5-ylsulfanyl)ethyl]benzoic acid

In a manner similar to that of Example 1(k), by reacting 477 mg (1.0 mmol) of ethyl 4-[3-(2,3-dioxo-1-propyl-2,3-dihydro-1H-indol-5-ylsulfanyl)ethyl]benzoate in 20 ml of methanol and 9 ml (18 mmol) of a 2 M solution of potassium carbonate in water, 346 mg (78%) of the expected derivative are obtained in the form of an orange powder. (m.p.=192-193° C.)

### l) Synthesis of 4-{2-[3-((3-trifluoromethylanilinocarbonyl)hydrazono)-2-oxo-1-propyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 71 mg of 4-[3-(trifluoromethyl)phenyl]semicarbazide hydrochloride in 3.5 ml of 10% acetic acid in ethanol and 1 ml (0.036 mmol) of a solution of 160 mg of 4-[3-(2,3-dioxo-1-propyl-2,3-dihydro-1H-indol-5-ylsulfanyl)ethyl]benzoic acid in 12 ml of 10% acetic acid in ethanol/tetrahydrofuran (1:1), and after filtering off the precipitate, 12.7 mg of the expected derivative are obtained in the form of a yellow solid.

### EXAMPLE 45

### Synthesis of 4-{2-[3-((2-chloro-3-trifluoromethylanilinocarbonyl)hydrazono)-2-oxo-1-propyl-2,3-dihydro-1H-indol-5-ylsulfanyl]ethyl}benzoic acid

Steps a), b), c), g), h), i), j) and k) are the same as those of Example 44.

### l) Synthesis of 4-{2-[3-((2-chloro-3-trifluoromethylanilinocarbonyl)hydrazono)-2-oxo-1-propyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 70 mg of N1-[2-chloro-4-(trifluoromethyl)phenyl]hydrazine-1-carboxamide in 3.5 ml of 10% acetic acid in ethanol and 1 ml (0.036 mmol) of a solution of 160 mg of 4-[3-(2,3-dioxo-1-propyl-2,3-dihydro-1H-indol-5-ylsulfanyl)ethyl]benzoic acid in 12 ml of 10% acetic acid in ethanol/tetrahydrofuran (1:1), and after filtering off the precipitate, 13.1 mg of the expected derivative are obtained in the form of a golden-yellow solid.

### EXAMPLE 46

### Synthesis of 4-[3-(3-((3-chloroanilinocarbonyl)hydrazono)-2-oxo-1-pentyl-2,3-dihydro-1H-indol-5-ylsulfanyl)propyl]benzoic acid

Steps a) and b) are the same as those of Example 1.

Steps d), e), f), g) and h) are the same as those of Example 40.

### i) Preparation of ethyl 4-[3-(3,3-dimethoxy-2-oxo-1-pentyl-2,3-dihydro-1H-indol-5-ylsulfanyl)propyl]benzoate

In a manner similar to that of Example 1(i), by reacting 1.89 g (4.24 mmol) of ethyl 4-(3-mercaptopropyl)benzoate in disulfide form in 1 ml of tetrahydrofuran, 6.78 g (16.95 mmol) of polymer-supported borohydride resin Amberlite® IRA400 (2.5 mmol/g) (Aldrich: 32864-2), 70 mg of bis(bipyridine)nickel (II) bromide (Organometallics 1985, 4, 657-661) and 2.20 g (5.65 mmol) of 5-iodo-3,3-dimethoxy-1-pentyl-1,3-dihydroindol-2-one in 60 ml of ethanol, 1.98 g (72%) of the expected derivative are obtained in the form of a yellow oil.

### j) Preparation of ethyl 4-[3-(2,3-dioxo-1-pentyl-2,3-dihydro-1H-indol-5-ylsulfanyl)propyl]benzoate

In a manner similar to that of Example 1(j), by reacting 25 ml (50 mmol) of 2N hydrochloric acid and 1.97 g (4.0 mmol) of ethyl 4-[2-(3,3-dimethoxy-2-oxo-1-pentyl-2,3-dihydro-1H-indol-5-ylsulfanyl)propyl]benzoate in 50 ml of acetone, 1.27 g (72%) of the expected derivative are obtained in the form of a blood-red powder.

### k) Preparation of 4-[3-(2,3-dioxo-1-pentyl-2,3-dihydro-1H-indol-5-ylsulfanyl)propyl]benzoic acid

In a manner similar to that of Example 1(k), by reacting 660 mg (1.5 mmol) of ethyl 4-[3-(2,3-dioxo-1-pentyl-2,3-dihydro-1H-indol-5-ylsulfanyl)propyl]benzoate in 20 ml of methanol and 11.5 ml (23 mmol) of a 2 M solution of potassium carbonate in water, 360 mg (58%) of the expected derivative are obtained in the form of a brown-red powder. (m.p.=145-146° C.)

### l) Synthesis of 4-{2-[3-((3-trifluoromethylanilinocarbonyl)hydrazono)-2-oxo-1-propyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 62 mg of 4-(3-chlorophenyl)semicarbazide hydrochloride in 3.5 ml of 10% acetic acid in ethanol and 1 ml (0.036 mmol) of a solution of 178 mg of 4-[3-(2,3-dioxo-1-pentyl-2,3-dihydro-1H-indol-5-ylsulfanyl)propyl]benzoic acid in 12 ml of 10% acetic acid in ethanol/tetrahydrofuran (1:1), and after filtering off the precipitate, 12.4 mg of the expected derivative are obtained in the form of an orange solid.

### EXAMPLE 47

### Synthesis of 4-{2-[3((aminocarbonyl)hydrazono)-2-oxo-1-propyl-2,3-dihydro-1H-indol-5-ylsulfanyl]ethyl}benzoic acid

Steps a), b), c), g), h), i), j) and k) are the same as those of Example 44.

### l) Synthesis of 4-{2-[3((aminocarbonyl)hydrazono)-2-oxo-1-propyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 31 mg of semicarbazide hydrochloride in 3.5 ml of 10% acetic acid in ethanol and 1 ml (0.036 mmol) of a solution of 160 mg of 4-[3-(2,3-dioxo-1-propyl-2,3-dihydro-1H-indol-5-ylsulfanyl)ethyl]benzoic acid in 12 ml of 10% acetic acid in ethanol/tetrahydrofuran (1:1), and after filtering off the precipitate, 8.5 mg of the expected derivative are obtained in the form of a fluorescent yellow solid.

### EXAMPLE 48

### Synthesis of 4-{3-[3((aminocarbonyl)hydrazono)-2-oxo-1-propyl-2,3-dihydro-1H-indol-5-ylsulfanyl]propyl}benzoic acid

Steps a), b), d), e), f), g), h), i), j) and k) are the same as those of Example 40.

### l) Synthesis of 4-{3-[3((aminocarbonyl)hydrazono)-2-oxo-1-propyl-2,3-dihydro-1H-indol-5-ylsulfanyl]-propyl}benzoic acid

This step was performed by parallel chemistry in a 96-well plate.

In a manner similar to that of Example 3(l), by reacting 0.5 ml (0.040 mmol) of a solution of 31 mg of semicarbazide hydrochloride in 3.5 ml of 10% acetic acid in ethanol and 1 ml (0.036 mmol) of a solution of 166 mg of 4-[3-(2,3-dioxo-1-propyl-2,3-dihydro-1H-indol-5-ylsulfanyl)propyl]benzoic acid in 12 ml of 10% acetic acid in ethanol/tetrahydrofuran (1:1), and after filtering off the precipitate, 8.7 mg of the expected derivative are obtained in the form of an orange solid.

### EXAMPLE 49

### Synthesis of 4-[3-(3-((2-chloro-4-trifluoroanilinocarbonyl)hydrazono)-2-oxo-1-propyl-2,3-dihydro-1H-indol-5-ylsulfanyl)propyl]benzoic acid

Steps a), b), d), e), f), g), h), i), j) and k) are the same as those of Example 40.

### l) Synthesis of 4-[3-(3-((2-chloro-4-trifluoroanilinocarbonyl)hydrazono)-2-oxo-1-propyl-2,3-dihydro-1H-indol-5-ylsulfanyl)propyl]benzoic acid

This step was performed by parallel chemistry in a 96-well plate.

In a manner similar to that of Example 3(l), by reacting 0.5 ml (0.040 mmol) of a solution of 70 mg of N1-[2-chloro-4-(trifluoromethyl)phenyl]hydrazine-1-carboxamide in 3.5 ml of 10% acetic acid in ethanol and 1 ml (0.036 mmol) of a solution of 166 mg of 4-[3-(2,3-dioxo-1-propyl-2,3-dihydro-1H-indol-5-ylsulfanyl)propyl]benzoic acid in 12 ml of 10% acetic acid in ethanol/tetrahydrofuran (1:1), and after filtering off the precipitate, 12 mg of the expected derivative are obtained in the form of an orange solid.

### EXAMPLE 50

### Synthesis of 4-{3-[3((aminocarbonyl)hydrazono)-2-oxo-1-hexyl-2,3-dihydro-1H-indol-5-ylsulfanyl]propyl}benzoic acid

Steps a), b), d), e), f), g), h), i), j) and k) are the same as those of Example 41.

### l) Synthesis of 4-{3-[3((aminocarbonyl)hydrazono)-2-oxo-1-hexyl-2,3-dihydro-1H-indol-5-ylsulfanyl]propyl}benzoic acid

This step was performed by parallel chemistry in a 96-well plate.

In a manner similar to that of Example 3(l), by reacting 0.5 ml (0.040 mmol) of a solution of 70 mg of N1-[2-chloro-4-(trifluoromethyl)phenyl]hydrazine-1-carboxamide in 3.5 ml of 10% acetic acid in ethanol and 1 ml (0.036 mmol) of a solution of 184 mg of 4-[3-(2,3-dioxo-1-hexyl-2,3-dihydro-1H-indol-5-ylsulfanyl)propyl]benzoic acid in 12 ml of 10% acetic acid in ethanol/tetrahydrofuran (1:1), and after filtering off the precipitate, 8.9 mg of the expected derivative are obtained in the form of a golden-yellow solid.

### EXAMPLE 52

### Synthesis of 4-{2-[3-((2-chloroanilinocarbonyl)hydrazono)-2-oxo-1-propyl-2,3-dihydro-1H-indol-5-ylsulfanyl]ethyl}benzoic acid

Steps a), b), c), g), h), i), j) and k) are the same as those of Example 44.

### l) Synthesis of 4-{2-[3-((2-chloroanilinocarbonyl)hydrazono)-2-oxo-1-propyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 62 mg of 4-(2-chlorophenyl)semicarbazide hydrochloride in 3.5 ml of 10% acetic acid in ethanol and 1 ml (0.036 mmol) of a solution of 160 mg of 4-[3-(2,3-dioxo-1-propyl-2,3-dihydro-1H-indol-5-ylsulfanyl)ethyl]benzoic acid in 12 ml of 10% acetic acid in ethanol/tetrahydrofuran (1:1), and after filtering off the precipitate, 9.3 mg of the expected derivative are obtained in the form of an orange solid.

### EXAMPLE 53

### Synthesis of 4-[3-(3-((3-chloroanilinocarbonyl)hydrazono)-2-oxo-1-propyl-2,3-dihydro-1H-indol-5-ylsulfanyl)propyl]benzoic acid

Steps a), b), d), e), f), g), h), i), j) and k) are the same as those of Example 40.

### l) Synthesis of 4-[3-(3-((3-chloroanilinocarbonyl)hydrazono)-2-oxo-1-propyl-2,3-dihydro-1H-indol-5-ylsulfanyl)propyl]benzoic acid

This step was performed by parallel chemistry in a 96-well plate.

In a manner similar to that of Example 3(l), by reacting 0.5 ml (0.040 mmol) of a solution of 62 mg of 4-(3-chlorophenyl)semicarbazide hydrochloride in 3.5 ml of 10% acetic acid in ethanol and 1 ml (0.036 mmol) of a solution of 166 mg of 4-[3-(2,3-dioxo-1-propyl-2,3-dihydro-1H-indol-5-ylsulfanyl)propyl]benzoic acid in 12 ml of 10% acetic acid in ethanol/tetrahydrofuran (1:1), and after filtering off the precipitate, 8.7 mg of the expected derivative are obtained in the form of an orange solid.

### EXAMPLE 54

### Synthesis of 4-{2-[3-((3-chlororanilinocarbonyl)hydrazono)-2-oxo-1-propyl-2,3-dihydro-1H-indol-5-ylsulfanyl]ethyl}benzoic acid

Steps a), b), c), g), h), i), j) and k) are the same as those of Example 44.

### l) Synthesis of 4-{2-[3-((3-chlororanilinocarbonyl)hydrazono)-2-oxo-1-propyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 62 mg of 4-(3-chlorophenyl)semicarbazide hydrochloride in 3.5 ml of 10% acetic acid in ethanol and 1 ml (0.036 mmol) of a solution of 160 mg of 4-[3-(2,3-dioxo-1-propyl-2,3-dihydro-1H-indol-5-ylsulfanyl)ethyl]benzoic acid in 12 ml of 10% acetic acid in ethanol/tetrahydrofuran (1:1), and after filtering off the precipitate, 8.3 mg of the expected derivative are obtained in the form of a yellow solid.

### EXAMPLE 55

### Synthesis of 4-{2-[3-((3,4-dichloroanilinocarbonyl)hydrazono)-2-oxo-1-pentyl-2,3-dihydro-1H-indol-5-ylsulfanyl]ethyl}benzoic acid

Steps a), b), c), g), h), i), j) and k) are the same as those of Example 1.

### l) Synthesis of 4-{2-[3-((3,4-dichloroanilinocarbonyl)hydrazono)-2-oxo-1-pentyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 71 mg of 4-(3,4-dichlorophenyl)semicarbazide hydrochloride in 3.5 ml of 10% acetic acid in ethanol and 1 ml (0.036 mmol) of a solution of 157 mg of 4-[2-(2,3-dioxo-1-pentyl-2,3-dihydro-1H-indol-5-ylsulfanyl)ethyl]benzoic acid in 11 ml of 10% acetic acid in ethanol/tetrahydrofuran (1:1), and after filtering off the precipitate, 7.6 mg of the expected derivative are obtained in the form of a golden-yellow solid.

### EXAMPLE 56

### Synthesis of 4-{2-[3-((3,4-dichloroanilinocarbonyl)hydrazono)-2-oxo-1-hexyl-2,3-dihydro-1H-indol-5-ylsulfanyl]ethyl}benzoic acid

Steps a) and b) are the same as those of Example 41.

Steps c), g), h) are the same as those of Example 1.

### i) Preparation of ethyl 4-[3-(3,3-dimethoxy-2-oxo-1-hexyl-2,3-dihydro-1H-indol-5-ylsulfanyl)ethyl]benzoate

In a manner similar to that of Example 44(i), by reacting 1.40 g (3.3 mmol) of ethyl 4-(3-mercaptoethyl)benzoate in disulfide form in 0.7 ml of tetrahydrofuran, 5.30 g (13.2 mmol) of polymer-supported borohydride resin Amberlite® IRA400 (2.5 mmol/g) (Aldrich: 32864-2), 55 mg of bis(bipyridine)nickel (II) bromide (Organometallics 1985, 4, 657-661) and 1.77 g (4.4 mmol) of 5-iodo-3,3-dimethoxy-1-hexyl-1,3-dihydroindol-2-one in 45 ml of ethanol, 1.21 g (57%) of the expected derivative are obtained in the form of a yellow oil.

### j) Preparation of ethyl 4-[3-(2,3-dioxo-1-hexyl-2,3-dihydro-1H-indol-5-ylsulfanyl)ethyl]benzoate

In a manner similar to that of Example 44(j), by reacting 18 ml (36 mmol) of 2N hydrochloric acid and 1.17 g (2.41 mmol) of ethyl 4-[2-(3,3-dimethoxy-2-oxo-1-hexyl-2,3-dihydro-1H-indol-5-ylsulfanyl)ethyl]benzoate in 30 ml of acetone, 913 mg (86%) of the expected derivative are obtained in the form of an orange-red powder.

### k) Preparation of 4-[3-(2,3-dioxo-1-hexyl-2,3-dihydro-1H-indol-5-ylsulfanyl)ethyl]benzoic acid

In a manner similar to that of Example 44(k), by reacting 483 mg (1.1 mmol) of ethyl 4-[3-(2,3-dioxo-1-hexyl-2,3-dihydro-1H-indol-5-ylsulfanyl)ethyl]benzoate in 20 ml of methanol and 8.7 ml (17 mmol) of a 2 M solution of potassium carbonate in water, 319 mg (70%) of the expected derivative are obtained in the form of a dark orange powder. (m.p.=174-175° C.)

### l) Synthesis of 4-{2-[3-((3,4-dichloroanilinocarbonyl)hydrazono)-2-oxo-1-hexyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 71 mg of 4-(3,4-dichlorophenyl)semicarbazide hydrochloride in 3.5 ml of 10% acetic acid in ethanol and 1 ml (0.036 mmol) of a solution of 178 mg of 4-[3-(2,3-dioxo-1-hexyl-2,3-dihydro-1H-indol-5-ylsulfanyl)ethyl]benzoic acid in 12 ml of 10% acetic acid in ethanol/tetrahydrofuran (1:1), and after filtering off the precipitate, 8.7 mg of the expected derivative are obtained in the form of a yellow solid.

### EXAMPLE 57

### Synthesis of 4-{3-[3((aminocarbonyl)hydrazono)-2-oxo-1-pentyl-2,3-dihydro-1H-indol-5-ylsulfanyl]propyl}benzoic acid

Steps a), b), d), e), f), g), h), i), j) and k) are the same as those of Example 46.

### l) Synthesis of 4-{3-[3((aminocarbonyl)hydrazono)-2-oxo-1-pentyl-2,3-dihydro-1H-indol-5-ylsulfanyl]-propyl}benzoic acid

This step was performed by parallel chemistry in a 96-well plate.

In a manner similar to that of Example 3(l), by reacting 0.5 ml (0.040 mmol) of a solution of 31 mg of semicarbazide hydrochloride in 3.5 ml of 10% acetic acid in ethanol and 1 ml (0.036 mmol) of a solution of 178 mg of 4-[3-(2,3-dioxo-1-pentyl-2,3-dihydro-1H-indol-5-ylsulfanyl)propyl]benzoic acid in 12 ml of 10% acetic acid in ethanol/tetrahydrofuran (1:1), and after filtering off the precipitate, 6.6 mg of the expected derivative are obtained in the form of a golden-yellow solid.

### EXAMPLE 58

### Synthesis of 4-(2-{3-[(morpholine-4-carbonyl)hydrazono]-2-oxo-1-propyl-2,3-dihydro-1H-indol-5-ylsulfanyl}ethyl)benzoic acid

Steps a), b), c), g), h), i), j) and k) are the same as those of Example 44.

### l) Synthesis of 4-(2-{3-[(morpholine-4-carbonyl)hydrazono]-2-oxo-1-propyl-2,3-dihydro-1H-indol-5-ylsulfanyl}ethyl)benzoic acid

This step was performed by parallel chemistry in a 96-well plate.

In a manner similar to that of Example 3(l), by reacting 0.5 ml (0.040 mmol) of a solution of 40 mg of morpholine-4-carbohydrazide in 3.5 ml of 10% acetic acid in ethanol and 1 ml (0.036 mmol) of a solution of 160 mg of 4-[3-(2,3-dioxo-1-propyl-2,3-dihydro-1H-indol-5-ylsulfanyl)ethyl]benzoic acid in 12 ml of 10% acetic acid in ethanol/tetrahydrofuran (1:1), and after evaporating the filtrate, 22.3 mg of the expected derivative are obtained.

### EXAMPLE 59

### Synthesis of 4-[3-(3-((2-chloro-4-trifluoroanilinocarbonyl)hydrazono)-2-oxo-1-hexyl-2,3-dihydro-1H-indol-5-ylsulfanyl)propyl]benzoic acid

Steps a), b), d), e), f), g), h), i), j) and k) are the same as those of Example 41.

### l) Synthesis of 4-[3-(3-((2-chloro-4-trifluoroanilinocarbonyl)hydrazono)-2-oxo-1-hexyl-2,3-dihydro-1H-indol-5-ylsulfanyl)propyl]benzoic acid

This step was performed by parallel chemistry in a 96-well plate.

In a manner similar to that of Example 3(l), by reacting 0.5 ml (0.040 mmol) of a solution of 70 mg of N1-[2-chloro-4-(trifluoromethyl)phenyl]hydrazine-1-carboxamide in 3.5 ml of 10% acetic acid in ethanol and 1 ml (0.036 mmol) of a solution of 184 mg of 4-[3-(2,3-dioxo-1-hexyl-2,3-dihydro-1H-indol-5-ylsulfanyl)propyl]benzoic acid in 12 ml of 10% acetic acid in ethanol/tetrahydrofuran (1:1), and after evaporating the filtrate, 24.3 mg of the expected derivative are obtained in the form of an orange solid.

### EXAMPLE 60

### Synthesis of 4-[3-(3-((3-fluoroanilinocarbonyl)hydrazono)-2-oxo-1-pentyl-2,3-dihydro-1H-indol-5-ylsulfanyl)propyl]benzoic acid

Steps a), b), d), e), f), g), h), i), j) and k) are the same as those of Example 46.

### l) Synthesis of 4-[3-(3-((3-fluoroanilinocarbonyl)hydrazono)-2-oxo-1-pentyl-2,3-dihydro-1H-indol-5-ylsulfanyl)propyl]benzoic acid

This step was performed by parallel chemistry in a 96-well plate.

In a manner similar to that of Example 3(l), by reacting 0.5 ml (0.040 mmol) of a solution of 47 mg of 4-(3-fluorophenyl)semicarbazide in 3.5 ml of 10% acetic acid in ethanol and 1 ml (0.036 mmol) of a solution of 178 mg of 4-[3-(2,3-dioxo-1-pentyl-2,3-dihydro-1H-indol-5-ylsulfanyl)propyl]benzoic acid in 12 ml of 10% acetic acid in ethanol/tetrahydrofuran (1:1), and after filtering off the precipitate, 20.4 mg of the expected derivative are obtained in the form of a golden-yellow solid.

### EXAMPLE 61

### Synthesis of 4-[3-(3-((3-fluoroanilinocarbonyl)hydrazono)-2-oxo-1-hexyl-2,3-dihydro-1H-indol-5-ylsulfanyl)propyl]benzoic acid

Steps a), b), d), e), f), g), h), i), j) and k) are the same as those of Example 41.

### l) Synthesis of 4-[3-(3-((3-fluoroanilinocarbonyl)hydrazono)-2-oxo-1-hexyl-2,3-dihydro-1H-indol-5-ylsulfanyl)propyl]benzoic acid

This step was performed by parallel chemistry in a 96-well plate.

In a manner similar to that of Example 3(l), by reacting 0.5 ml (0.040 mmol) of a solution of 47 mg of 4-(3-fluorophenyl)semicarbazide in 3.5 ml of 10% acetic acid in ethanol and 1 ml (0.036 mmol) of a solution of 184 mg of 4-[3-(2,3-dioxo-1-hexyl-2,3-dihydro-1H-indol-5-ylsulfanyl)propyl]benzoic acid in 12 ml of 10% acetic acid in ethanol/tetrahydrofuran (1:1), and after evaporating the filtrate, 20.4 mg of the expected derivative are obtained in the form of a golden-yellow solid.

### EXAMPLE 62

### Synthesis of 4-[3-(3-((anilinocarbonyl)hydrazono)-2-oxo-1-hexyl-2,3-dihydro-1H-indol-5-ylsulfanyl)propyl]benzoic acid

Steps a), b), d), e), f), g), h), i), j) and k) are the same as those of Example 41.

### l) Synthesis of 4-[3-(3-((anilinocarbonyl)hydrazono)-2-oxo-1-hexyl-2,3-dihydro-1H-indol-5-ylsulfanyl)propyl]benzoic acid

This step was performed by parallel chemistry in a 96-well plate.

In a manner similar to that of Example 3(l), by reacting 0.5 ml (0.040 mmol) of a solution of 36 mg (0.036 mmol) of 4-phenylsemicarbazide in 3 ml of 10% acetic acid in ethanol and 1 ml (0.036 mmol) of a solution of 184 mg of 4-[3-(2,3-dioxo-1-hexyl-2,3-dihydro-1H-indol-5-ylsulfanyl)propyl]benzoic acid in 12 ml of 10% acetic acid in ethanol/tetrahydrofuran (1:1), and after evaporating the filtrate, 19.6 mg of the expected derivative are obtained in the form of a golden-yellow solid.

### EXAMPLE 63

### Synthesis of 4-[3-(3-((3-fluoroanilinocarbonyl)hydrazono)-2-oxo-1-hexyl-2,3-dihydro-1H-indol-5-ylsulfanyl)ethyl]benzoic acid

Steps a), b), c), g), h), i), j) and k) are the same as those of Example 56.

### l) Synthesis of 4-[3-(3-((3-fluoroanilinocarbonyl)hydrazono)-2-oxo-1-hexyl-2,3-dihydro-1H-indol-5-ylsulfanyl)ethyl]benzoic acid

This step was performed by parallel chemistry in a 96-well plate.

In a manner similar to that of Example 3(l), by reacting 0.5 ml (0.040 mmol) of a solution of 47 mg of 4-(3-fluorophenyl)semicarbazide in 3.5 ml of 10% acetic acid in ethanol and 1 ml (0.036 mmol) of a solution of 178 mg of 4-[3-(2,3-dioxo-1-hexyl-2,3-dihydro-1H-indol-5-ylsulfanyl)ethyl]benzoic acid in 12 ml of 10% acetic acid in ethanol/tetrahydrofuran (1:1), and after filtering off the precipitate, 19.5 mg of the expected derivative are obtained in the form of a yellow solid.

### EXAMPLE 64

### Synthesis of 4-(3-{1-hexyl-3-[(morpholine-4-carbonyl)hydrazono]-2-oxo-2,3-dihydro-1H-indol-5-ylsulfanyl}propyl)benzoic acid

Steps a), b), d), e), f), g), h), i), j) and k) are the same as those of Example 41.

### l) Synthesis of 4-(3-{1-hexyl-3-[(morpholine-4-carbonyl)hydrazono]-2-oxo-2,3-dihydro-1H-indol-5-ylsulfanyl}propyl)benzoic acid

This step was performed by parallel chemistry in a 96-well plate.

In a manner similar to that of Example 3(l), by reacting 0.5 ml (0.040 mmol) of a solution of 40 mg of morpholine-4-carbohydrazide in 3.5 ml of 10% acetic acid in ethanol and 1 ml (0.036 mmol) of a solution of 184 mg of 4-[3-(2,3-dioxo-1-hexyl-2,3-dihydro-1H-indol-5-ylsulfanyl)propyl]benzoic acid in 12 ml of 10% acetic acid in ethanol/tetrahydrofuran (1:1), and after evaporating the filtrate, 18.9 mg of the expected derivative are obtained in the form of a golden-yellow oil.

### EXAMPLE 65

### Synthesis of 4-[3-(3-((anilinocarbonyl)hydrazono)-2-oxo-1-pentyl-2,3-dihydro-1H-indol-5-ylsulfanyl)propyl]benzoic acid

Steps a), b), d), e), f), g), h), i), j) and k) are the same as those of Example 46.

### l) Synthesis of 4-[3-(3-((anilinocarbonyl)hydrazono)-2-oxo-1-pentyl-2,3-dihydro-1H-indol-5-ylsulfanyl)propyl]benzoic acid

This step was performed by parallel chemistry in a 96-well plate.

In a manner similar to that of Example 3(l), by reacting 0.5 ml (0.040 mmol) of a solution of 36 mg (0.036 mmol) of 4-phenylsemicarbazide in 3.5 ml of 10% acetic acid in ethanol and 1 ml (0.036 mmol) of a solution of 178 mg of 4-[3-(2,3-dioxo-1-pentyl-2,3-dihydro-1H-indol-5-ylsulfanyl)propyl]benzoic acid in 12 ml of 10% acetic acid in ethanol/tetrahydrofuran (1:1), and after filtering off the precipitate, 18.4 mg of the expected derivative are obtained in the form of an orange solid.

### EXAMPLE 66

### Synthesis of 4-(3-{3-[(morpholine-4-carbonyl)hydrazono]-2-oxo-1-propyl-2,3-dihydro-1H-indol-5-ylsulfanyl}propyl)benzoic acid

Steps a), b), d), e), f), g), h), i), j) and k) are the same as those of Example 40.

### l) Synthesis of 4-(3-{3-[(morpholine-4-carbonyl)hydrazono]-2-oxo-1-propyl-2,3-dihydro-1H-indol-5-ylsulfanyl}propyl)benzoic acid

This step was performed by parallel chemistry in a 96-well plate.

In a manner similar to that of Example 3(l), by reacting 0.5 ml (0.040 mmol) of a solution of 40 mg of morpholine-4-carbohydrazide in 3.5 ml of 10% acetic acid in ethanol and 1 ml (0.036 mmol) of a solution of 166 mg of 4-[3-(2,3-dioxo-1-propyl-2,3-dihydro-1H-indol-5-ylsulfanyl)propyl]benzoic acid in 12 ml of 10% acetic acid in ethanol/tetrahydrofuran (1:1), and after evaporating the filtrate, 17.2 mg of the expected derivative are obtained in the form of an orange oil.

### EXAMPLE 67

### Synthesis of 4-(3-{3-[(morpholine-4-carbonyl)hydrazono]-2-oxo-1-pentyl-2,3-dihydro-1H-indol-5-ylsulfanyl}propyl)benzoic acid

Steps a), b), d), e), f), g), h), i), j) and k) are the same as those of Example 46.

### l) Synthesis of 4-(3-{3-[(morpholine-4-carbonyl)hydrazono]-2-oxo-1-pentyl-2,3-dihydro-1H-indol-5-ylsulfanyl}propyl)benzoic acid

This step was performed by parallel chemistry in a 96-well plate.

In a manner similar to that of Example 3(l), by reacting 0.5 ml (0.040 mmol) of a solution of 40 mg of morpholine-4-carbohydrazide in 3.5 ml of 10% acetic acid in ethanol and 1 ml (0.036 mmol) of a solution of 178 mg of 4-[3-(2,3-dioxo-1-pentyl-2,3-dihydro-1H-indol-5-ylsulfanyl)propyl]benzoic acid in 12 ml of 10% acetic acid in ethanol/tetrahydrofuran (1:1), and after evaporating the filtrate, 18 mg of the expected derivative are obtained in the form of an orange oil.

### EXAMPLE 68

### Synthesis of 4-{2-[3-((4-fluoroanilinocarbonyl)hydrazono)-2-oxo-1-propyl-2,3-dihydro-1H-indol-5-ylsulfanyl]ethyl}benzoic acid

Steps a), b), c), g), h), i), j) and k) are the same as those of Example 44.

### l) Synthesis of 4-{2-[3-((4-fluoroanilinocarbonyl)hydrazono)-2-oxo-1-propyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 57 mg of 4-(4-fluorophenyl)semicarbazide hydrochloride in 3.5 ml of 10% acetic acid in ethanol and 1 ml (0.036 mmol) of a solution of 160 mg of 4-[3-(2,3-dioxo-1-propyl-2,3-dihydro-1H-indol-5-ylsulfanyl)ethyl]benzoic acid in 12 ml of 10% acetic acid in ethanol/tetrahydrofuran (1:1), and after filtering off the precipitate, 16.4 mg of the expected derivative are obtained in the form of a yellow solid.

### EXAMPLE 69

### Synthesis of 4-[3-(3-((3-fluoroanilinocarbonyl)hydrazono)-2-oxo-1-propyl-2,3-dihydro-1H-indol-5-ylsulfanyl)propyl]benzoic acid

Steps a), b), d), e), f), g), h), i), j) and k) are the same as those of Example 40.

### l) Synthesis of 4-[3-(3-((3-fluoroanilinocarbonyl)hydrazono)-2-oxo-1-propyl-2,3-dihydro-1H-indol-5-ylsulfanyl)propyl]benzoic acid

This step was performed by parallel chemistry in a 96-well plate.

In a manner similar to that of Example 3(l), by reacting 0.5 ml (0.040 mmol) of a solution of 47 mg of 4-(3-fluorophenyl)semicarbazide in 3.5 ml of 10% acetic acid in ethanol and 1 ml (0.036 mmol) of a solution of 166 mg of 4-[3-(2,3-dioxo-1-propyl-2,3-dihydro-1H-indol-5-ylsulfanyl)propyl]benzoic acid in 12 ml of 10% acetic acid in ethanol/tetrahydrofuran (1:1), and after filtering off the precipitate, 16.8 mg of the expected derivative are obtained in the form of an orange solid.

### EXAMPLE 70

### Synthesis of 4-{2-[3-((anilinocarbonyl)hydrazono)-2-oxo-1-hexyl-2,3-dihydro-1H-indol-5-ylsulfanyl]ethyl}benzoic acid

Steps a), b), c), g), h), i), j) and k) are the same as those of Example 56.

### l) Synthesis of 4-{2-[3-((anilinocarbonyl)hydrazono)-2-oxo-1-hexyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 36 mg (0.036 mmol) of 4-phenylsemicarbazide in 3.5 ml of 10% acetic acid in ethanol and 1 ml (0.036 mmol) of a solution of 178 mg of 4-[3-(2,3-dioxo-1-hexyl-2,3-dihydro-1H-indol-5-ylsulfanyl)ethyl]benzoic acid in 12 ml of 10% acetic acid in ethanol/tetrahydrofuran (1:1), and after filtering off the precipitate, 16.6 mg of the expected derivative are obtained in the form of a yellow solid.

### EXAMPLE 71

### Synthesis of 4-[3-(3-((4-fluoroanilinocarbonyl)hydrazono)-2-oxo-1-pentyl-2,3-dihydro-1H-indol-5-ylsulfanyl)propyl]benzoic acid

Steps a), b), d), e), f), g), h), i), j) and k) are the same as those of Example 46.

### l) Synthesis of 4-[3-(3-((4-fluoroanilinocarbonyl)hydrazono)-2-oxo-1-pentyl-2,3-dihydro-1H-indol-5-ylsulfanyl)propyl]benzoic acid

This step was performed by parallel chemistry in a 96-well plate.

In a manner similar to that of Example 3(l), by reacting 0.5 ml (0.040 mmol) of a solution of 57 mg of 4-(4-fluorophenyl)semicarbazide hydrochloride in 3.5 ml of 10% acetic acid in ethanol and 1 ml (0.036 mmol) of a solution of 178 mg of 4-[3-(2,3-dioxo-1-pentyl-2,3-dihydro-1H-indol-5-ylsulfanyl)propyl]benzoic acid in 12 ml of 10% acetic acid in ethanol/tetrahydrofuran (1:1), and after filtering off the precipitate, 16.7 mg of the expected derivative are obtained in the form of an orange solid.

### EXAMPLE 72

### Synthesis of 4-(2-{3-[(morpholine-4-carbonyl)hydrazono]-2-oxo-1-pentyl-2,3-dihydro-1H-indol-5-ylsulfanyl}ethyl)benzoic acid

Steps a), b), c), g), h), i), j) and k) are the same as those of Example 1.

### l) Synthesis of 4-(2-{3-[(morpholine-4-carbonyl)hydrazono]-2-oxo-1-pentyl-2,3-dihydro-1H-indol-5-ylsulfanyl}ethyl)benzoic acid

This step was performed by parallel chemistry in a 96-well plate.

In a manner similar to that of Example 3(l), by reacting 0.5 ml (0.040 mmol) of a solution of 40 mg of morpholine-4-carbohydrazide in 3.5 ml of 10% acetic acid in ethanol and 1 ml (0.036 mmol) of a solution of 157 mg of 4-[2-(2,3-dioxo-1-pentyl-2,3-dihydro-1H-indol-5-ylsulfanyl)ethyl]benzoic acid in 11 ml of 10% acetic acid in ethanol/tetrahydrofuran (1:1), and after evaporating the filtrate, 15.5 mg of the expected derivative are obtained in the form of a golden-yellow solid.

### EXAMPLE 73

### Synthesis of 4-(2-{1-hexyl-3-[(morpholine-4-carbonyl)hydrazono]-2-oxo-2,3-dihydro-1H-indol-5-ylsulfanyl}ethyl)benzoic acid

Steps a), b), c), g), h), i), j) and k) are the same as those of Example 56.

### l) Synthesis of 4-(2-{1-hexyl-3-[(morpholine-4-carbonyl)hydrazono]-2-oxo-2,3-dihydro-1H-indol-5-ylsulfanyl}ethyl)benzoic acid

This step was performed by parallel chemistry in a 96-well plate.

In a manner similar to that of Example 3(l), by reacting 0.5 ml (0.040 mmol) of a solution of 40 mg of morpholine-4-carbohydrazide in 3.5 ml of 10% acetic acid in ethanol and 1 ml (0.036 mmol) of a solution of 178 mg of 4-[3-(2,3-dioxo-1-hexyl-2,3-dihydro-1H-indol-5-ylsulfanyl)ethyl]benzoic acid in 12 ml of 10% acetic acid in ethanol/tetrahydrofuran (1:1), and after evaporating the filtrate, 15.6 mg of the expected derivative are obtained in the form of a golden-yellow solid.

### EXAMPLE 74

### Synthesis of 4-[3-(3-((4-fluoroanilinocarbonyl)hydrazono)-2-oxo-1-hexyl-2,3-dihydro-1H-indol-5-ylsulfanyl)propyl]benzoic acid

Steps a), b), d), e), f), g), h), i), j) and k) are the same as those of Example 41.

### l) Synthesis of 4-[3-(3-((4-fluoroanilinocarbonyl)hydrazono)-2-oxo-1-hexyl-2,3-dihydro-1H-indol-5-ylsulfanyl)propyl]benzoic acid

This step was performed by parallel chemistry in a 96-well plate.

In a manner similar to that of Example 3(l), by reacting 0.5 ml (0.040 mmol) of a solution of 57 mg of 4-(4-fluorophenyl)semicarbazide hydrochloride in 3.5 ml of 10% acetic acid in ethanol and 1 ml (0.036 mmol) of a solution of 184 mg of 4-[3-(2,3-dioxo-1-hexyl-2,3-dihydro-1H-indol-5-ylsulfanyl)propyl]benzoic acid in 12 ml of 10% acetic acid in ethanol/tetrahydrofuran (1:1), and after filtering off the precipitate, 16.7 mg of the expected derivative are obtained in the form of a golden-yellow solid.

### EXAMPLE 75

### Synthesis of 4-[3-(3-((3-trifluoromethylanilinocarbonyl)hydrazono)-2-oxo-1-pentyl-2,3-dihydro-1H-indol-5-ylsulfanyl)propyl]benzoic acid

Steps a), b), d), e), f), g), h), i), j) and k) are the same as those of Example 46.

### l) Synthesis of 4-[3-(3-((3-trifluoromethylanilinocarbonyl)hydrazono)-2-oxo-1-pentyl-2,3-dihydro-1H-indol-5-ylsulfanyl)propyl]benzoic acid

This step was performed by parallel chemistry in a 96-well plate.

In a manner similar to that of Example 3(l), by reacting 0.5 ml (0.040 mmol) of a solution of 71 mg of 4-[3-(trifluoromethyl)phenyl]semicarbazide hydrochloride in 3.5 ml of 10% acetic acid in ethanol and 1 ml (0.036 mmol) of a solution of 178 mg of 4-[3-(2,3-dioxo-1-pentyl-2,3-dihydro-1H-indol-5-ylsulfanyl)propyl]benzoic acid in 12 ml of 10% acetic acid in ethanol/tetrahydrofuran (1:1), and after filtering off the precipitate, 17.9 mg of the expected derivative are obtained in the form of an orange solid.

### EXAMPLE 76

### Synthesis of 4-[3-(3-((3-trifluoromethylanilinocarbonyl)hydrazono)-2-oxo-1-hexyl-2,3-dihydro-1H-indol-5-ylsulfanyl)propyl]benzoic acid

Steps a), b), d), e), f), g), h), i), j) and k) are the same as those of Example 41.

### l) Synthesis of 4-[3-(3-((3-trifluoromethylanilinocarbonyl)hydrazono)-2-oxo-1-hexyl-2,3-dihydro-1H-indol-5-ylsulfanyl)propyl]benzoic acid

This step was performed by parallel chemistry in a 96-well plate.

In a manner similar to that of Example 3(l), by reacting 0.5 ml (0.040 mmol) of a solution of 71 mg of 4-[3-(trifluoromethyl)phenyl]semicarbazide hydrochloride in 3.5 ml of 10% acetic acid in ethanol and 1 ml (0.036 mmol) of a solution of 184 mg of 4-[3-(2,3-dioxo-1-hexyl-2,3-dihydro-1H-indol-5-ylsulfanyl)propyl]benzoic acid in 12 ml of 10% acetic acid in ethanol/tetrahydrofuran (1:1), and after filtering off the precipitate, 17.9 mg of the expected derivative are obtained in the form of an orange solid.

### EXAMPLE 77

### Synthesis of 4-{2-[3-((3-fluoroanilinocarbonyl)hydrazono)-2-oxo-1-pentyl-2,3-dihydro-1H-indol-5-ylsulfanyl]ethyl)benzoic acid

Steps a), b), c), g), h), i), j) and k) are the same as those of Example 1.

### l) 4 Synthesis of -{2-[3-((3-fluoroanilinocarbonyl)hydrazono)-2-oxo-1-pentyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 47 mg of 4-(3-fluorophenyl)semicarbazide in 3.5 ml of 10% acetic acid in ethanol and 1 ml (0.036 mmol) of a solution of 157 mg of 4-[2-(2,3-dioxo-1-pentyl-2,3-dihydro-1H-indol-5-ylsulfanyl)ethyl]benzoic acid in 11 ml of 10% acetic acid in ethanol/tetrahydrofuran (1:1), and after filtering off the precipitate, 15.6 mg of the expected derivative are obtained in the form of a yellow solid.

### EXAMPLE 78

### Synthesis of 4-{2-[3-((3,4-dichloroanilinocarbonyl)hydrazono)-2-oxo-1-propyl-2,3-dihydro-1H-indol-5-ylsulfanyl]ethyl}benzoic acid

Steps a), b), c), g), h), i), j) and k) are the same as those of Example 44.

### l) Synthesis of 4-{2-[3-((3,4-dichloroanilinocarbonyl)hydrazono)-2-oxo-1-propyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 71 mg of 4-(3,4-dichlorophenyl)semicarbazide hydrochloride in 3.5 ml of 10% acetic acid in ethanol and 1 ml (0.036 mmol) of a solution of 160 mg of 4-[3-(2,3-dioxo-1-propyl-2,3-dihydro-1H-indol-5-ylsulfanyl)ethyl]benzoic acid in 12 ml of 10% acetic acid in ethanol/tetrahydrofuran (1:1), and after filtering off the precipitate, 16 mg of the expected derivative are obtained in the form of a golden-yellow solid.

### EXAMPLE 79

### Synthesis of 4-{2-[3-((anilinocarbonyl)hydrazono)-2-oxo-1-propyl-2,3-dihydro-1H-indol-5-ylsulfanyl]ethyl}benzoic acid

Steps a), b), c), g), h), i), j) and k) are the same as those of Example 44.

### l) Synthesis of 4-{2-[3-((anilinocarbonyl)hydrazono)-2-oxo-1-propyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 36 mg (0.036 mmol) of 4-phenylsemicarbazide in 3.5 ml of 10% acetic acid in ethanol and 1 ml (0.036 mmol) of a solution of 160 mg of 4-[3-(2,3-dioxo-1-propyl-2,3-dihydro-1H-indol-5-ylsulfanyl)ethyl]benzoic acid in 12 ml of 10% acetic acid in ethanol/tetrahydrofuran (1:1), and after filtering off the precipitate, 14 mg of the expected derivative are obtained in the form of a golden-yellow solid.

### EXAMPLE 81

### Synthesis of 4-{2-[3-((3-trifluoromethylanilino)carbonylhydrazono)-2-oxo-1-hexyl-2,3-dihydro-1H-indol-5-ylsulfanyl]ethyl}benzoic acid

Steps a), b), c), g), h), i), j) and k) are the same as those of Example 56.

### l) Synthesis of 4-{2-[3-((3-trifluoromethylanilino)carbonylhydrazono)-2-oxo-1-hexyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 71 mg of 4-[3-(trifluoromethyl)phenyl]semicarbazide hydrochloride in 3.5 ml of 10% acetic acid in ethanol and 1 ml (0.036 mmol) of a solution of 178 mg of 4-[3-(2,3-dioxo-1-hexyl-2,3-dihydro-1H-indol-5-ylsulfanyl)ethyl]benzoic acid in 12 ml of 10% acetic acid in ethanol/tetrahydrofuran (1:1), and after filtering off the precipitate, 16.2 mg of the expected derivative are obtained in the form of a yellow solid.

### EXAMPLE 82

### Synthesis of 4-{2-[3-((4-fluoroanilino)carbonylhydrazono)-2-oxo-1-hexyl-2,3-dihydro-1H-indol-5-ylsulfanyl]ethyl}benzoic acid

Steps a), b), c), g), h), i), j) and k) are the same as those of Example 56.

### l) Synthesis of 4-{2-[3-((4-fluoroanilino)carbonylhydrazono)-2-oxo-1-hexyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 57 mg of 4-(4-fluorophenyl)semicarbazide hydrochloride in 3.5 ml of 10% acetic acid in ethanol and 1 ml (0.036 mmol) of a solution of 178 mg of 4-[3-(2,3-dioxo-1-hexyl-2,3-dihydro-1H-indol-5-ylsulfanyl)ethyl]benzoic acid in 12 ml of 10% acetic acid in ethanol/tetrahydrofuran (1:1), and after filtering off the precipitate, 14.2 mg of the expected derivative are obtained in the form of a yellow solid.

### EXAMPLE 83

### Synthesis of ethyl 4-{2-[3-((2-chloroanilinocarbonyl)hydrazono)-2-oxo-1-heptyl-2,3-dihydro-1H-indol-5-ylsulfanyl]ethyl}benzoate

### a) Preparation of 5-iodo-1-heptyl-1H-indole-2,3-dione

1.6 g (0.04 mol) of 60% sodium hydride are added portionwise to a mixture of 10 g (0.036 mol) of 5-iodoisatin in 100 ml of dimethylformamide. The reaction medium is stirred at room temperature for 30 minutes. 6.3 ml (0.04 mol) of 1-bromoheptane dissolved in 20 ml of dimethylformamide are then added dropwise. The reaction medium is stirred at room temperature overnight. The reaction medium is then poured into saturated ammonium chloride solution and extracted with ethyl acetate. The organic phases are combined, washed with water, dried over magnesium sulfate and concentrated on a rotary evaporator under vacuum. The solid is washed with heptane and dried. 11.8 g (87%) of the expected compound are collected in the form of an orange powder.

### b) Preparation of 5-iodo-3,3-dimethoxy-1-heptyl-1,3-dihydroindol-2-one

10 ml of concentrated sulfuric acid are added dropwise to a mixture of 11.8 g (31.8 mmol) of 5-iodo-1-heptyl-1H-indole-2,3-dione in 160 ml of methanol/trimethoxymethane (1:1). The reaction medium is then stirred at room temperature overnight. 3.4 ml of concentrated sulfuric acid are added dropwise to the mixture. The reaction medium is stirred for 5 hours 30 minutes at room temperature and then poured into ice-water solution and neutralized to pH 8 with sodium hydrogen carbonate. The desired product is extracted with ethyl ether. The organic phases are combined, washed with water, dried over magnesium sulfate and concentrated on a rotary evaporator under vacuum. After evaporating off the solvent, the expected compound (13.8 g; 100%) is isolated in the form of an orange-brown oil.

Steps c), g), h) are the same as those of Example 1.

### i) Preparation of ethyl 4-[2-(3,3-dimethoxy-2-oxo-1-heptyl-2,3-dihydro-1H-indol-5-ylsulfanyl)ethyl]benzoate

In a manner similar to that of Example 1(i), by reacting 7.8 g (37 mmol) of ethyl 4-(2-mercaptoethyl)benzoate in 35 ml of tetrahydrofuran, 30 g (74 mmol) of polymer-supported borohydride resin Amberlite® IRA400 (2.5 mmol/g) (Aldrich: 32864-2), 320 mg of bis(bipyridine)nickel (II) bromide (Organometallics 1985, 4, 657-661) and 10.3 mg (24.7 mmol) of 5-iodo-3,3-dimethoxy-1-heptyl-1,3-dihydroindol-2-one in 200 ml of ethanol, 11.2 g (90%) of the expected derivative are obtained in the form of a yellow oil.

### j) Preparation of ethyl 4-[2-(2,3-dioxo-1-heptyl-2,3-dihydro-1H-indol-5-ylsulfanyl)ethyl]benzoate

In a manner similar to that of Example 1(j), by reacting 112 ml of 1 N hydrochloric acid, 126 ml of 2N hydrochloric acid and 11.2 g (22.4 mmol) of ethyl 4-[2-(3,3-dimethoxy-2-oxo-1-heptyl-2,3-dihydro-1H-indol-5-ylsulfanyl)ethyl]benzoate in 330 ml of acetone, 9 g (88%) of the expected derivative are obtained in the form of a red solid.

### l) Synthesis of ethyl 4-{2-[3-((2-chloroanilinocarbonyl)hydrazono)-2-oxo-1-heptyl-2,3-dihydro-1H-indol-5-ylsulfanyl]ethyl}benzoate

This step was performed by parallel chemistry in a 96-well plate.

In a manner similar to that of Example 3(l), by reacting 0.5 ml (0.040 mmol) of a solution of 79.1 mg of 4-(2-chlorophenyl)semicarbazide hydrochloride in 4.5 ml of 10% acetic acid in methanol and 0.5 ml (0.036 mmol) of a solution of 49 mg of ethyl 4-[2-(2,3-dioxo-1-heptyl-2,3-dihydro-1H-indol-5-ylsulfanyl)ethyl]benzoate in 1.5 ml of 10% acetic acid in methanol.

### EXAMPLE 84

### Synthesis of 4-{2-[3-((2-chloroanilino)carbonylhydrazono)-2-oxo-1-heptyl-2,3-dihydro-1H-indol-5-ylsulfanyl]ethyl}benzoic acid

The starting material used is product j) obtained in Example 83.

### k) Preparation of 4-[2-(2,3-dioxo-1-heptyl-2,3-dihydro-1H-indol-5-ylsulfanyl)ethyl]benzoic acid

A mixture of 6 g (13.2 mmol) of ethyl 4-[2-(2,3-dioxo-1-heptyl-2,3-dihydro-1H-indol-5-ylsulfanyl)ethyl]benzoate in 240 ml of methanol and 100 ml of a 2 M solution of potassium carbonate in water is heated at 60-65° C. for 3 hours and then concentrated on a rotary evaporator under vacuum. Water is added to the residue obtained. The solution is acidified by addition of concentrated hydrochloric acid. The desired product is extracted with ethyl acetate and the organic phases are combined, washed with water, dried over magnesium sulfate and concentrated on a rotary evaporator under vacuum. The expected compound (4.9 g; 87%) is isolated in the form of a black powder.

### l) Synthesis of 4-{2-[3-((2-chloroanilino)carbonylhydrazono)-2-oxo-1-heptyl-2,3-dihydro-1H-indol-5-ylsulfanyl]ethyl}benzoic acid

This step was performed by parallel chemistry in a 96-well plate.

In a manner similar to that of Example 3(l), by reacting 0.5 ml (0.040 mmol) of a solution of 79.1 mg of 4-(2-chlorophenyl)semicarbazide hydrochloride in 4.5 ml of 10% acetic acid in methanol and 0.5 ml (0.036 mmol) of a solution of 107.3 mg of 4-[2-(2,3-dioxo-1-heptyl-2,3-dihydro-1H-indol-5-ylsulfanyl)ethyl]benzoic acid in 3.5 ml of 10% acetic acid in methanol.

### EXAMPLE 85

### Analyses of the Compounds Synthesized

The products were analyzed by HPLC/Mass.

Column: THERMOQUEST-HYPERSIL HyPURITY Elite C18, 150×2.1 mm, 5 μm.

Mobile phase: A (CH₃CN/0.1% HCO₂H); B (H₂O/0.1% HCO₂H), Waters Alliance 2790 LC Mobile Phase

| | | |
|---|---|---|
| Solvents | A % | 10.0 Solvent A |
| | B % | 90.0 Solvent B |

| | |
|---|---|
| Flow rate (ml/min) | 0.5 |
| Analysis time (min) | 5.00 |
| Column temperature (° C.) | 60 |
| Limit column temperature (° C.) | 10 |

Waters Alliance 2790 LC Rapid Equilibration

| | |
|---|---|
| System time (min) | 0.30 |
| Re-equilibration time (min) | 0.50 |

The gradient contains three entries, which are:

| Time | A % | B % | Flow rate | Curve |
|---|---|---|---|---|
| 0.00 | 5.0 | 65.0 | 0.450 | 1 |
| 3.00 | 95.0 | 5.0 | 0.450 | 6 |
| 5.00 | 95.0 | 5.0 | 0.450 | 6 |

| | | HPLC | |
|---|---|---|---|
| | | (% | |
| | | total | MASS |
| | | of the | ES |
| Structure | Name | area) | (M + H+) |
| 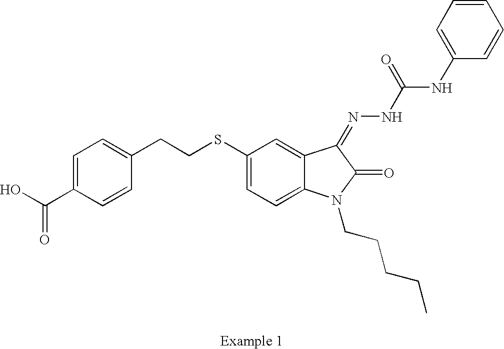 | 4-{2-[3Z-((anilinocarbonyl) hydrazono)-2-oxo-1- pentyl-2,3-dihydro-1H-indol-5- ylsulfanyl]ethyl}benzoic acid | Purity: 97.0% [12.9 min] | API-ES positive [531.2] |
| 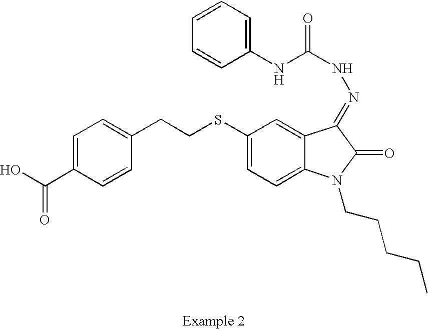 | 4-{2-[3E-((anilinocarbonyl) hydrazono)-2-oxo-1- pentyl-2,3-dihydro-1H-indol-5- ylsulfanyl]ethyl}benzoic acid | Purity: 98.0% [11.4 min] | API-ES positive [531.2] |
| 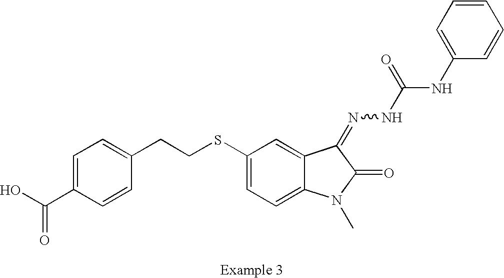 | 4-{2-[3-((anilinocarbonyl) hydrazono)-2-oxo-1- methyl-2,3-dihydro-1H-indol-5- ylsulfanyl]ethyl}benzoic acid | Purity: 71.0% | [475] +24% anti |
| 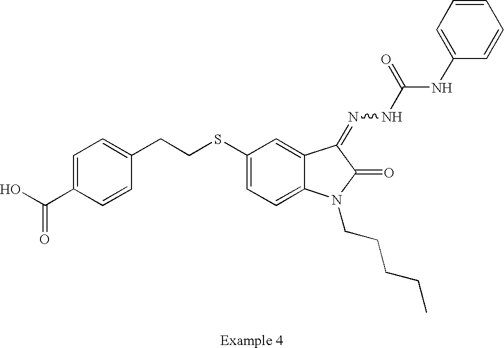 | 4-{2-[3-((anilinocarbonyl) hydrazono)-2-oxo-1- pentyl-2,3-dihydro-1H-indol-5- ylsulfanyl]ethyl}benzoic acid | Purity: 73.0% | [531] +21% anti |
| 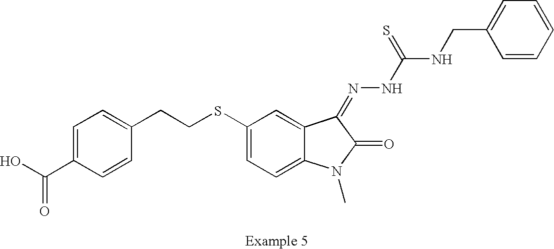 | 4-[((3Z)-3-{[(benzylamino) carbonothioyl]hydrazono}-2-oxo- 1-methyl-2,3-dihydro-1H-indol-5- ylsulfanyl]ethyl}benzoic acid | Purity: 69.0% | [505.0] |
| 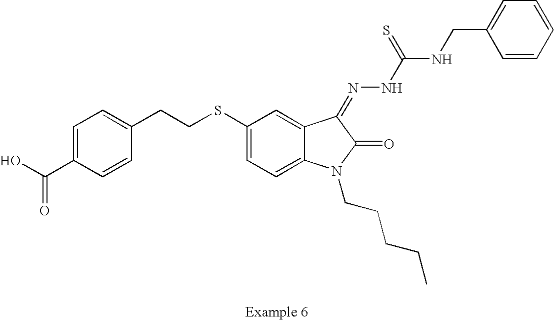 | 4-{2-[3-(benzylamino- carbonothioylhydrazono)-2- oxo-1-pentyl-2,3-dihydro-1H- indol-5-ylsulfanyl]ethyl}benzoic acid | Purity: 88.0% | [561] |
| 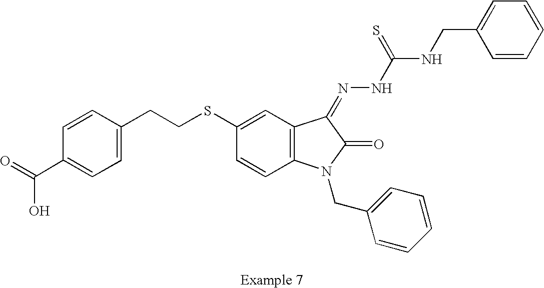 | 4-{2-[3-(benzylamino- carbonothioylhydrazono)-2- oxo-1-benzyl-2,3-dihydro-1H- indol-5-ylsulfanyl]ethyl}benzoic acid | Purity: 90.0% | [581.0] |
| 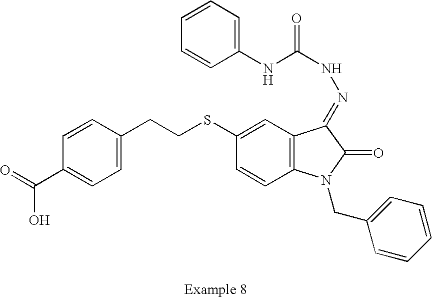 | 4-{2-[3-((anilinocarbonyl) hydrazono)-2-oxo-1- benzyl-2,3-dihydro-1H-indol-5- ylsulfanyl]ethyl}benzoic acid | Purity: 91.0% | [551] |
| 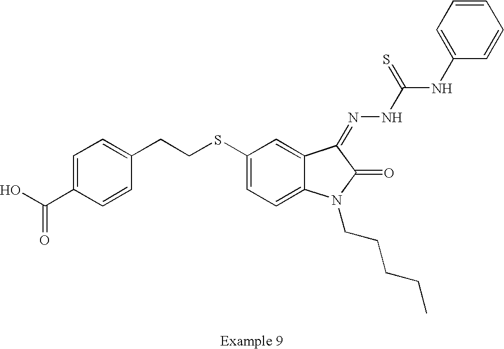 | 4-{2-[3-(anilinocarbonothioyl- hydrazono)-2-oxo-1-pentyl-2,3- dihydro-1H-indol-5-ylsulfanyl]ethyl}benzoic acid | Purity: 87.0% | [547, M + 23] |
| 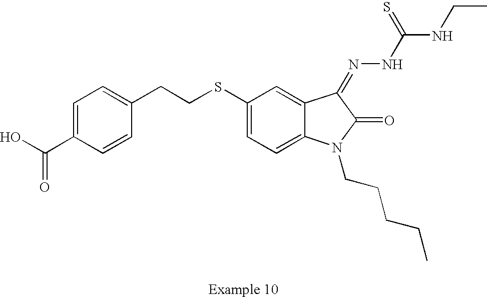 | 4-{2-[3-(ethylaminocarbonothioyl- hydrazono)-2-oxo-1-pentyl-2,3- dihydro-1H-indol-5-ylsulfanyl]ethyl}benzoic acid | Purity: 94.0% | [499] |
| 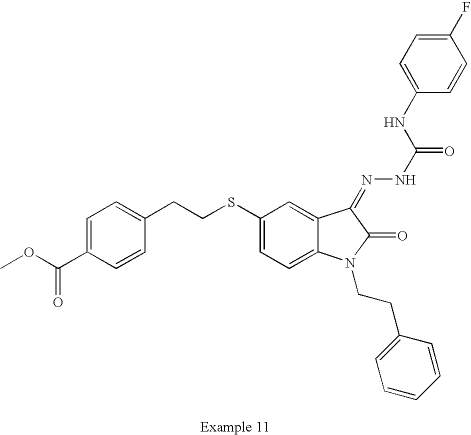 | methyl 4-{2-[3-((4-fluoroanilino) carbonylhydrazono)-2-oxo-1- phenethyl-2,3-dihydro-1H-indol-5- ylsulfanyl]ethyl}benzoate | Purity: 96.0% | [597.2] |
| 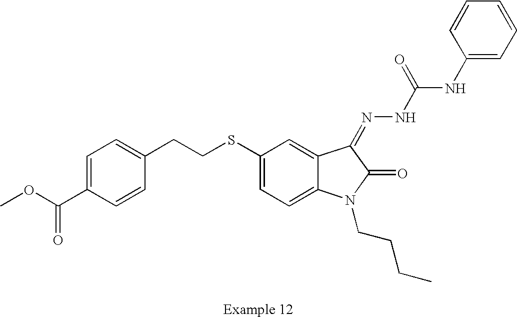 | methyl 4-{2-[3-((anilinocarbonyl) hydrazono)-2-oxo-1-butyl-2,3- dihydro-1H-indol-5- ylsulfanyl]ethyl}benzoate | Purity: 77.0% | [531] |
| 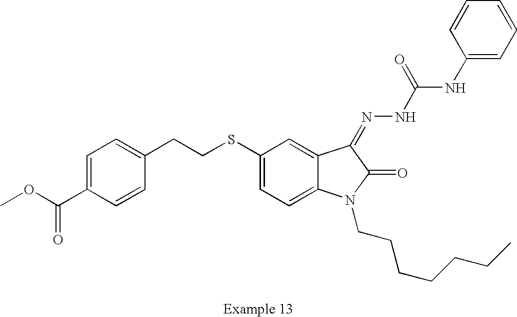 | methyl 4-{2-[3-((anilinocarbonyl) hydrazono)-2-oxo-1-heptyl-2,3- dihydro-1H-indol-5- ylsulfanyl]ethyl}benzoate | Purity: 75.0% | [573] |
| 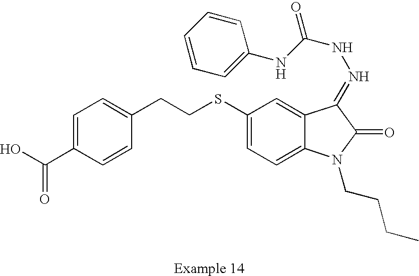 | 4-{2-[3-((anilinocarbonyl) hydrazono)-2-oxo-1- butyl-2,3-dihydro-1H-indol-5- ylsulfanyl]ethyl}benzoic acid | Purity: 90.0% | [517] |
| 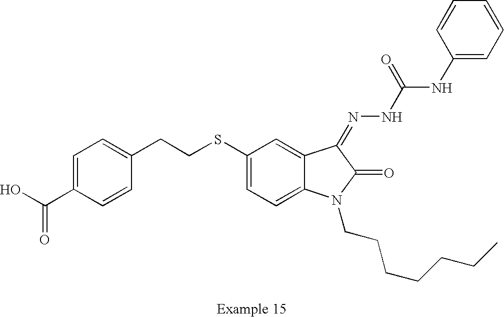 | 4-{2-[3-((anilinocarbonyl) hydrazono)-2-oxo-1- heptyl-2,3-dihydro-1H-indol-5- ylsulfanyl]ethyl}benzoic acid | Purity: 79.0% | [559] +16% ND |
| 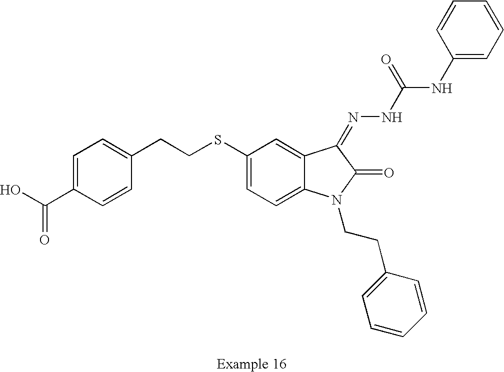 | 4-{2-[3-((anilinocarbonyl) hydrazono)-2-oxo-1- phenethyl-2,3-dihydro-1H-indol-5- ylsulfanyl]ethyl}benzoic acid | Purity: 78.0% | [565] |
| 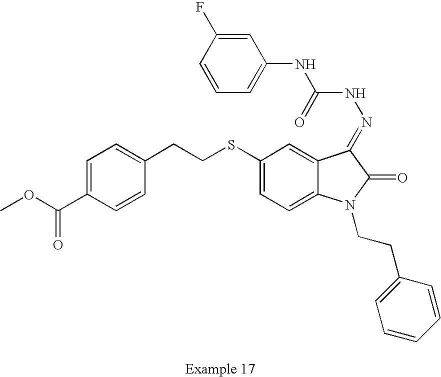 | methyl 4-{2-[3-((3-fluoroanilino- carbonyl)hydrazono)-2-oxo-1- phenethyl-2,3-dihydro-1H-indol-5- ylsulfanyl]ethyl}benzoate | Purity: 77.0% | [597] +13% syn |
| 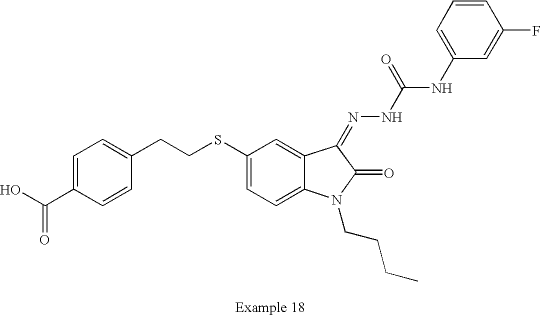 | 4-{2-[3-((3-fluoroanilino- carbonyl)hydrazono)-2-oxo-1- butyl-2,3-dihydro-1H-indol-5- ylsulfanyl]ethyl}benzoic acid | Purity: 91.0% | [535] |
| 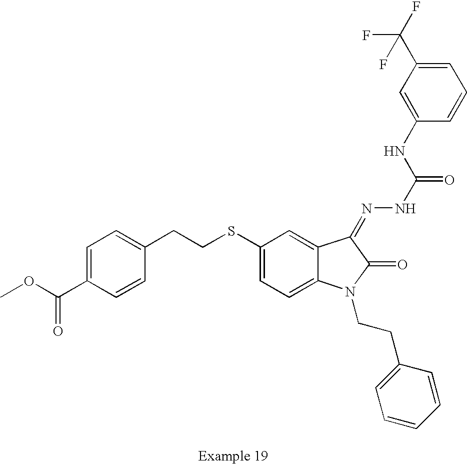 | methyl 4-{2-[3-((3-trifluoromethyl- anilinocarbonyl)hydrazono)-2-oxo- 1-phenethyl-2,3-dihydro-1H-indol- 5-ylsulfanyl]ethyl}benzoate | Purity: 93.0% | [647] |
| 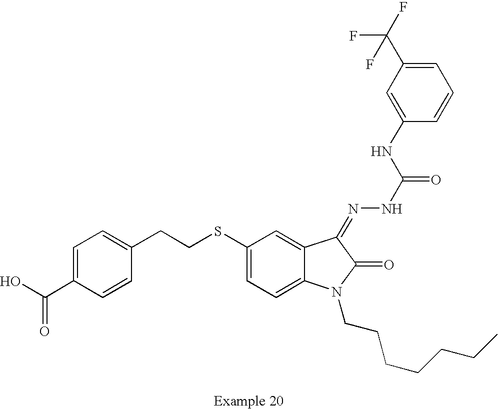 | 4-{2-[3-((3-trifluoromethyl- anilinocarbonyl)hydrazono)-2-oxo- 1-heptyl-2,3-dihydro-1H-indol-5- ylsulfanyl]ethyl}benzoic acid | Purity: 86.0% | [627] |
| 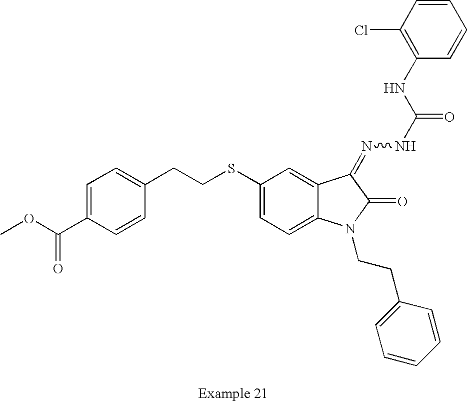 | methyl 4-{2-[3((2-chloroanilino- carbonyl)hydrazono)-2-oxo-1- phenethyl-2,3-dihydro-1H-indol-5- ylsulfanyl]ethyl}benzoate | Purity: 76.0% | [613] +17% anti |
| 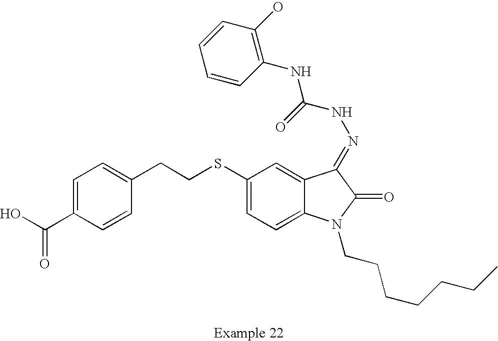 | 4-{2-[3((2-chloroanilino- carbonyl)hydrazono)-2-oxo-1- heptyl-2,3-dihydro-1H-indol-5- ylsulfanyl]ethyl}benzoic acid | Purity: 78.0% | [593] +14% “syn” |
| 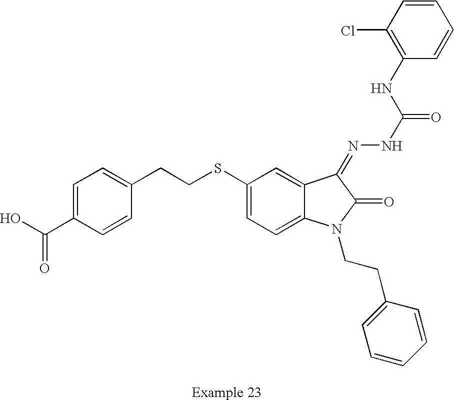 | 4-{2-[3((2-chloroanilino- carbonyl)hydrazono)-2-oxo-1- phenethyl-2,3-dihydro-1H-indol-5- ylsulfanyl]ethyl}benzoic acid | Purity: 85.0% | [599] |
| 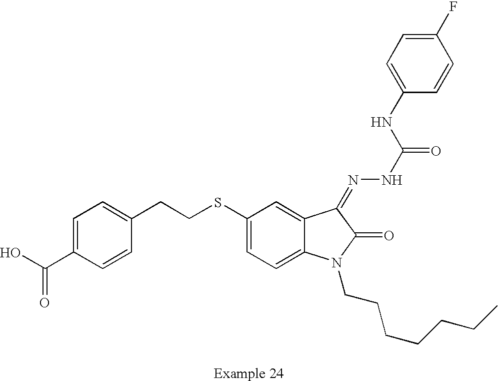 | 4-{2-[3((4-fluoroanilino- carbonyl)hydrazono)-2-oxo-1- heptyl-2,3-dihydro-1H-indol-5- ylsulfanyl]ethyl}benzoic acid | Purity: 88.0% | [577] |
| 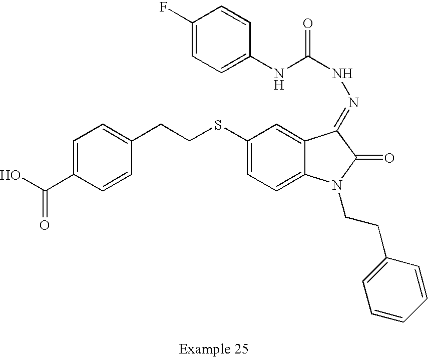 | 4-{2-[3((4-fluoroanilino- carbonyl)hydrazono)-2-oxo-1- phenethyl-2,3-dihydro-1H-indol-5- ylsulfanyl]ethyl}benzoic acid | Purity: 71.0% | [583] +10% syn |
| 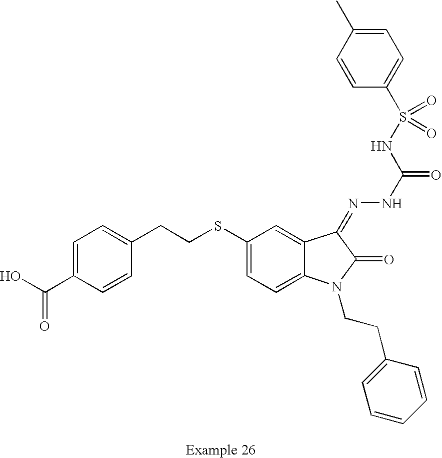 | 4-{2-[3-((4-methylbenzene- sulfonamido)carbonylhydrazono)- 2-oxo-1-phenethyl-2,3-dihydro-1H- indol-5-ylsulfanyl]ethyl}benzoic acid | Purity: 74.5% | [643] +17% ND |
| 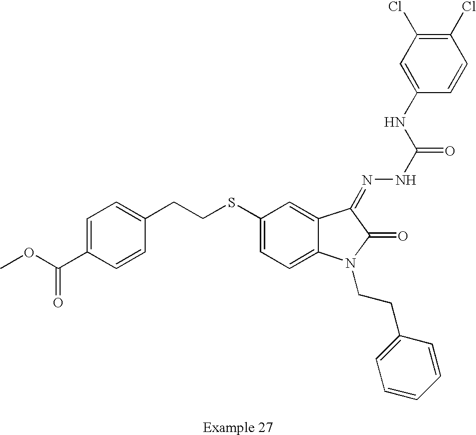 | methyl 4-{2-[3((3,4-dichloro- anilinocarbonyl)hydrazono)-2-oxo- 1-1-phenethyl-2,3-dihydro-1H- indol-5-ylsulfanyl]ethyl}benzoate | Purity: 81.0% | [647] |
| 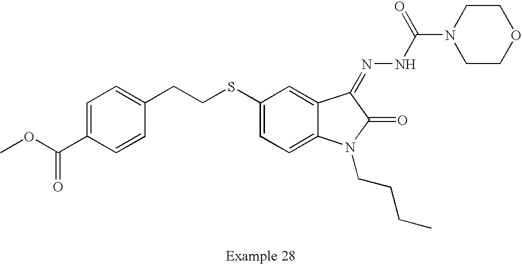 | methyl 4-(2-{1-Butyl-3- [(morpholine-4-carbonyl) hydrazono]-2-oxo-2,3-dihydro-1H- indol-5-ylsulfanyl}ethyl)benzoate | Purity: 89.0% | [525] |
| 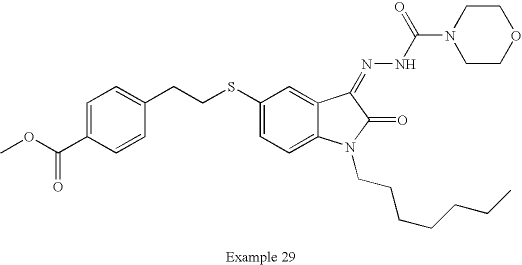 | methyl 4-(2-{1-Heptyl-3- [(morpholine-4-carbonyl) hydrazono]-2-oxo-2,3-dihydro-1H- indol-5-ylsulfanyl}ethyl)benzoate | Purity: 76.0% | [567] +12% ester Et |
| 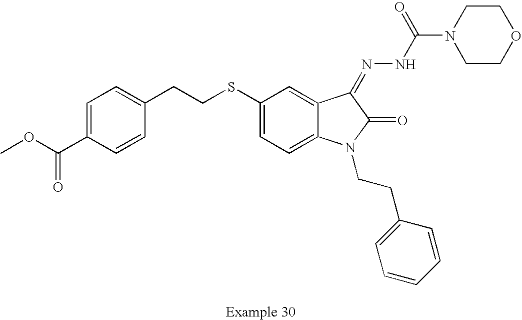 | methyl 4-(2-{3-[(morpholine-4- carbonyl)hydrazono]-2-oxo-1- phenethyl-2,3-dihydro-1H-indol-5- ylsulfanyl}ethyl)benzoate | Purity: 81.0% | [573] |
| 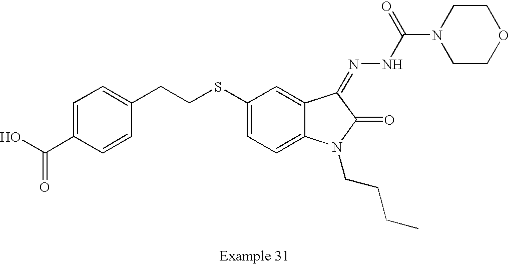 | 4-(2-{1-butyl-3-[(morpholine-4- carbonyl)hydrazono]-2-oxo-2,3- dihydro-1H-indol-5-ylsulfanyl}ethyl)benzoic acid | Purity: 98.0% | [511] |
| 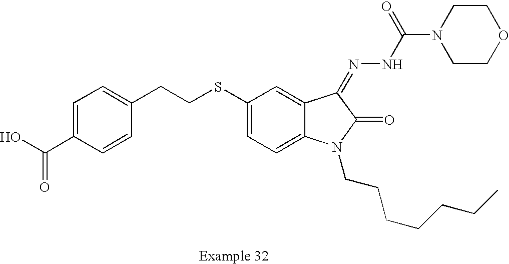 | 4-(2-{1-heptyl-3-[(morpholine-4- carbonyl)hydrazono]-2-oxo-2,3- dihydro-1H-indol-5-ylsulfanyl}ethyl)benzoic acid | Purity: 78.0% | [553] |
| 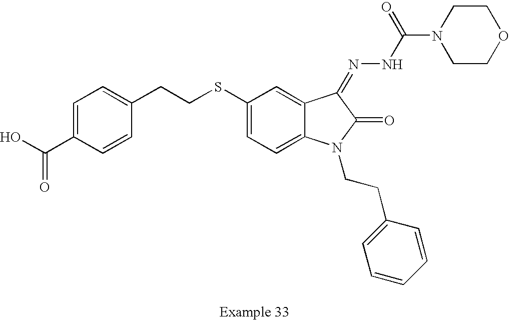 | 4-(2-{3-[(morpholine-4-carbonyl) hydrazono]-2-oxo-1-phenethyl-2,3- dihydro-1H-indol-5-ylsulfanyl}ethyl)benzoic acid | Purity: 89.5% | [559] |
| 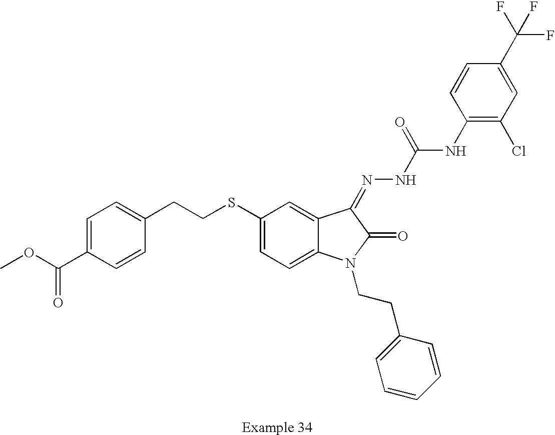 | methyl 4-{2-[3-((2-chloro-4- trifluoromethylanilinocarbonyl) hydrazono)-2-oxo-1-phenethyl-2,3- dihydro-1H-indol-5-ylsulfanyl]ethyl}benzoate | Purity: 75.0% | [681] |
| 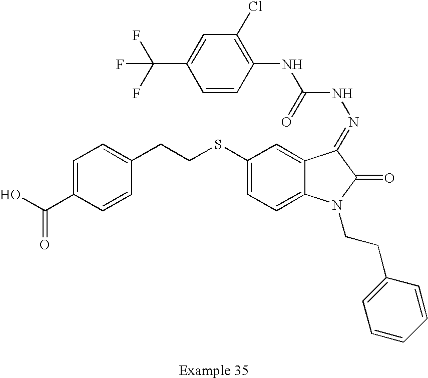 | 4-{2-[3((2-chloro-4-trifluoro- methylanilinocarbonyl)hydrazono)- 2-oxo-1-phenethyl-2,3-dihydro-1H- indol-5-ylsulfanyl]ethyl}benzoic acid | Purity: 84.0% | [667] +10% syn |
| 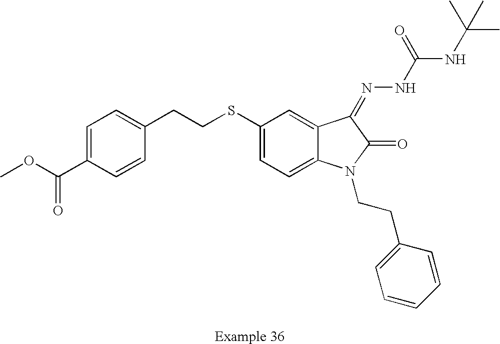 | methyl 4-{2-[3((tert-butylamino) carbonylhydrazono)-2-oxo-1- phenethyl-2,3-dihydro-1H-indol-5- ylsulfanyl]ethyl}benzoate | Purity: 67.0% | [559] |
| 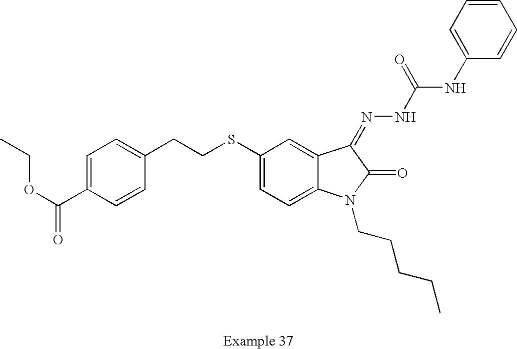 | ethyl 4-{2-[3(((anilinocarbonyl) hydrazono)-2-oxo-1-pentyl-2,3- dihydro-1H-indol-5-ylsulfanyl]ethyl}benzoate | Purity: 99.0% | [558] |
| 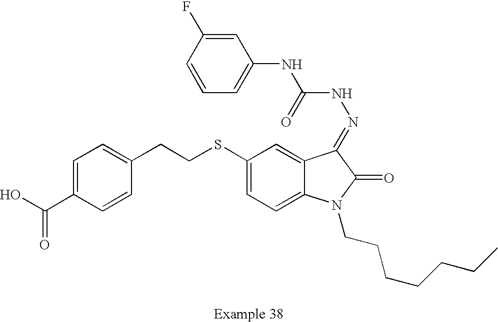 | 4-{2-[3-((3-fluoroanilinocarbonyl) hydrazono)-2-oxo-1-heptyl-2,3- dihydro-1H-indol-5-ylsulfanyl]ethyl}benzoic acid | Purity: 80.0% | |
| 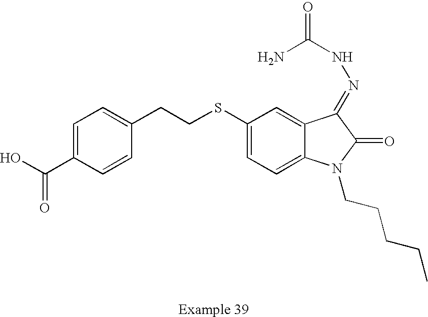 | 4-{2-[3((aminocarbonyl) hydrazono)-2-oxo-1-pentyl-2,3- dihydro-1H-indol-5- ylsulfanyl]ethyl}benzoic acid | 83.00% | 455 +17% [394] |
| 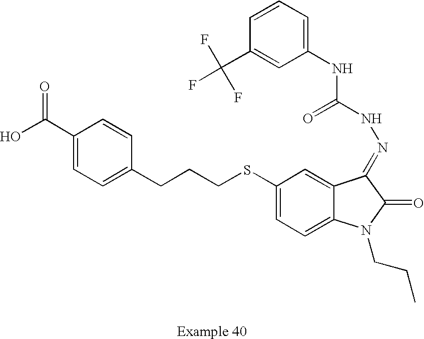 | 4-[3-(3-((3-trifluoromethyl- anilinocarbonyl)hydrazono)-2-oxo- 1-propyl-2,3-dihydro-1H-indol-5- ylsulfanyl)propyl]benzoic acid | 83.00% | 585 |
| 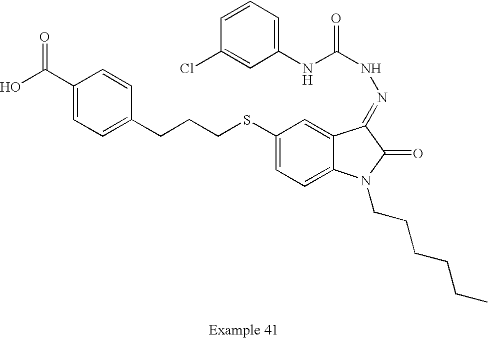 | 4-[3-(3-((3-chloroanilinocarbonyl) hydrazono)-2-oxo-1-hexyl-2,3- dihydro-1H-indol-5- ylsulfanyl)propyl]benzoic acid | 89.00% | 593 |
| 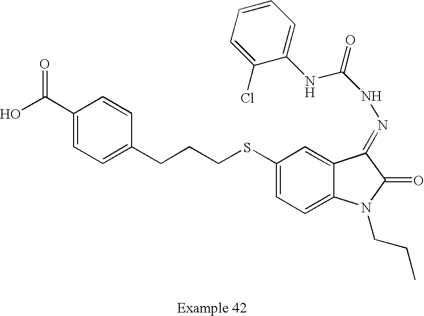 | 4-[3-(3-((2-chloroanilinocarbonyl) hydrazono)-2-oxo-1-propyl-2,3- dihydro-1H-indol-5- ylsulfanyl)propyl]benzoic acid | 86.00% | 551 +14% syn |
| 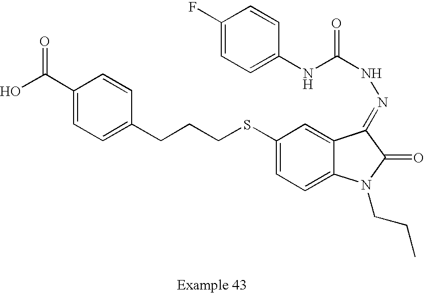 | 4-[3-(3-((4-fluoroanilinocarbonyl) hydrazono)-2-oxo-1-propyl-2,3- dihydro-1H-indol-5- ylsulfanyl)propyl]benzoic acid | 93.00% | 535 |
| 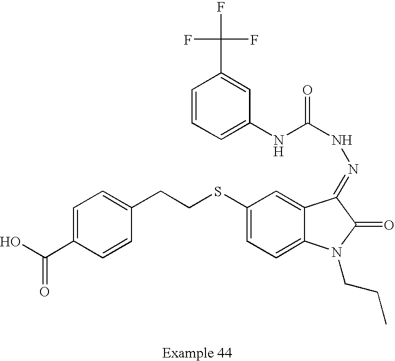 | 4-{2-[3-((3-trifluoromethyl- anilinocarbonyl)hydrazono)-2-oxo- 1-propyl-2,3-dihydro-1H-indol-5- ylsulfanyl]ethyl}benzoic acid | 78.00% | 571 +11% syn |
| 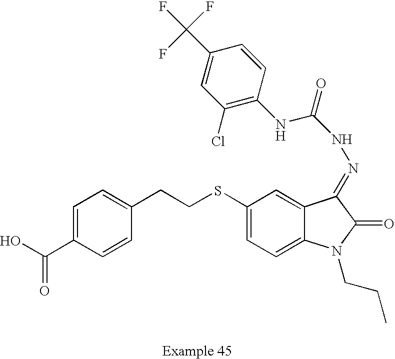 | 4-{2-[3-((2-chloro-3-trifluoro- methylanilinocarbonyl)hydrazono)- 2-oxo-1-propyl-2,3-dihydro-1H- indol-5-ylsulfanyl]ethyl}benzoic acid | 80.00% | 605 +11% syn |
| 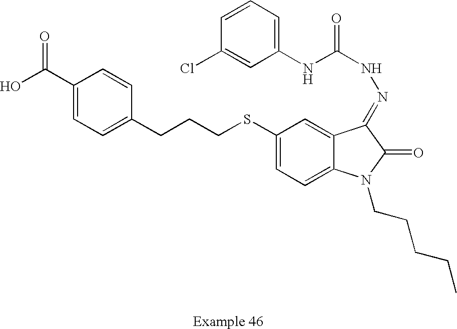 | 4-[3-(3-((3-chloroanilinocarbonyl) hydrazono)-2-oxo- 1-pentyl-2,3-dihydro-1H-indol-5- ylsulfanyl)propyl]benzoic acid | 92.00% | 579 |
| 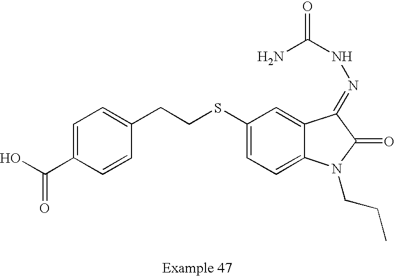 | 4-{2-[3((aminocarbonyl) hydrazono)-2-oxo-1-propyl-2,3- dihydro-1H-indol-5- ylsulfanyl]ethyl}benzoic acid | 94.00% | 426 |
| 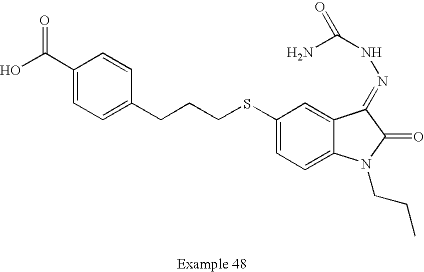 | 4-{3-[3((aminocarbonyl) hydrazono)-2-oxo-1-propyl-2,3- dihydro-1H-indol-5- ylsulfanyl]propyl}benzoic acid | 93.00% | 441 |
| 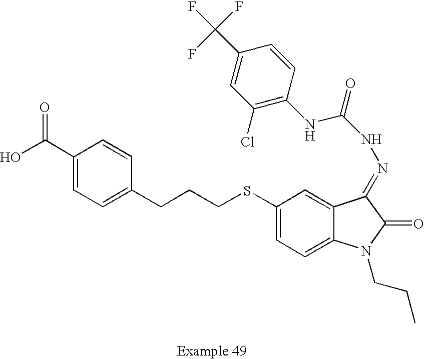 | 4-[3-(3-((2-chloro-4-trifluoro- anilinocarbonyl)hydrazono)-2-oxo- 1-propyl-2,3-dihydro-1H-indol-5- ylsulfanyl)propyl]benzoic acid | 74.00% | 619 |
| 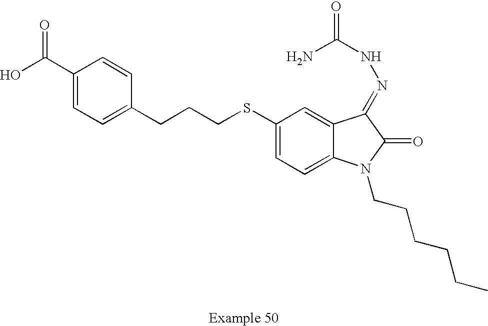 | 4-{3-[3((aminocarbonyl) hydrazono)-2-oxo-1-hexyl-2,3- dihydro-1H-indol-5- ylsulfanyl]propyl}benzoic acid | 83.00% | 483 |
| 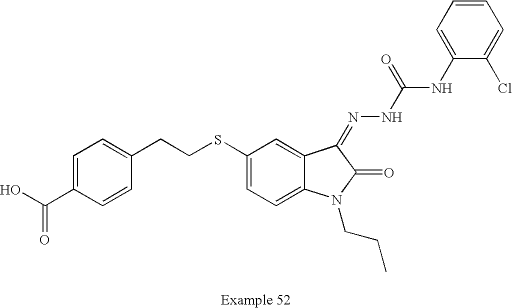 | 4-{2-[3-((2-chloroanilinocarbonyl) hydrazono)-2-oxo-1-propyl-2,3- dihydro-1H-indol-5- ylsulfanyl]ethyl}benzoic acid | 91.00% | 537 |
| 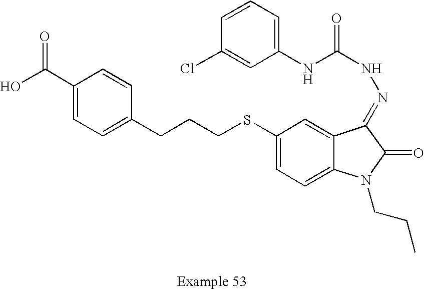 | 4-[3-(3-((3-chloroanilinocarbonyl) hydrazono)-2-oxo-1-propyl-2,3- dihydro-1H-indol-5- ylsulfanyl)propyl]benzoic acid | 84.00% | 551 |
| 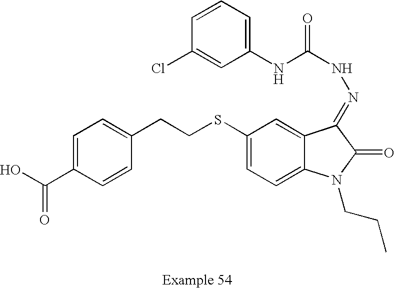 | 4-{2-[3-(3-chlororanilinocarbonyl- hydrazono-2-oxo-1-propyl-2,3- dihydro-1H-indol-5- ylsulfanyl]ethyl}benzoic acid | 93.00% | 537 |
| 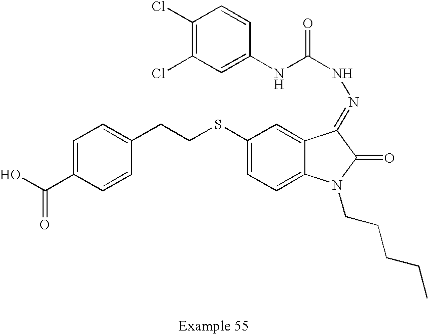 | 4-{2-[3-((3,4-dichloroanilino- carbonyl)hydrazono)-2-oxo-1- pentyl-2,3-dihydro-1H-indol-5- ylsulfanyl]ethyl}benzoic acid | 84.00% | 600 +16% [614] |
| 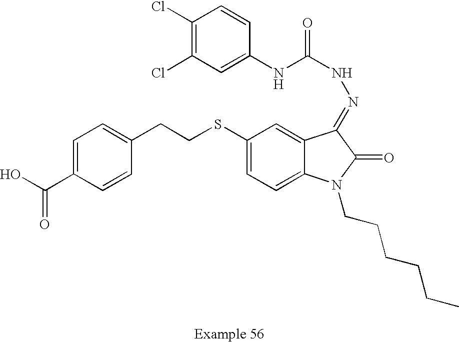 | 4-{2-[3-((3,4-dichloroanilino- carbonyl)hydrazono)-2-oxo-1- hexyl-2,3-dihydro-1H-indol-5- ylsulfanyl]ethyl}benzoic acid | 79.00% | 614 |
| 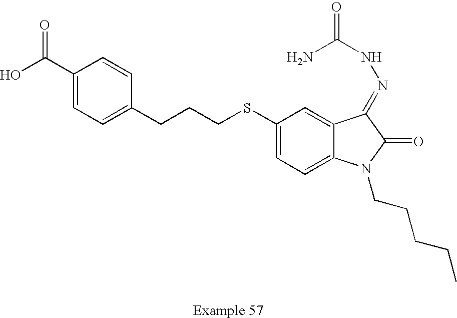 | 4-{3-[3((aminocarbonyl) hydrazono)-2-oxo-1-pentyl-2,3- dihydro-1H-indol-5- ylsulfanyl]propyl}benzoic acid | 82.00% | 469 |
| 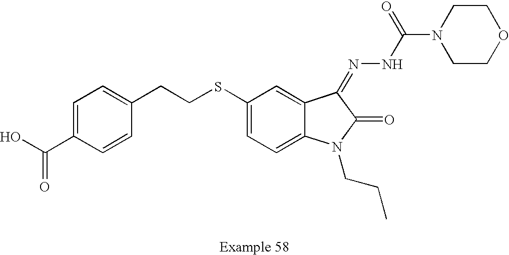 | 4-(2-{3-[(morpholine-4-carbonyl) hydrazono]-2-oxo-1-propyl-2,3- dihydro-1H-indol-5- ylsulfanyl}ethyl)benzoic acid | 83.00% | 497 +12% [571] |
| 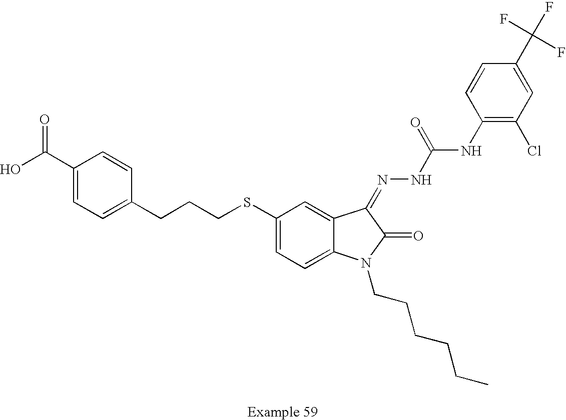 | 4-[3-(3-((2-chloro-4-trifluoro- anilinocarbonyl)hydrazono)-2-oxo- 1-hexyl-2,3-dihydro-1H-indol-5- ylsulfanyl)propyl]benzoic acid | 80.00% | 661 +10% [440] |
| 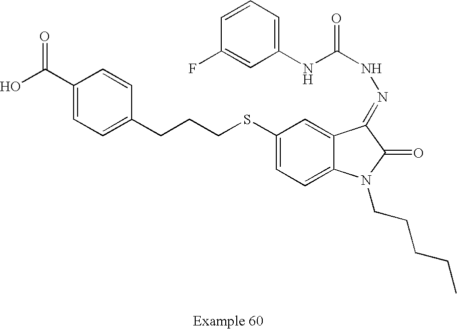 | 4-[3-(3-((3-fluoroanilinocarbonyl) hydrazono)-2-oxo-1-pentyl-2,3- dihydro-1H-indol-5- ylsulfanyl)propyl]benzoic acid | 88.00% | 563 |
| 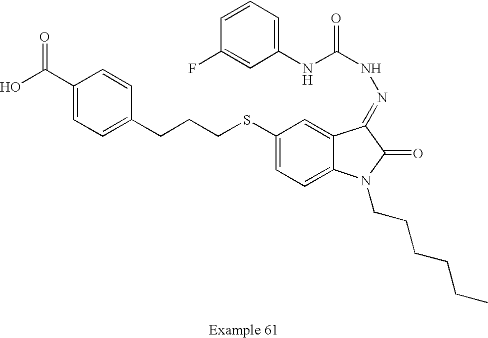 | 4-[3-(3-((3-fluoroanilinocarbonyl) hydrazono)-2-oxo-1-hexyl-2,3- dihydro-1H-indol-5- ylsulfanyl)propyl]benzoic acid | 86.00% | 577 |
| 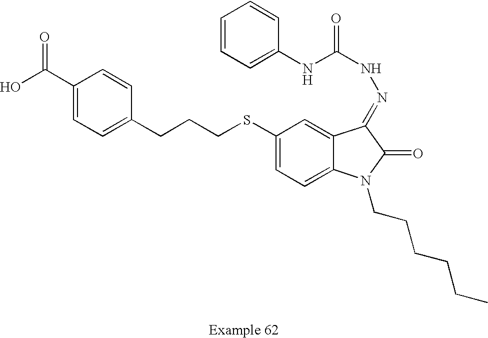 | 4-[3-(3-((anilinocarbonyl) hydrazono)-2-oxo-1- hexyl-2,3-dihydro-1H-indol-5- ylsulfanyl)propyl]benzoic acid | 89.00% | 559 |
| 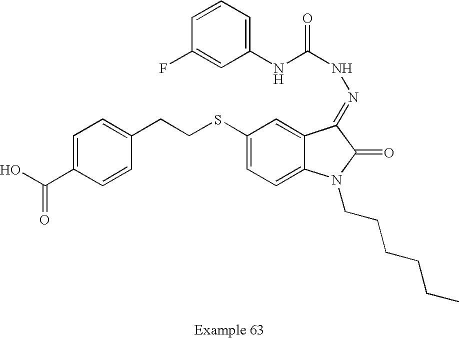 | 4-[3-(3-((3-fluoroanilinocarbonyl) hydrazono)-2-oxo-1-hexyl-2,3- dihydro-1H-indol-5- ylsulfanyl)ethyl]benzoic acid | 74.00% | 563 +10% syn |
| 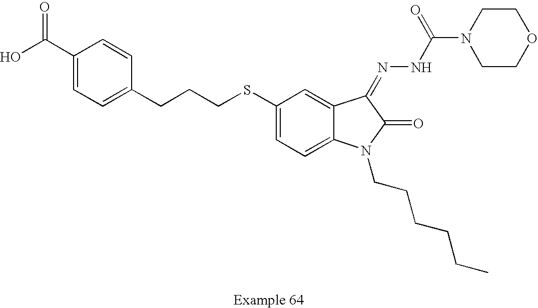 | 4-(3-{1-hexyl-3-[(morpholine-4- carbonyl)hydrazono]-2-oxo-2,3- dihydro-1H-indol-5- ylsulfanyl}propyl)benzoic acid | 81.00% | 553 |
| 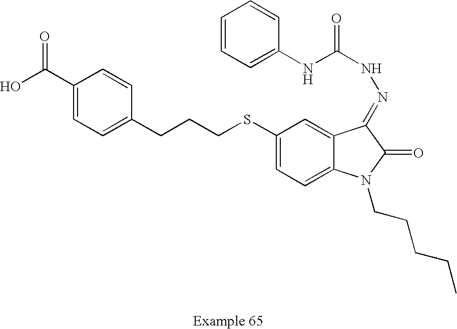 | 4-[3-(3-((anilinocarbonyl) hydrazono)-2-oxo-1- pentyl-2,3-dihydro-1H-indol-5- ylsulfanyl)propyl]benzoic acid | 87.00% | 545 +10% syn |
| 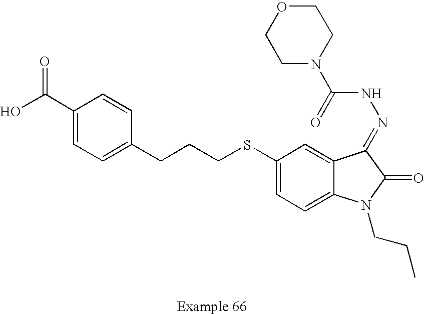 | 4-(3-{3-[(morpholine-4-carbonyl) hydrazono]-2-oxo-1-propyl-2,3- dihydro-1H-indol-5- ylsulfanyl}propyl)benzoic acid | 74.00% | 511 |
| 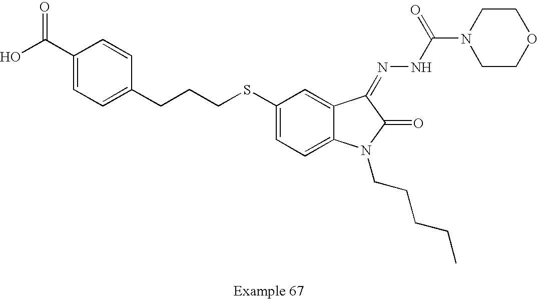 | 4-(3-{3-[(morpholine-4-carbonyl) hydrazono]-2-oxo-1-pentyl-2,3- dihydro-1H-indol-5- ylsulfanyl}propyl)benzoic acid | 83.00% | 539 |
| 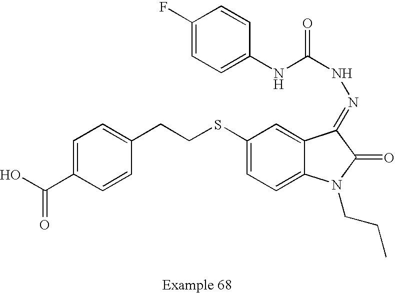 | 4-{2-[3-((4-fluoroanilinocarbonyl) hydrazono)-2-oxo-1-propyl-2,3- dihydro-1H-indol-5- ylsulfanyl]ethyl}benzoic acid | 97.00% | 521 |
| 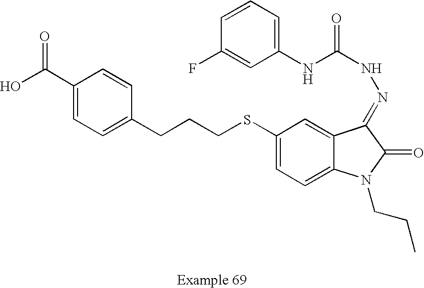 | 4-[3-(3-((3-fluoroanilinocarbonyl) hydrazono)-2-oxo-1-propyl-2,3- dihydro-1H-indol-5- ylsulfanyl)propyl]benzoic acid | 81.00% | 535 |
| 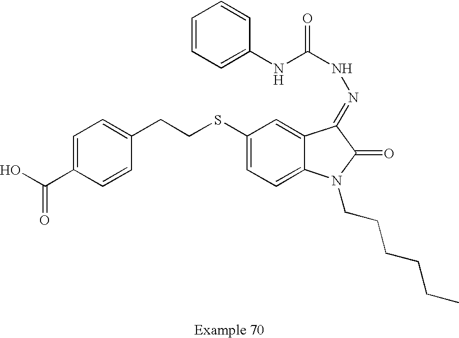 | 4-{2-[3-((anilinocarbonyl) hydrazono)-2-oxo-1-hexyl-2,3- dihydro-1H-indol-5- ylsulfanyl]ethyl}benzoic acid | 81.00% | 545 +16% syn |
| 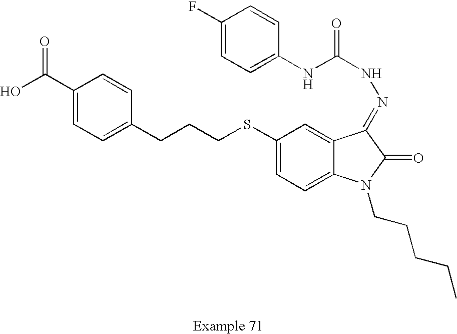 | 4-[3-(3-((4-fluoroanilinocarbonyl) hydrazono)-2-oxo-1-pentyl-2,3- dihydro-1H-indol-5- ylsulfanyl)propyl]benzoic acid | 83.00% | 563 +12% syn |
| 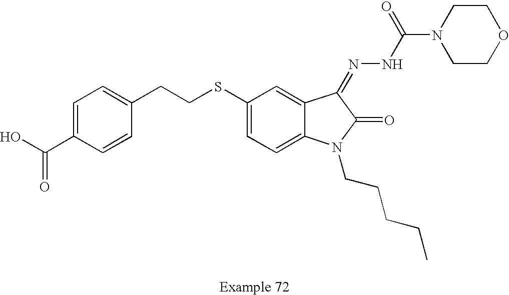 | 4-(2-{3-[(morpholine-4-carbonyl) hydrazono]-2-oxo-1-pentyl-2,3- dihydro-1H-indol-5- ylsulfanyl}ethyl)benzoic acid | 90.00% | 525 |
| 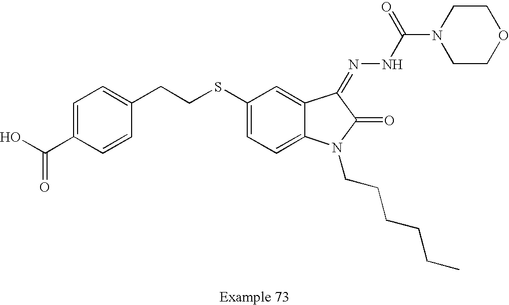 | 4-(2-{1-hexyl-3-[(morpholine-4- carbonyl)hydrazono]-2-oxo-2,3- dihydro-1H-indol-5- ylsulfanyl}ethyl)benzoic acid | 87.00% | 539 |
| 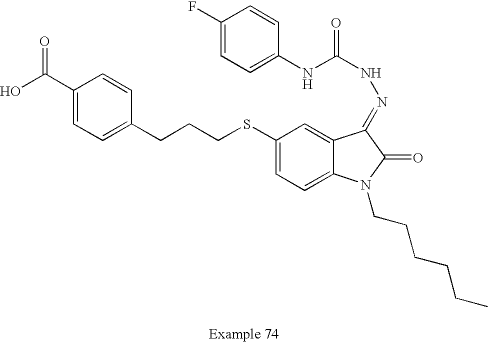 | 4-[3-(3-((4-fluoroanilinocarbonyl) hydrazono)-2-oxo-1-hexyl-2,3- dihydro-1H-indol-5- ylsulfanyl)propyl]benzoic acid | 84.00% | 577 +12% syn |
| 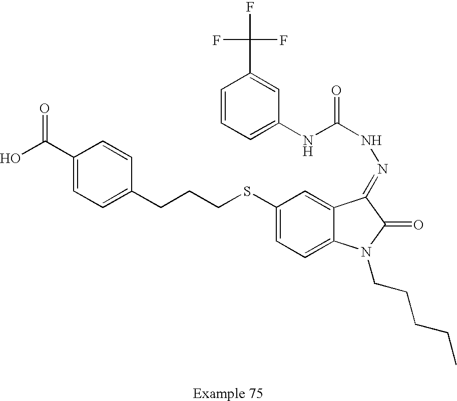 | 4-[3-(3-((3-trifluoromethylanilino- carbonyl)hydrazono)-2-oxo-1- pentyl-2,3-dihydro-1H-indol-5- ylsulfanyl)propyl]benzoic acid | 93.00% | 613 |
| 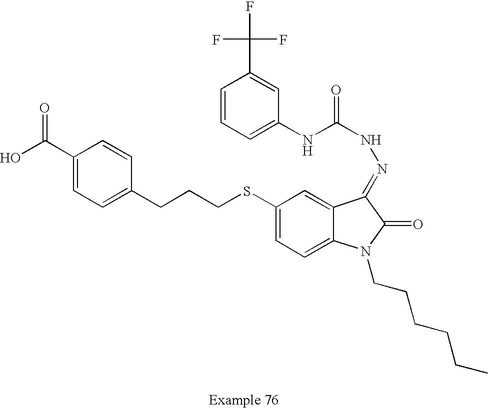 | 4-[3-(3-((3-trifluoromethylanilino- carbonyl)hydrazono)-2-oxo-1- hexyl-2,3-dihydro-1H-indol-5- ylsulfanyl)propyl]benzoic acid | 95.00% | 627 |
| 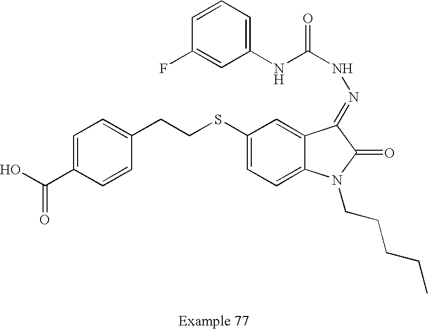 | 4-{2-[3-((3-fluoroanilinocarbonyl) hydrazono)-2-oxo-1-pentyl-2,3- dihydro-1H-indol-5- ylsulfanyl]ethyl}benzoic acid | 87.00% | 549 |
| 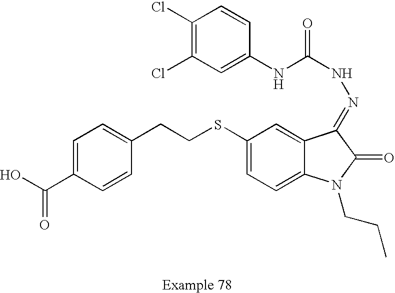 | 4-{2-[-((3,4-dichloroanilino- carbonyl)hydrazono)-2-oxo-1- propyl-2,3-dihydro-1H-indol-5- ylsulfanyl]ethyl}benzoic acid | 82.00% | 571 |
| 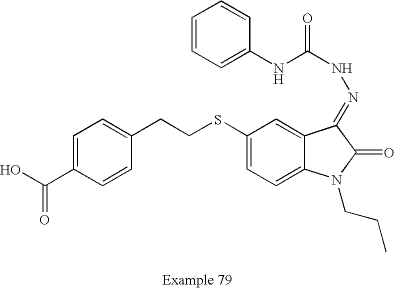 | 4-{2-[3-((anilinocarbonyl) hydrazono)-2-oxo-1-propyl-2,3- dihydro-1H-indol-5- ylsulfanyl]ethyl}benzoic acid | 95.00% | 503 |
| 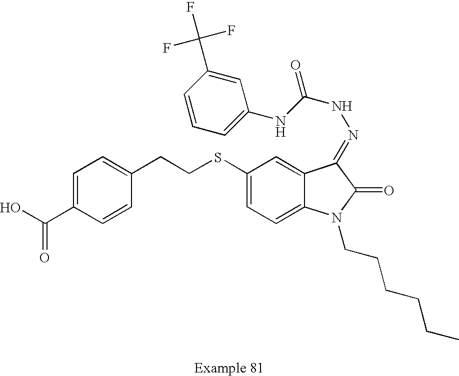 | 4-{2-[3-((3-trifluoromethylanilino) carbonylhydrazono)-2-oxo-1- hexyl-2,3-dihydro-1H-indol-5- ylsulfanyl]ethyl}benzoic acid | 76.00% | 613 +17% syn |
| 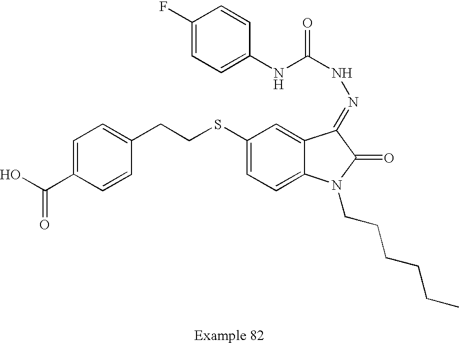 | 4-{2-[3-((4-fluoroanilino)carbonyl- hydrazono)-2-oxo-1-hexyl-2,3- dihydro-1H-indol-5- ylsulfanyl]ethyl}benzoic acid | 94.00% | 563 |
| 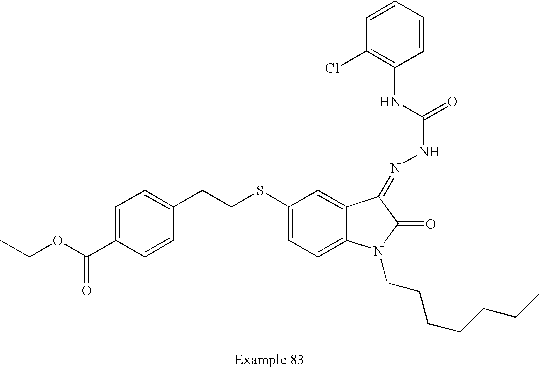 | ethyl 4-{2-[3-((2-chloroanilino- carbonyl)hydrazono)-2-oxo-1- heptyl-2,3-dihydro-1H-indol-5- ylsulfanyl]ethyl}benzoate | 74.00% | 621 |
| 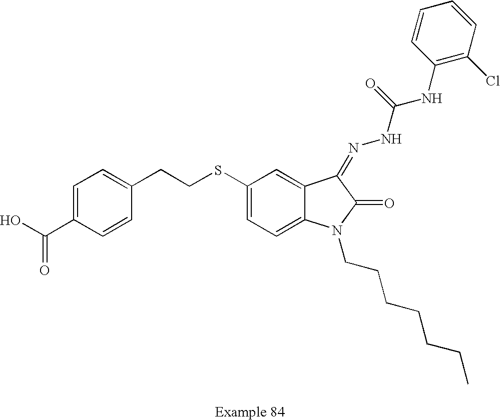 | 4-{2-[3-((2-chloroanilino) carbonylhydrazono)-2-oxo-1- heptyl-2,3-dihydro-1H-indol-5- ylsulfanyl]ethyl}benzoic acid | 96.00% | 593 |

### EXAMPLE 86

### Cross-Curve PPAR Transactivation Tests

The activation of the receptors with an agonist (activator) in HeLN cells leads to the expression of a reporter gene, luciferase, which, in the presence of a substrate, generates light. The modulation of the receptors is measured by quantifying the luminescence produced after incubating the cells in the presence of a reference agonist. The ligands displace the agonist from its site. The measurement of the activity is performed by quantifying the light produced. This measurement makes it possible to determine the modulatory activity of the compounds according to the invention by determining the constant that is the affinity of the molecule for the receptor. Since this value can fluctuate depending on the basal activity and the expression of the receptor, it is referred to as Kd apparent (KdApp in nM).

To determine this constant, “cross curves” of the test product against a reference agonist are produced in a 96-well plate: 10 concentrations of the test product plus a concentration 0 are arranged in a line, and 7 concentrations of the agonist plus a concentration 0 are arranged in a column. This is 88 measurement points for 1 product and 1 receptor. The remaining 8 wells are used for repeatability controls.

In each well, the cells are in contact with a concentration of the test product and a concentration of the reference agonist, 2-(4-{2-[3-(2,4-difluorophenyl)-1-heptylureido]ethyl}phenylsulfanyl)-2-methylpropionic acid for PPARα, {2-methyl-4-[4-methyl-2-(4-trifluoromethylphenyl)thiazol-5-ylmethylsulfanyl]phenoxy}acetic acid for PPARδ and 5-{4-[2-(methylpyrid-2-ylamino)ethoxy]benzyl}thiazolidine-2,4-dione for PPARγ. Measurements are also taken for total agonist controls with the same products.

The HeLN cell lines used are stable transfectants containing the plasmids ERE-βGlob-Luc-SV-Neo (reporter gene) and PPAR (α, δ, γ) Gal-hPPAR. These cells are inoculated into 96-well plates at a rate of 10 000 cells per well in 100 μl of DMEM medium without phenol red and supplemented with 10% of defatted calf serum. The plates are then incubated at 37° C. and 7% CO₂ for 16 hours.

The various dilutions of the test products and of the reference ligand are added at a rate of 5 μl per well. The plates are then incubated for 18 hours at 37° C. and 7% CO₂. The culture medium is removed by turning over and 100 μl of a 1:1 PBS/luciferin mixture are added to each well. After 5 minutes, the plates are read by the luminescence detector.

These cross curves make it possible to determine the AC50 values (concentration at which 50% activation is observed) of the reference ligand at various concentrations of test product. These AC50 values are used to calculate the Schild regression by plotting a straight line corresponding to the Schild equation (“quantitation in receptor pharmacology” Terry P. Kenakin, Receptors and Channels, 2001, 7, 371-385), which allows the Kd app values (in nM) to be obtained.

Transactivation Results:

| | PPAR | PPARs | PPAR |
|---|---|---|---|
| | alpha | delta | gamma |
| | Kd app | Kd app | Kd app |
| Compounds | (nM) | (in nM) | (in nM) |
| Reference 1: 2-(4-{2-[3-(2,4- | 200 | n.a. | n.a. |
| difluorophenyl)-1- | | | |
| heptylureido]ethyl}phenylsul- | | | |
| fanyl)2-methylpropionic acid | | | |
| Reference 2: {2-methyl-4-[4- | n.a. | 10 | n.a. |
| methyl-2-(4-trifluoromethyl- | | | |
| phenyl)thiazol-5- | | | |
| ylmethylsulfanyl]phenoxy}acetic | | | |
| acid | | | |
| Reference 3: 5-{4-[2-(methyl- | n.a | n.a. | 30 |
| pyrid-2- | | | |
| ylamino)ethoxy]benzyl}- | | | |
| thiazolidine-2,4-dione | | | |
| Example 14 | 2000 | 120 | 1000 |
| Example 16 | 2000 | 4000 | 500 |
| Example 17 | 250 | n.a. | 1000 |
| Example 18 | 2000 | 250 | 500 |
| Example 20 | 2000 | 500 | 1000 |
| Example 22 | 2000 | 60 | 1000 |
| Example 35 | 8000 | 500 | 250 |
| Example 62 | n.a. | 500 | 4000 |
| Example 74 | 4000 | 250 | 4000 |
| Example 84 | 500 | 30 | 1000 |

n.a. means not active

### EXAMPLE 87

### Compositions

Various specific formulations based on the compounds according to the invention are illustrated in this example.

A—Oral Route:

(a) 0.2 g Tablet:

| | |
|---|---|
| Compound of Example 1 | 0.001 g |
| Starch | 0.114 g |
| Dicalcium phosphate | 0.020 g |
| Silica | 0.020 g |
| Lactose | 0.030 g |
| Talc | 0.010 g |
| Magnesium stearate | 0.005 g |

(b) Drinkable Suspension in 5 ml Ampules:

| | |
|---|---|
| Compound of Example 5 | 0.001 g |
| Glycerol | 0.500 g |
| 70% sorbitol | 0.500 g |
| Sodium saccharinate | 0.010 g |
| Methyl parahydroxybenzoate | 0.040 g |
| Flavoring | qs |
| Purified water | qs 5 ml |

(c) 0.8 g Tablet:

| | |
|---|---|
| Compound of Example 20 | 0.500 g |
| Pregelatinized starch | 0.100 g |
| Micro-crystalline cellulose | 0.115 g |
| Lactose | 0.075 g |
| Magnesium stearate | 0.010 g |

(d) Drinkable Suspension in 10 ml Ampules:

| | |
|---|---|
| Compound of Example 45 | 0.200 g |
| Glycerol | 1.000 g |
| 70% sorbitol | 1.000 g |
| Sodium saccharinate | 0.010 g |
| Methyl parahydroxybenzoate | 0.080 g |
| Flavoring | qs |
| Purified water | qs 10 ml |

B—Topical Route:

(a) Ointment:

| | |
|---|---|
| Compound of Example 62 | 0.020 g |
| Isopropyl myristate | 81.700 g |
| Fluid petroleum jelly oil | 9.100 g |
| Silica (“Aerosil 200” marketed by Degussa) | 9.180 g |

(b) Ointment:

| | | |
|---|---|---|
| Compound of Example 72 | 0.300 | g |
| White petroleum jelly codex | qs 100 | g |

(c) Nonionic Water-in-Oil Cream:

| | | |
|---|---|---|
| Compound of Example 9 | 0.100 | g |
| Mixture of emulsifying lanolin alcohols, waxes | 39.900 | g |
| and oils (“Anhydrous Eucerin” marketed by BDF) | | |
| Methyl parahydroxybenzoate | 0.075 | g |
| Propyl parahydroxybenzoate | 0.075 | g |
| Sterile demineralized water | qs 100 | g |

(d) Lotion:

| | | |
|---|---|---|
| Compound of Example 37 | 0.100 | g |
| Polyethylene glycol (PEG 400) | 69.900 | g |
| 95% ethanol | 30.000 | g |

(e) Hydrophobic Ointment:

| | | |
|---|---|---|
| Compound of Example 54 | 0.300 | g |
| Isopropyl myristate | 36.400 | g |
| Silicone oil (“Rhodorsil 47 V 300” marketed | 36.400 | g |
| by Rhone-Poulenc) | | |
| Beeswax | 13.600 | g |
| Silicone oil (“Abil 300.000 cSt” marketed | qs 100 | g |
| by Goldschmidt) | | |

(f) Nonionic Oil-in-Water Cream:

| | | |
|---|---|---|
| Compound of Example 84 | 1.000 | g |
| Cetyl alcohol | 4.000 | g |
| Glyceryl monostearate | 2.500 | g |
| PEG 50 stearate | 2.500 | g |
| Shea butter | 9.200 | g |
| Propylene glycol | 2.000 | g |
| Methyl parahydroxybenzoate | 0.075 | g |
| Propyl parahydroxybenzoate | 0.075 | g |
| Sterile demineralized water | qs 100 | g |

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A biaromatic compound having the following general formula (I): 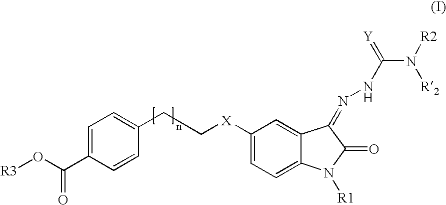
in which:
R₁ is an alkyl radical having from 1 to 10 carbon atoms, an aralkyl radical or an aryl radical;
R2 is a hydrogen atom, a linear or branched alkyl radical having from 1 to 7 carbon atoms, a substituted or unsubstituted aryl radical, a substituted or unsubstituted phenylsulfonyl radical, a substituted or unsubstituted heteroaryl radical, an aralkyl radical or a heterocyclic radical;
R′2 is a hydrogen atom;
with the proviso that R2 and R′2 can together form a heterocycle,
R3 is a hydrogen atom, an alkyl radical having from 1 to 3 carbon atoms, a polyether radical, an aryl radical, an aralkyl radical, a heteroaryl radical, a monohydroxyalkyl radical or a polyhydroxyalkyl radical;
X is S, CH₂, N or O;
Y is an oxygen or sulfur atom;
n is 1 or 2;
and the geometrical isomers and also the salts thereof.

2. The biaromatic compound as defined by claim 1, wherein the double bond bonding the nitrogen to the ring present in the compounds of general formula (I) is in the syn or anti configuration.

3. The biaromatic compound as defined by claim 1, being a salt of an alkali metal or alkaline-earth metal, a zinc salt or a salt of an organic amine.

4. The biaromatic compound as defined by claim 1, having at least one methyl, ethyl, n-propyl, n-butyl, tert-butyl, n-pentyl, n-hexyl or n-heptyl radical substituent.

5. The biaromatic compound as defined by claim 1, having at least one phenyl radical substituent, which may be mono- or disubstituted with one or more atoms or radicals selected from the group consisting of a halogen atom, a CF₃ radical and a methyl radical.

6. The biaromatic compound as defined by claim 1, wherein formula (I), R2 is a phenylsulfonyl radical substituted with a methyl group.

7. The biaromatic compound as defined by claim 1, having at least one benzyl radical and/or phenethyl radical substituent.

8. The biaromatic compound as defined by claim 1, having at least one heteroaryl radical substituent selected from the group consisting of an aryl radical interrupted with one or more hetero atoms, optionally substituted with at least one halogen atom, an alkyl radical having from 1 to 12 carbon atoms, an alkoxy radical having from 1 to 7 carbon atoms, an aryl radical, a nitro function, a polyether radical, a heteroaryl radical, a benzoyl radical, an alkyl ester group, a carboxylic acid, a hydroxyl group optionally protected with an acetyl or benzoyl group or an amino function optionally protected with an acetyl or benzoyl group or optionally substituted with at least one alkyl radical having from 1 to 12 carbon atoms.

9. The biaromatic compound as defined by claim 1, having at least one morpholino radical substituent.

10. The biaromatic compound as defined by claim 1, wherein formula (I), R3 is a methoxymethoxy, ethoxymethoxy or methoxyethoxymethoxy radical.

11. The biaromatic compound as defined by claim 1, wherein foruma (I), R3 is a 2-hydroxyethyl, 2-hydroxypropyl or 3-hydroxypropyl radical.

12. The biaromatic compound as defined by claim 1, wherein formula (I), R3 is a 2,3-dihydroxypropyl, 2,3,4-trihydroxybutyl and 2,3,4,5-tetrahydroxypentyl radical.

13. The biaromatic compound as defined by claim 1, having at least one halogen atom substituent.

14. The biaromatic compound as defined by claim 1, selected form the group consisting of:
1. 4-{2-[3-((anilinocarbonyl)hydrazono)-2-oxo-1-pentyl-2,3-dihydro-1H-indol-5-ylsulfanyl]ethyl}benzoic acid;
2. 4-{2-[3-((anilinocarbonyl)hydrazono)-2-oxo-1-pentyl-2,3-dihydro-1H-indol-5-ylsulfanyl]ethyl}benzoic acid;
3. 4-{2-[3-((anilinocarbonyl)hydrazono)-2-oxo-1-methyl-2,3-dihydro-1H-indol-5-ylsulfanyl]ethyl}benzoic acid;
4. 4-{2-[3-((anilinocarbonyl)hydrazono)-2-oxo-1-pentyl-2,3-dihydro-1H-indol-5-ylsulfanyl]ethyl}benzoic acid;
5. 4-[((3Z)-3-{[(benzylamino)carbonothioyl]hydrazono}-2-oxo-1-methyl-2,3-dihydro-1H-indol-5-ylsulfanyl]ethyl}benzoic acid;
6. 4-{2-[3-(benzylaminocarbonothioylhydrazono)-2-oxo-1-pentyl-2,3-dihydro-1H-indol-5-ylsulfanyl]ethyl}benzoic acid;
7. 4-{2-[3-(benzylaminocarbonothioylhydrazono)-2-oxo-1-benzyl-2,3-dihydro-1H-indol-5-ylsulfanyl]ethyl}benzoic acid;
8. 4-{2-[3-((anilinocarbonyl)hydrazono)-2-oxo-1-benzyl-2,3-dihydro-1H-indol-5-ylsulfanyl]ethyl}benzoic acid;
9. 4-{2-[3-(anilinocarbonothioylhydrazono)-2-oxo-1-pentyl-2,3-dihydro-1H-indol-5-ylsulfanyl]ethyl}benzoic acid;
10. 4-{2-[3-(ethylaminocarbonothioylhydrazono)-2-oxo-1-pentyl-2,3-dihydro-1H-indol-5-ylsulfanyl]ethyl}benzoic acid;
11. methyl 4-{2-[3-((4-fluoroanilino)carbonylhydrazono)-2-oxo-1-phenethyl-2,3-dihydro-1H-indol-5-ylsulfanyl]ethyl}benzoate;
12. methyl 4-{2-[3-((anilinocarbonyl)hydrazono)-2-oxo-1-butyl-2,3-dihydro-1H-indol-5-ylsulfanyl]ethyl}benzoate;
13. methyl 4-{2-[3-((anilinocarbonyl)hydrazono)-2-oxo-1-heptyl-2,3-dihydro-1H-indol-5-ylsulfanyl]ethyl}benzoate;
14. 4-{2-[3-((anilinocarbonyl)hydrazono)-2-oxo-1-butyl-2,3-dihydro-1H-indol-5-ylsulfanyl]ethyl}benzoic acid;
15. 4-{2-[3-((anilinocarbonyl)hydrazono)-2-oxo-1-heptyl-2,3-dihydro-1H-indol-5-ylsulfanyl]ethyl}benzoic acid;
16. 4-{2-[3-((anilinocarbonyl)hydrazono)-2-oxo-1-phenethyl-2,3-dihydro-1H-indol-5-ylsulfanyl]ethyl}benzoic acid;
17. methyl 4-{2-[3-((3-fluoroanilinocarbonyl)hydrazono)-2-oxo-1-phenethyl-2,3-dihydro-1H-indol-5-ylsulfanyl]ethyl}benzoate;
18. 4-{2-[3-((3-fluoroanilinocarbonyl)hydrazono)-2-oxo-1-butyl-2,3-dihydro-1H-indol-5-yl-sulfanyl]ethyl}benzoic acid;
19. methyl 4-{2-[3-((3-trifluoromethylanilinocarbonyl)hydrazono)-2-oxo-1-phenethyl-2,3-dihydro-1H-indol-5-ylsulfanyl]ethyl}benzoate;
20. 4-{2-[3-((3-trifluoromethylanilinocarbonyl)hydrazono)-2-oxo-1-heptyl-2,3-dihydro-1H-indol-5-ylsulfanyl]ethyl}benzoic acid;
21. methyl 4-{2-[3((2-chloroanilinocarbonyl)hydrazono)-2-oxo-1-phenethyl-2,3-dihydro-1H-indol-5-ylsulfanyl]ethyl}benzoate.
22. 4-{2-[3((2-chloroanilinocarbonyl)hydrazono)-2-oxo-1-heptyl-2,3-dihydro-1H-indol-5-yl-sulfanyl]ethyl}benzoic acid;
23. 4-{2-[3((2-chloroanilinocarbonyl)hydrazono)-2-oxo-1-phenethyl-2,3-dihydro-1H-indol-5-ylsulfanyl]ethyl}benzoic acid;
24. 4-{2-[3((4-fluoroanilinocarbonyl)hydrazono)-2-oxo-1-heptyl-2,3-dihydro-1H-indol-5-yl-sulfanyl]ethyl}benzoic acid;
25. 4-{2-[3((4-fluoroanilinocarbonyl)hydrazono)-2-oxo-1-phenethyl-2,3-dihydro-1H-indol-5-ylsulfanyl]ethyl}benzoic acid;
26. 4-{2-[3((4-methylbenzenesulfonamido)carbonylhydrazono)-2-oxo-1-phenethyl-2,3-dihydro-1H-indol-5-ylsulfanyl]ethyl}benzoic acid;
27. methyl 4-{2-[3((3,4-dichloroanilinocarbonyl)hydrazono)-2-oxo-1-1-phenethyl-2,3-dihydro-1H-indol-5-ylsulfanyl]ethyl}benzoate;
28. methyl 4-(2-{1-butyl-3-[(morpholine-4-carbonyl)hydrazono]-2-oxo-2,3-dihydro-1H-indol-5-ylsulfanyl}ethyl)benzoate;
29. methyl 4-(2-{1-heptyl-3-[(morpholine4-carbonyl)hydrazono]-2-oxo-2,3-dihydro-1H-indol-5-ylsulfanyl}ethyl)benzoate;
30. methyl 4-(2-{3-[(morpholine4-carbonyl)hydrazono]-2-oxo-1-phenethyl-2,3-dihydro-1H-indol-5-ylsulfanyl}ethyl)benzoate;
31. 4-(2-{1-butyl-3-[(morpholine4-carbonyl)hydrazono]-2-oxo-2,3-dihydro-1H-indol-5-yl-sulfanyl}ethyl)benzoic acid;
32. 4-(2-{1-heptyl-3-[(morpholine-4-carbonyl)hydrazono]-2-oxo-2,3-dihydro-1H-indol-5-ylsulfanyl}ethyl)benzoic acid;
33. 4-(2-{3-[(morpholine4-carbonyl)hydrazono]-2-oxo-1-phenethyl-2,3-dihydro-1H-indol-5-ylsulfanyl}ethyl)benzoic acid;
34. methyl 4-{2-[3-((2-chloro-4-trifluoromethylanilinocarbonyl)hydrazono)-2-oxo-1-phen-ethyl-2,3-dihydro-1H-indol-5-ylsulfanyl]ethyl}benzoate;
35. 4-{2-[3((2-chloro-4-trifluoromethylanilinocarbonyl)hydrazono)-2-oxo-1-phenethyl-2,3-dihydro-1H-indol-5-ylsulfanyl]ethyl}benzoic acid;
36. methyl 4-{2-[3((tert-butylamino)carbonylhydrazono)-2-oxo-1-phenethyl-2,3-dihydro-1H-indol-5-ylsulfanyl]ethyl}benzoate;
37. ethyl 4-{2-[3(((anilinocarbonyl)hydrazono)-2-oxo-1-pentyl-2,3-dihydro-1H-indol-5-ylsulfanyl]ethyl}benzoate;
38. 4-{2-[3-((3-fluoroanilinocarbonyl)hydrazono)-2-oxo-1-heptyl-2,3-dihydro-1H-indol-5-ylsulfanyl]ethyl}benzoic acid;
39. 4-{2-[3((aminocarbonyl)hydrazono)-2-oxo-1-pentyl-2,3-dihydro-1H-indol-5-ylsulfanyl]ethyl}benzoic acid;
40. 4-[3-(3-((3-trifluoromethylanilinocarbonyl)hydrazono)-2-oxo-1-propyl-2,3-dihydro-1H-indol-5-ylsulfanyl)propyl]benzoic acid;
41. 4-[3-(3-((3-chloroanilinocarbonyl)hydrazono)-2-oxo-1-hexyl-2,3-dihydro-1H-indol-5-ylsulfanyl)propyl]benzoic acid;
42. 4-[3-(3-((2-chloroanilinocarbonyl)hydrazono)-2-oxo-1-propyl-2,3-dihydro-1H-indol-5-ylsulfanyl)propyl]benzoic acid;
43. 4-[3-(3-((4-fluoroanilinocarbonyl)hydrazono)-2-oxo-1-propyl-2,3-dihydro-1H-indol-5-ylsulfanyl)propyl]benzoic acid;
44. 4-{2-[3-((3-trifluoromethylanilinocarbonyl)hydrazono)-2-oxo-1-propyl-2,3-dihydro-1H-indol-5-ylsulfanyl]ethyl}benzoic acid;
45. 4-{2-[3-((2-chloro-3-trifluoromethylanilinocarbonyl)hydrazono)-2-oxo-1-propyl-2,3-dihydro-1H-indol-5-ylsulfanyl]ethyl}benzoic acid;
46. 4-[3-(3-((3-chloroanilinocarbonyl)hydrazono)-2-oxo-1-pentyl-2,3-dihydro-1H-indol-5-ylsulfanyl)propyl]benzoic acid;
47. 4-{2-[3((aminocarbonyl)hydrazono)-2-oxo-1-propyl-2,3-dihydro-1H-indol-5-ylsulfanyl]ethyl}benzoic acid;
48. 4-{3-[3((aminocarbonyl)hydrazono)-2-oxo-1-propyl-2,3-dihydro-1H-indol-5-ylsulfanyl]propyl}benzoic acid;
49. 4-[3-(3-((2-chloro-4-trifluoroanilinocarbonyl)hydrazono)-2-oxo-1-propyl-2,3-dihydro-1H-indol-5-ylsulfanyl)propyl]benzoic acid;
50. 4-{3-[3((aminocarbonyl)hydrazono)-2-oxo-1-hexyl-2,3-dihydro-1H-indol-5-ylsulfanyl]propyl}benzoic acid;
51. 4-{2-[3((thiophen-2-ylamine)carbonylhydrazono)-2-oxo-1-pentyl-2,3-dihydro-1H-indol-5-ylsulfanyl]ethyl}benzoic acid;
52. 4-{2-[3-((2-chloroanilinocarbonyl)hydrazono)-2-oxo-1-propyl-2,3-dihydro-1H-indol-5-ylsulfanyl]ethyl}benzoic acid;
53. 4-[3-(3-((3-chloroanilinocarbonyl)hydrazono)-2-oxo-1-propyl-2,3-dihydro-1H-indol-5-ylsulfanyl)propyl]benzoic acid;
54. 4-{2-[3-((3-chlororanilinocarbonyl)hydrazono)-2-oxo-1-propyl-2,3-dihydro-1H-indol-5-ylsulfanyl]ethyl}benzoic acid;
55. 4-{2-[3-((3,4-dichloroanilinocarbonyl)hydrazono)-2-oxo-1-pentyl-2,3-dihydro-1H-indol-5-ylsulfanyl]ethyl}benzoic acid;
56. 4-{2-[3-((3,4-dichloroanilinocarbonyl)hydrazono)-2-oxo-1-hexyl-2,3-dihydro-1H-indol-5-ylsulfanyl]ethyl}benzoic acid;
57. 4-{3-[3((aminocarbonyl)hydrazono)-2-oxo-1-pentyl-2,3-dihydro-1H-indol-5-ylsulfanyl]propyl}benzoic acid;
58. 4-(2-{3-[(morpholine-4-carbonyl)hydrazono]-2-oxo-1-propyl-2,3-dihydro-1H-indol-5-ylsulfanyl}ethyl)benzoic acid;
59. 4-[3-(3-((2-chloro-4-trifluoroanilinocarbonyl)hydrazono)-2-oxo-1-hexyl-2,3-dihydro-1H-indol-5-ylsulfanyl)propyl]benzoic acid;
60. 4-[3-(3-((3-fluoroanilinocarbonyl)hydrazono)-2-oxo-1-pentyl-2,3-dihydro-1H-indol-5-ylsulfanyl)propyl]benzoic acid;
61. 4-[3-(3-((3-fluoroanilinocarbonyl)hydrazono)-2-oxo-1-hexyl-2,3-dihydro-1H-indol-5-ylsulfanyl)propyl]benzoic acid;
62. 4-[3-(3-((anilinocarbonyl)hydrazono)-2-oxo-1-hexyl-2,3-dihydro-1H-indol-5-ylsulfanyl)propyl]benzoic acid;
63. 4-[3-(3-((3-fluoroanilinocarbonyl)hydrazono)-2-oxo-1-hexyl-2,3-dihydro-1H-indol-5-ylsulfanyl)ethyl]benzoic acid;
64. 4-(3-{1-hexyl-3-[(morpholine-4-carbonyl)hydrazono]-2-oxo-2,3-dihydro-1H-indol-5-ylsulfanyl}propyl)benzoic acid;
65. 4-[3-(3-((anilinocarbonyl)hydrazono)-2-oxo-1-pentyl-2,3-dihydro-1H-indol-5-ylsulfanyl)propyl]benzoic acid;
66. 4-(3-{3-[(morpholine-4-carbonyl)hydrazono]-2-oxo-1-propyl-2,3-dihydro-1H-indol-5-ylsulfanyl}propyl)benzoic acid;
67. 4-(3-{3-[(morpholine-4-carbonyl)hydrazono]-2-oxo-1-pentyl-2,3-dihydro-1H-indol-5-ylsulfanyl}propyl)benzoic acid;
68. 4-{2-[3-((4-fluoroanilinocarbonyl)hydrazono)-2-oxo-1-propyl-2,3-dihydro-1H-indol-5-ylsulfanyl]ethyl}benzoic acid;
69. 4-[3-(3-((3-fluoroanilinocarbonyl)hydrazono)-2-oxo-1-propyl-2,3-dihydro-1H-indol-5-ylsulfanyl)propyl]benzoic acid;
70. 4-{2-[3-((anilinocarbonyl)hydrazono)-2-oxo-1-hexyl-2,3-dihydro-1H-indol-5-ylsulfanyl]ethyl}benzoic acid;
71. 4-[3-(3-((4-fluoroanilinocarbonyl)hydrazono)-2-oxo-1-pentyl-2,3-dihydro-1H-indol-5-ylsulfanyl)propyl]benzoic acid;
72. 4-(2-{3-[(morpholine-4-carbonyl)hydrazono]-2-oxo-1-pentyl-2,3-dihydro-1H-indol-5-ylsulfanyl}ethyl)benzoic acid;
73. 4-(2-{1-hexyl-3-[(morpholine-4-carbonyl)hydrazono]-2-oxo-2,3-dihydro-1H-indol-5-ylsulfanyl}ethyl)benzoic acid;
74. 4-[3-(3-((4-fluoroanilinocarbonyl)hydrazono)-2-oxo-1-hexyl-2,3-dihydro-1H-indol-5-ylsulfanyl)propyl]benzoic acid;
75. 4-[3-(3-((3-trifluoromethylanilinocarbonyl)hydrazono)-2-oxo-1-pentyl-2,3-dihydro-1H-indol-5-ylsulfanyl)propyl]benzoic acid;
76. 4-[3-(3-((3-trifluoromethylanilinocarbonyl)hydrazono)-2-oxo-1-hexyl-2,3-dihydro-1H-indol-5-ylsulfanyl)propyl]benzoic acid;
77. 4-{2-[3-((3-fluoroanilinocarbonyl)hydrazono)-2-oxo-1-pentyl-2,3-dihydro-1H-indol-5-ylsulfanyl]ethyl}benzoic acid;
78. 4-{2-[3-((3,4-dichloroanilinocarbonyl)hydrazono)-2-oxo-1-propyl-2,3-dihydro-1H-indol-5-ylsulfanyl]ethyl}benzoic acid;
79. 4-{2-[3-((anilinocarbonyl)hydrazono)-2-oxo-1-propyl-2,3-dihydro-1H-indol-5-ylsulfanyl]ethyl}benzoic acid;
80. 4-{2-[3((1,3-thiazol-2-amine)carbonylhydrazono)-2-oxo-1-pentyl-2,3-dihydro-1H-indol-5-ylsulfanyl]ethyl}benzoic acid;
81. 4-{2-[3-((3-trifluoromethylanilino)carbonylhydrazono)-2-oxo-1-hexyl-2,3-dihydro-1H-indol-5-ylsulfanyl]ethyl}benzoic acid;
82. 4-{2-[3-((4-fluoroanilino)carbonylhydrazono)-2-oxo-1-hexyl-2,3-dihydro-1H-indol-5-ylsulfanyl]ethyl}benzoic acid;
83. 4-{2-[3-((2-chloroanilino)carbonylhydrazono)-2-oxo-1-heptyl-2,3-dihydro-1H-indol-5-ylsulfanyl]ethyl}benzoic acid;
84. ethyl 4-{2-[3-((2-chloroanilinocarbonyl)hydrazono)-2-oxo-1-heptyl-2,3-dihydro-1H-indol-5-ylsulfanyl]ethyl}benzoate;
85. 4-{2-[3((1H-imidazol-2-amine)carbonylhydrazono)-2-oxo-1-pentyl-2,3-dihydro-1H-indol-5-ylsulfanyl]ethyl}benzoic acid;
86. 4-{2-[3((thiophen-2-ylamine)carbonylhydrazono)-2-oxo-1-heptyl-2,3-dihydro-1H-indol-5-ylsulfanyl]ethyl}benzoic acid;
87. 4-{2-[3((1,3-thiazol-2-amine)carbonylhydrazono)-2-oxo-1-heptyl-2,3-dihydro-1H-indol-5-ylsulfanyl]ethyl}benzoic acid;
88. 4-{2-[3((1H-imidazol-2-amine)carbonylhydrazono)-2-oxo-1-heptyl-2,3-dihydro-1H-indol-5-ylsulfanyl]ethyl}benzoic acid;
89. 4-{3-[3((thiophen-2-ylamine)carbonylhydrazono)-2-oxo-1-pentyl-2,3-dihydro-1H-indol-5-ylsulfanyl]propyl}benzoic acid;
90. 4-{3-[3((1,3-thiazol-2-amine)carbonylhydrazono)-2-oxo-1-pentyl-2,3-dihydro-1H-indol-5-ylsulfanyl]propyl}benzoic acid;
91. 4-{3-[3((1H-imidazol-2-amine)carbonylhydrazono)-2-oxo-1-pentyl-2,3-dihydro-1H-indol-5-ylsulfanyl]propyl}benzoic acid;
92. 4-{3-[3((thiophen-2-ylamine)carbonylhydrazono)-2-oxo-1-heptyl-2,3-dihydro-1H-indol-5-ylsulfanyl]propyl}benzoic acid;
93. 4-{3-[3((1,3-thiazol-2-amine)carbonylhydrazono)-2-oxo-1-heptyl-2,3-dihydro-1H-indol-5-ylsulfanyl]propyl}benzoic acid;
94. 4-{3-[3((1H-imidazol-2-amine)carbonylhydrazono)-2-oxo-1-heptyl-2,3-dihydro-1H-indol-5-ylsulfanyl]propyl}benzoic acid; and mixtures thereof.

15. The biaromatic compound as defined by claim 1, wherein formula (I) at least one of the following conditions is satisfied:
R1 is a methyl, propyl, butyl, pentyl, hexyl or heptyl radical, a benzyl radical or a phenethyl radical;
R2 is a hydrogen atom, an ethyl or tert-butyl radical, an unsubstituted phenyl radical, a phenyl radical mono- or disubstituted with a fluorine or chlorine atom or a CF₃ radical, a benzyl radical, a phenylsulfonyl radical substituted with a methyl radical, or an unsubstituted thiophenyl, thiazolyl or imidazolyl radical;
R′2 is a hydrogen atom;
with the proviso that R2 and R′2 can together form a morpholino radical,
R3 is a hydrogen atom or a methyl or ethyl radical.

16. A cosmetic composition comprising a cosmetically effective amount of at least one biaromatic compound as defined by claim 1, formulated into a cosmetically and physiologically acceptable support therefor.

17. The cosmetic composition as defined by claim 16, comprising from 0.001% to 3% by weight of said at least one biaromatic compound.

18. The cosmetic composition as defined by claim 16, formulated for body or hair hygiene.

19. A pharmaceutical composition comprising a pharmaceutically effective amount of at least one biaromatic compound as defined by claim 1, formulated into a pharmaceutically and physiologically acceptable support therefor.

20. The pharmaceutical composition as defined by claim 19, comprising from 0.001% to 10% by weight of said at least one biaromatic compound.

21. A regime or regimen for regulating and/or restoring the metabolism of skin lipids, comprising administering to an individual in need of such treatment, a thus effective amount of the pharmaceutical composition as defined by claim 19.

22. A regime or regimen:
1) for treating dermatological conditions or afflictions linked to a disorder of keratinization involving differentiation and proliferation, for treating acne vulgaris, comedonic or polymorphic acne, acne rosacea, nodulocystic acne, acne conglobata, senile acne or secondary acnes, solar, drug or occupational acne,
2) for treating other types of disorders of keratinization, ichthyoses, ichthyosiform conditions, Darier's disease, palmoplantar keratoderma, leucoplakia and leucoplakiform conditions, or cutaneous or mucosal (oral) lichen,
3) for treating other dermatological conditions, disorders or afflictions having an inflammatory immunoallergic component, with or without cell proliferation disorder, and all forms of psoriasis, whether cutaneous, mucosal or ungual, and psoriatic rheumatism, or, alternatively, cutaneous atopy, eczema, or respiratory atopy or, alternatively, gingival hypertrophy,
4) for treating all dermal or epidermal proliferations, whether benign or malignant and whether or not of viral origin, common warts, flat warts and epidermodysplasia verruciformis, florid or oral papillomatoses, T lymphoma, and the proliferations which can be induced by ultraviolet radiation, basal cell and prickle cell epithelioma, and also all precancerous skin lesions, keratoacanthomas,
5) for treating other dermatological disorders, conditions or afflictions, immune dermatoses, lupus erythematosus, immune bullous diseases and collagen diseases, scleroderma,
6) for the treatment of dermatological or general conditions having an immunological component,
7) for the treatment of skin disorders due to exposure to UV radiation, and for repairing or combating skin aging, whether photoinduced or chronologic, or for reducing actinic keratoses and pigmentations, or any pathology associated with chronologic or actinic aging, xerosis,
8) for combating disorders of the sebaceous function, hyperseborrhoea of acne or simple seborrhoea,
9) for preventing or treating disorders of cicatrization or for preventing or repairing stretch marks,
10) for the treatment of disorders of pigmentation, hyperpigmentation, melasma, hypopigmentation or vitiligo,
11) for the treatment of conditions of the metabolism of lipids, obesity, hyperlipidaemia or non-insulin-dependent diabetes,
12) for the treatment of inflammatory conditions, arthritis,
13) for the treatment or prevention of cancerous or precancerous conditions,
14) for the prevention or treatment of alopecia of various origins, alopecia due to chemotherapy or to radiation,
15) for the treatment of disorders of the immune system, asthma, type I diabetes mellitus, multiple sclerosis or other selective dysfunctions of the immune system,
16) for the treatment of conditions of the cardiovascular system, arteriosclerosis or hypertension, comprising administering to an individual in need of such treatment, a thus effective amount of the pharmaceutical composition as defined by claim 19.
